(19) United States
(12) Patent Application Publication
(10) Pub. No.: US 2011/0136123 A1
(43) Pub. Date: Jun. 9, 2011

(54) ALTERNATIVE SPLICING GENE VARIANTS IN CANCER

(75) Inventors: Roscoe Klinck, Sherbrooke (CA); Sherif Abou Elela, Stoke (CA); Benoit Chabot, Sherbrooke (CA)

(73) Assignee: UNIVERSITE DE SHERBROOKE, Sherbrooke, QC (CA)

(21) Appl. No.: 12/673,265

(22) PCT Filed: Aug. 15, 2008
(86) PCT No.: PCT/CA08/01477

§ 371 Date: Feb. 12, 2010

Related U.S. Application Data
(60) Provisional application No. 60/935,489, filed on Aug. 15, 2007.
(60) Provisional application No. 60/988,213, filed on Nov. 15, 2007.

(51) Int. Cl. C12Q 1/68

(52) U.S. Cl. 435/6.12; 435/6.14

(57) ABSTRACT

The present invention relates to a method to identify alternatively spliced variants enriched in cancer specimens and a method for prognosis of cancer in a subject by detecting a signature of splicing events. There is also provided a method for profiling cancer in a subject by detecting a signature of splicing events.

### TECHNICAL FIELD

The present invention relates to a method to identify alternatively spliced variants enriched in cancer specimens.

### BACKGROUND OF THE INVENTION

The transformation of a normal cell into a malignant cell results, among other things, in the uncontrolled proliferation of the progeny cells, which exhibit immature, undifferentiated morphology, exaggerated survival and proangiogenic properties and expression, overexpression or constitutive activation of oncogenes not normally expressed in this form by normal, mature cells.

Nearly all cancers are caused by abnormalities in the genetic material of the transformed cells. These abnormalities may be due to the effects of carcinogens, such as tobacco smoke, radiation, chemicals, or infectious agents. Other cancer-promoting genetic abnormalities may be randomly acquired through errors in DNA replication, or are inherited, and thus present in all cells from birth. Complex interactions between carcinogens and the host genome may explain why only some develop cancer after exposure to a known carcinogen. New aspects of the genetics of cancer pathogenesis, such as DNA methylation, and microRNAs are increasingly being recognized as important.

One example of cancer is epithelial ovarian cancer which is the second most common gynecological cancer and the deadliest amongst gynecological pelvic malignancies. Early symptoms of ovarian cancer are often mild and unspecific, making this disease difficult to detect. In most cases, at the time of diagnosis, cancer cells have already disseminated throughout the peritoneal cavity. In fact, over 70% of patients are diagnosed with late stage disease and only a minority survive over 5 years post-diagnosis. Early detection offers a 90% 5-year survival rate. The inability to detect ovarian cancer at an early stage and its propensity for peritoneal metastasis are largely responsible for these low survival rates.

Currently, there are no reliable methods for detecting early stages of epithelial ovarian cancer. Blood level of CA125 tumour antigen is employed as a predictor of clinical recurrence of ovarian cancers, and to monitor response to anticancer therapy (Yang et al., 1994, Zhonghua Fu Chan Ke Za Zhi, 29: 147-149). The CA125 serum marker combined with transvaginal ultrasonography are the current clinical tests offered for screening for early stages of ovarian cancer in high risk populations (Nikolic et al., 2006, Bosn J Basic Med Sci 6: 3-6). However, neither of these modalities individually or combined have proven reliable (Nikolic et al., 2006, Bosn J Basic Med Sci 6: 3-6), and there is an urgent need to develop new screening tests to detect epithelial ovarian cancer at an early stage.

Epithelial ovarian tumours are heterogeneous and include many different histopathological subtypes: serous, endometrioid, mucinous, clear cell, undifferentiated or mixed. The serous type is the most frequent and the second most lethal. Recent studies have focused on differences in molecular profiling of gene expression patterns to uncover diagnostic and prognostic markers as well as new therapeutic targets in a variety of cancers. Although promising results have been reported in some cancers, the genes that are differentially expressed between normal and cancer cells seem to vary between individual microarray studies, reflecting either a variability in methods and in the choice of model systems or a heterogeneity in selected tissues (Kopper & Timar, 2005, Pathol Oncol Res, 11: 197-203).

Another example of cancer is breast cancer. Breast cancer is the fifth most common cause of cancer death (after lung cancer, stomach cancer, liver cancer and colon cancer). Among women worldwide, breast cancer is the most common cause of cancer death. There are numerous ways breast cancer is classified. Like most cancers, breast cancer can be divided into groups based on the tissue of origin, e.g. epithelial (carcinoma) versus stromal (sarcoma). The vast majority of breast cancers arise from epithelial tissue, i.e. they are carcinomas.

Breast cancer is diagnosed by the examination of surgically removed breast tissue. A number of procedures can obtain tissue or cells prior to definitive treatment for histological or cytological examination. Such procedures include fine-needle aspiration, nipple aspirates, ductal lavage, core needle biopsy, and local surgical excision. These diagnostic steps, when coupled with radiographic imaging, are usually accurate in diagnosing a breast lesion as cancer. Occasionally, pre-surgical procedures such as fine needle aspirate may not yield enough tissue to make a diagnosis, or may miss the cancer entirely. Imaging tests are sometimes used to detect metastasis and include chest X-ray, bone scan, Cat scan, MRI, and PET scanning. While imaging studies are useful in determining the presence of metastatic disease, they are not in and of themselves diagnostic of cancer. Only microscopic evaluation of a biopsy specimen can yield a cancer diagnosis. Ca 15.3 (carbohydrate antigen 15.3, epithelial mucin) is a tumor marker determined in blood which can be used to follow disease activity over time after definitive treatment. Blood tumor marker testing is not routinely performed for the screening of breast cancer, and has poor performance characteristics for this purpose.

Thus, detection of many cancers still relies on detection of an abnormal mass in the organ of interest. In many cases, a tumor is often detected only after a malignancy is advanced and may have metastasized to other organs. For example, breast cancer is typically detected by obtaining a biopsy from a lump detected by a mammogram or by physical examination of the breast. Also, although measurement of prostate-specific antigen (PSA) has significantly improved the detection of prostate cancer, confirmation of prostate cancer typically requires detection of an abnormal morphology or texture of the prostate. Thus, there is a need for methods for earlier detection of cancer. Such new methods could, for example, replace or complement the existing ones, reducing the margins of uncertainty and expanding the basis for medical decision making.

It would be highly desirable to be provided with novel biomarkers for the early detection, prognosis and clinical management of cancers. Sensitive and specific tests that can diagnose different stages of cancer would greatly improve patient survival rates by facilitating early diagnosis and tailored therapies. It would also be highly desirable to be provided with new screening tests to detect cancer at an early stage.

### SUMMARY OF THE INVENTION

The present invention relates to a method to identify alternatively spliced variants enriched in cancer specimens.

In another embodiment, the cancer that can be detected in these cancer specimens is selected from the group consisting of breast cancer, glioma, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma, testicular cancer, oral cancer, pharyngeal cancer, pediatric neoplasms, leukemia, neuroblastoma, retinoblastoma, glioma, rhabdomyoblastoma and sarcoma.

In one aspect, a method for prognosis of cancer in a subject by detecting a signature of splicing events comprising the steps of obtaining a nucleic acid sample from said subject, and determining whether the nucleic acid sample contains a signature specific to a cancer is disclosed.

There is also provided a method for profiling cancer in a subject by detecting a signature of splicing events comprising the steps of obtaining a nucleic acid sample from said subject, and determining whether the nucleic acid sample contains a signature specific to a cancer.

In a preferred embodiment, the signature comprises at least 1 splicing variant.

Further, the method disclosed herein also can comprise an initial step of designing at least two independent PCR primer pairs for each predicted exon-exon junction from a transcript map of a gene affected in a cancer.

In addition, the method disclosed herein can comprise a step of PCR amplifying the nucleic acid sample with the PCR primer pairs to obtain amplicons.

Further, the method disclosed herein can comprise the step of measuring the size and sequence of said amplicons.

Also in accordance with the present invention, there is disclosed a method for identifying a signature specific of a cancer, said signature consisting of at least one specific splicing event or a specific combination of splicing events, said method comprising the steps of designing at least two independent PCR primer pairs for each predicted exon-exon junction from a transcript map of a gene affected in cancer; reverse transcribing a template from RNA from a sample of cancer tissue and a sample from normal tissue; amplifying amplicons of said gene by PCR with the PCR primers pairs using the template reverse transcribed from the cancer tissue and the normal tissue; and determining the size and sequence of said amplicons; wherein the presence of said at least one alternative splicing event corresponds to the signature of the cancer.

Further, the method disclosed herein can comprise the step of performing a comparative analysis of amplicons obtained from the template reverse transcribed from the cancer tissue and the normal tissue.

Furthermore, the method disclosed herein can comprise the step of identifying the presence of at least one alternative splicing event in the gene.

In a preferred embodiment, the PCR primer pairs are designed to amplify amplicons ranging from 100 to 700 base pairs.

In another embodiment, the step of amplifying is carried out in a liquid handling system linked to a thermocycler.

Furthermore, the method disclosed herein can comprise the step of selecting amplicons with a difference of at least 10% of points between a mean Ψs for normal and cancer tissue and with a maximum standard deviation of the Ψs for each tissue type of at most 26%.

In accordance with the present invention, there is also provided a diagnostic kit for detecting a signature of ovarian cancer in a patient comprising PCR primer pairs for predicted exon-exon junctions of at least one splicing variant; and a set of instructions for using said primers to generate and detect a signature specific of a cancer, said signature consisting of at least one splicing variant or a specific combination of splicing variants.

In addition, the kit disclosed herein can also comprise a transcript map.

In another embodiment, the splicing variants occur in genes selected from the group consisting of AFF3, AGR3, APP, AXIN1, BMP4, BTC, C11orf17, CADM1, CCNE1, CHEK2, DNMT3B, FANCA, FANCL, FGFR1, FGFR2, FGFR4, FN1-EDA, FIN-EDB, FIN-IIICS, GATA3, GNB3, GPR137, HMGA1, HSC, IGSF4, KITLG, LGALS9, MCL1, NRG1, NUP98, PAXIP1, PLD1, POLI, POLM, PSAP, PTK2, PTPN13, RAD52, SHMT1, SLIT2, SRP19, STIM1, SYK, SYNE2 TOPBP1, TSSC4, TUBA4A and UTRN.

In another embodiment, the splicing variants occur in genes selected from the group consisting of ADAM15, BCAS1, C11ORF4, CCL4, CTNNA1, DDR1, DRF1, DSC3, ECGF1, ECT2, FN1, F3, H63, HMGA1, HMMR, INSR, LIG3, LIG4, NOTCH3, PACE4, POLB, PTPRB, RSN, RUNX2, SHC1, TLK1 and TNFRSF5.

In a preferred embodiment, the splicing events are selected from the group consisting of an alternative 3′ splicing, an alternative 5′ splicing, an alternative 3′ and 5′ splicing, a cassette exon and alternative 5′ or 3′ splicing, a multiple cassette exon splicing, a mutually exclusive exon splicing and a cassette exon splicing. Further, said splicing events are alternative cassette exon events.

In another embodiment, the splicing variant is:

(SEQ ID NO: 3061)
ggttcaccca ccagagtgat ntgtggagtt atggtgtgac tgtgtgggag ctgatgactt
ttggggccaa accttacgat gggatcccag cccgggagat ccctgacctg ctggaaaagg
gggagnnnnt gccccagccc cccatatgca ccattgatgt ctacatgatc atggtcaaat
gtgcgtggct gagctgtgct ggctgcctgg aggagggtgg gaggtcct.

### BRIEF DESCRIPTION OF THE DRAWINGS

Having thus generally described the nature of the invention, reference will now be made to the accompanying drawings, showing by way of illustration, preferred embodiments thereof, and in which:

FIG. 1 illustrates a layered and integrated system for splicing isoform annotation (LISA), wherein in (A) it is shown an overview of the LISA annotation process; in (B) a transcript map generated for the stromal interaction molecule 1 (STIM1) is shown as an example, wherein each variant transcript from AceView is shown on a separate line and named (left) as per the AceView convention, exons are shown as pale boxes, the intervening introns are shown as lines (not to scale), the relative locations of forward (green) and reverse (red) primers are shown as vertical lines, the designed PCR reactions are shown below each targeted transcript (black horizontal lines), and other relevant information, such as coding regions (dashed horizontal lines) or protein functional domains (magenta horizontal lines) are mapped onto this representation and can be accessed for subsequent data analysis; in (C) capillary electropherograms of the PCR reaction spanning AS event 1 of the STIM1 gene shown in (B) in a normal ovary and an epithelial ovarian cancer (EOC) pool wherein each reaction is compared to internal markers at 15 (M₁₅) and 7000 (M₇₀₀₀) base pairs and wherein amplicon sizes and concentrations relative to the markers are measured and digitized, and wherein the fluorescence signals in arbitrary units (au) of the short and long isoforms are indicated; in (D) graphical representation of the results obtained from the PCR reactions targeting the STIM1 AS event 1 in 4 RNA sources wherein each row represents data from a single RNA source, and each column represents a single PCR reaction spanning an AS event and wherein electropherograms were analyzed for the presence of expected amplicon sizes and the most intense amplicon signal for each reaction in all sources was identified and wherein the ratio of this amplicon relative to the total expected amplicon concentration was calculated and expressed as the percentage splicing index, Ψ;

FIG. 2 illustrates an example of splicing annotations generated by LISA, such as in (A) it is shown a LISA generated displays of the cancer-associated gene SHMT1 wherein exon sizes (black rectangles) are proportional to the square root of their lengths, introns (white rectangles) are not to scale, all putative exon-exon junctions are automatically assigned (uppercase letters in the intervening introns), and wherein the alternative splicing (AS) events are identified, classified by type and listed beneath the transcript representation (labeled by roman numerals); in (B) a LISA-generated summary of RNA source specific detection of SHMT1 exon-exon junctions is shown, wherein the exon-exon junction analysis was performed for each of 2 ovarian cancer tissue pools and 2 normal tissue pools, whereas columns represent the data for each exon-exon junction defined in (A), and each row represents a different RNA source, junctions are classified as detected (green), not detected (red) or, when data is ambiguous, not determined (white), and data from all RNA sources were subjected to unsupervised clustering and displayed with a dendrogram (left) representing the similarities between the RNA sources used; in (C) RNA source-based display of AS events is exemplified, wherein the data generated in (B) was further analyzed to assess the state of each detected AS event, AS events defined in (A), (columns), are classified as yielding a long form, a short form or both for each RNA source (rows), such as the presence (green) or absence (red) of the long form is indicated in the left semicircle for each event in each RNA source, likewise for the short form's state is indicated in the right semicircle, and wherein the data from each RNA source is clustered and presented with its corresponding dendrogram (left) to emphasize RNA source similarities;

FIG. 3 illustrates four-phase pipeline for the identification of serous ovarian cancer associated alternative splicing (AS) events, wherein the discovery screen was conducted using 2 pools of RNA extracted from 4 different normal tissues and 2 pools of RNA extracted from 4 different epithelial ovarian cancer (EOC) tissues, a total of 600 ovarian associated genes were analyzed using a comprehensive set of PCR primers that span all possible splicing events, and the AS events showing different profiles in normal and tumour tissue pools were selected for the validation screen, and for this screen, 104 putative cancer-associated AS events, derived from 98 genes were analyzed in 25 normal and 21 tumour samples;

FIG. 4 illustrates a distribution of the different types of alternative splicing (AS) events in ovarian cancer associated genes, wherein in (A) the distribution of the predicted and LISA validated AS events in 182 ovarian cancer-specific genes is shown, wherein AS events are categorized into seven types: alternative 3′ or 5′ or both (Alt. 3′, Alt. 5′, Alt. 3′ & 5′), single or multiple cassette exon (Cass., Mult. cass.), cassette exon and alternative 3′ or 5′ (Cass. & alt 3′ or 5′), and mutually exclusive exons (Mut. Excl.), and wherein bar height represents total number of AceView predicted AS events of each type, and these events were either validated (black) in at least one pool sample, or not validated (grey) in any of the four ovarian tissue pools; in (B) a comparison of the distribution of the detected AS events following the discovery screen, and cancer-associated AS events identified following the validation screen is shown;

FIG. 5 illustrates novel splicing event in ERBB2 mRNA identified by the LISA, wherein two AceView rendered transcripts of ERBB2 are shown schematically (top), exon sizes (black rectangles) are proportional to the square root of their lengths, introns (white rectangles) are not to scale, and further wherein in the detailed representation of the region of interest (bottom), the exons are shown as blue rectangles with numbers indicating their sizes in nucleotides (nt), relative positions of forward (green) and reverse (red) primers are shown, and marker peaks at 15 bp (M15) and 7000 bp (M7000) are also shown;

FIG. 6 illustrates a summary of the serous ovarian cancer associated alternative splicing (AS) events, wherein the clustering of ovarian cancer associated AS variation of 45 genes (columns) analyzed in 50 ovarian tissue samples (rows) is shown, the splicing variations are presented in scaled percent splicing index (ψ) values shown in a gradation of colors representing the number of standard deviations from the mean value (Z score), the tissue hierarchical clustering is shown as a dendrogram (left), the column on left shows clustered positions of normal (green) and serous tumour tissues (red), wherein the columns are ordered according to the difference of the mean ψ values of normal and tumour samples. ψ values are high (tendency towards blue color) in normal tissue for the 24 genes on the left, and high in tumour tissue for the remaining 21 genes;

FIG. 7 illustrates a quantitative PCR for a subset of 11 validated genes in 2 ovarian normal and 2 serous tumour pools, wherein Relative quantitation (RQ), calculated using the ΔΔCt method, relative to Normal Pool 1 which was normalized to a value of 1 is shown.

### DETAILED DESCRIPTION OF THE PREFERRED EMBODIMENT

In accordance with the present invention, there is provided a method to identify alternatively spliced variants enriched in cancer specimens.

The term “cancer” includes but is not limited to, breast cancer, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer (generally considered the same entity as colorectal and large intestinal cancer), fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma (also known as intraocular melanoma), testicular cancer, oral cancer, pharyngeal cancer or a combination thereof. In an embodiment, the cancer is a brain tumor, e.g. glioma. In another embodiment, the cancer expresses the HER-2 or the HER-3 oncoprotein. The term “cancer” also includes pediatric cancers, including pediatric neoplasms, including leukemia, neuroblastoma, retinoblastoma, glioma, rhabdomyoblastoma, sarcoma and other malignancies.

Accordingly, there is provided a method of identifying new markers characteristic of a signature for a cancer.

Alternative splicing of pre-mRNA is a post-transcriptional process that allows the production of distinct mRNAs from a single gene with the potential to expand protein structure and diversity. Alternative splicing can also introduce or remove regulatory elements to affect mRNA translation, localization or stability. More than 70% of human genes may undergo alternative splicing with many genes capable of producing dozens and even hundreds of different isoforms.

In multicellular organisms, alternative splicing is a process that is tightly regulated during development and in different tissues. Inherited and acquired changes in pre-mRNA splicing patterns have been associated with several human diseases including cancer (Venables, 2006, Bioessays, 28: 378-386). Some of these changes can arise from mutations at either the splice sites or within proximal splicing enhancer or silencer elements (Pagani & Baralle, 2004, Nat Rev Genet, 5: 389-396). In other cases, variations in the expression of trans-acting splicing factors have been observed (Brinkman, 2004, Clin Biochem, 37: 584-594). A direct effect in splice site use resulting in the production of cancer-specific splice isoforms has been observed in a few cases (Karni et al., 2007, Struct Mol Biol, 14: 185-193). Cancer-specific alterations in splice site selection can affect genes controlling cellular proliferation (e.g., FGFR2, p53, MDM2, FHIT and BRCA1), cellular invasion (e.g., CD44, Ron), angiogenesis (e.g, VEGF), apoptosis (e.g, Fas, Bcl-x and caspase-2) and multidrug resistance (e.g., MRP-1).

Following initial computational efforts designed to exploit collections of expressed sequence tag (EST) databases, there has been an increase in high-throughput experimental approaches to identify changes in splicing events under a variety of conditions. Oligonucleotide-based microarray technologies have been introduced to identify global alternative splicing events and to examine changes in the alternative splicing of a large collection of events. These approaches are useful for monitoring the expression of known splice variants. However, they are not designed to discover novel splice sites, nor do they provide information on the combinatorial patterns of exon inclusion/skipping in the same gene. Furthermore, the lack of standardized analysis and normalization can compromise the interpretation of the results.

Arrays made from alternative splice junction probes have been used to detect splicing changes in Hodgkin Lymphoma (Relogio et al., 2005, J Biol Chem, 280: 4779-4784) and breast cancer cell lines and xenografts (Li et al., 2006, Cancer Res, 66: 1990-1999). A related medium-throughput technique has been used to show that alternative splicing analysis can complement the power of gene expression analysis of prostate tumours (Li et al., 2006, Cancer Res, 66: 4079-4088; Zhang et al., 2006, BMC Bioinformatics, 7: 202). So far, 30 different genes have been shown to be alternatively spliced in a cancer-specific manner (Venables, 2004, Cancer Res, 64: 7647-7654). In ovarian tumours specifically, three genes have been reported to be regulated at the level of splicing (He et al., 2007, Oncogene, advance online publication, Feb. 19, 2007; Sigalas et al., 1996, Nat Med 2: 912-917).

Thus, in the present invention, there is disclosed a layered and integrated system for splicing isoform annotation platform (LISA). LISA relies on automated RT-PCR technology that generates tissue-specific annotation of alternative splicing events. The bioinformatics infrastructure supporting the annotation effort helps assess the potential functional impact of individual alternative splicing events and allows adaptable visualization of large sets of validated results.

The LISA is used in a preferred embodiment to identify alternatively spliced variants enriched in cancer specimens. There is reported herein a set of highly significant and biologically relevant splicing differences that make up a strong signature for cancer samples.

The signature of a cancer sample consists in the presence of alternatively spliced variants in the sample, More specifically, the signature is composed of at least one gene, which discriminates between a cancerous tissue and normal tissue with about 90% accuracy. More preferably, the signature is composed of at least 2, 3, 4 or 5 variants, or markers, disclosed for example in Table 1 herein below, more preferably at least 6, 7, 8, 9, or 10, preferably 15, 20, 25, 30, 35, 40, 45, more preferably 48 variants. Thus, the combination of more than one alternative spliced variant can also be used as a signature.

In order to identify such alternatively spliced variants, there is disclosed a method using the layered and integrated system described herein. As a first step, a map of splicing events is generated. A list of genes potentially involved in cancer such as, for example in ovarian or breast cancer, is first obtained by screening databases. Then, the exon structure of each gene is determined. All splice sites are identified, generating the splicing map. The following step consists in designing PCR primers and designing PCR reactions to cover all putative exon-exon junctions identified on the splicing map. Following, the RNA isolated from samples from “normal tissues” (without cancer) and from samples positive for a specific cancer is reverse transcribed in bulk. The DNA obtained from the reverse transcription is then used as template for the PCR reactions conceived previously, also using the PCR primers designed previously. Once PCR amplicons are obtained, the amplicons from normal tissues are compared to those obtained from cancer tissues. Splicing events are thus identified following this comparison. The method described herein allows identification of splicing events which will be part of a cancer signature and will thus allow prognosing or profiling the presence of the target cancer in a patient by identifying the presence of this signature, i.e. the presence of one or more alternative splicing events occurring in cancer samples and not occurring in normal samples.

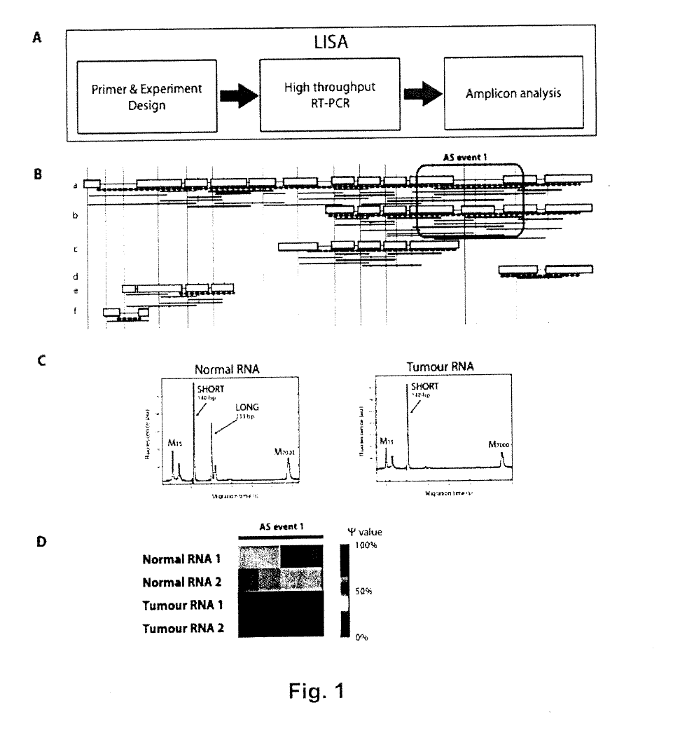

The LISA uses RT-PCR to provide a systematic and comprehensive coverage of alternative splicing events. As illustrated in FIG. 1A, LISA includes a computational automated framework for high throughput RT-PCR analysis of splicing isoforms. Within the system, a transcript map containing publicly available mRNAs and ESTs for each gene is generated and sets of PCR primers and experiments are designed such that all putative exon-exon junctions and alternative splicing events are covered by at least two distinct PCR reactions. The data are transferred to the LISA database and analyzed to identify amplicons. Transcript information for each selected gene is uploaded into the LISA database from AceView. The system automatically designs PCR primers and PCR reactions to cover all putative exon-exon junctions. RNA is reverse transcribed in bulk, using a mixture of random hexamers and poly (T) oligonucleotides. The experiment design is sent to an automated platform that performs the PCR reactions in 384 well plates and separates and quantifies the resulting amplicons by capillary electrophoresis. Digitized experimental data is merged with the transcript input for analysis. PCR reactions are carried out, for example, using a liquid handling system linked to thermocyclers, and the amplified products are analyzed by, for example and not restricted to, an automated chip-based capillary electrophoresis.

Contrary to current approaches that identify tissue-specific variations in alternative splicing profiles relying heavily on microarray analysis to produce large quantities of data that must further be validated by RT-PCR, a preferred embodiment is directed to a method that directly inspects hundreds of genes by RT-PCR without recourse to cumbersome slab gel methods. LISA effectively fills a gap between large-scale microarray studies and individual gene investigations, providing an alternative to array-based expression profiling.

As disclosed herein below, the LISA was used to provide high quality comprehensive annotation of alternative splicing for 600 genes in 46 different tissues. The analysis required nearly 100 000 RT-PCR reactions that were carried out in less than eight weeks.

One major advantage of LISA is the associated in silico filtering modules that can combine alternative splicing data with queries on sequence or coding information, such as Pfam domains, putative RNA secondary structure, and single nucleotide polymorphisms.

Furthermore, the encompassed method herein further comprises an initial step of verifying the tissue-specific representation of expression data in such databases. Depending on the result of this assessment, the coverage of each gene could be modified accordingly. Poorly represented tissues would benefit from a complete annotation strategy, as employed here, whereas for well represented tissues, the design module could be modified to focus only on EST supported alternative splicing events. This would allow gene analysis to be performed with limited number of PCR reactions, enabling the screening of many more genes or tissue specimens with the same total number of reactions.

In one example of a screen presented here, with only 600 genes, 48 splicing events not previously detected in ovarian cancers were identified.

The majority (>80%) of the identified cancer-specific alternative splicing events are exon cassettes that extended the coding portions of genes. (see Table 1). For example, the short DNMT3B isoform is lacking part of the catalytic DNA methyltransferase domain, including the TRD loop previously shown to be important for cytosine recognition, and is therefore inactive. Another example where alternative splicing affects function concerns the growth factor KITLG. In this case, the skipped exon encodes a metalloprotease cleavage site that determines whether KITLG will be membrane-bound or secreted. The transmembrane form is more active in promoting cell-cell adhesion, cell proliferation and survival by inducing more persistent tyrosine kinase activation than the secreted isoform. The overall preferential enrichment of in-frame alternative cassette exons within functional domains of ovarian cancer-associated genes suggests that alternative splicing of these genes contributes to ovarian tumour biology.

TABLE 1
Properties of ovarian cancer-specific alternative splicing (AS) variants.

| | | | | | Epithelial ovarian |
|---|---|---|---|---|---|
| | | | | | cancer specific |
| Gene | | ASE¹ size, | in frame/in | | (CS) or epithelial |
| symbol | Gene name | type in EOC² | coding region | Function | specific (ES) |
| AFF3 | AF4/FMR2 family, | +75 nt; exon | Coding region in | Transcription | ES |
| | member 3 | | frame | factor | |
| AGR3 | Anterior gradient | −136 nt; exon | Removes ATG | Breast cancer | n.d. |
| (BCMP11) | homolog 3 | | Downstream | | |
| | | | initiation | | |
| APP | Amyloid beta (A4) | −57 nt; exon | Coding region | Transmembrane/ | CS |
| | precursor protein | | in frame | secreted | |
| AXIN1 | Axin 1 | −108 nt; exon | Coding region in | G protein | ES |
| | | | frame | signalling | |
| BMP4 | Bone morphogenetic | +209 nt; alt 5′ | 5′UTR | Bone growth | CS |
| | protein 4 | | | factor | |
| BTC | Betacellulin | −147 nt; exon | Coding region in | EGF family of | CS |
| | | | frame | growth factors. | |
| C11orf17 | C11orf17 | +81 nt; exon | Coding region in | PKA- | ES |
| | | | frame | interacting | |
| | | | | protein | |
| CADM1 | Cell adhesion molecule 1 | +33 nt; exon | Coding region in | Adhesion | CS |
| (IGSF4) | | | frame | | |
| CCNE1 | Cyclin E1 | +135 nt; exon | Coding region in | S phase | ES |
| | | | frame | progression | |
| CHEK2 | Checkpoint kinase 2 | +62 nt; exon | Out of frame | cell cycle | ES |
| | | | truncating | checkpoint | |
| | | | | regulator | |
| DNMT3B | DNA (cytosine-5-)- | +189 nt; | Coding region in | De novo | CS |
| | methyltransferase 3 beta | (2 exons) | frame | methylation | |
| FANCA | Fanconi anemia, | −129 nt; exon | Coding region in | Genome | ES |
| | complementation group A | | frame | stability | |
| FANCL | Fanconi anemia, | +278 nt; (4 | Out of frame | Stem cell | ES |
| | complementation group L | exons) | truncating | maintenance | |
| | | | Or alternate | | |
| | | | ATG used | | |
| FGFR1 | Fibroblast growth factor | +267 nt; exon | Coding region in | Mitogenesis | CS |
| | receptor 1 | | frame | and | |
| | | | | differentiation | |
| FGFR2 | Fibroblast growth factor | +267 nt; exon | Coding region in | Mitogenesis | ES |
| | receptor 2 | | frame | and | |
| | | | | differentiation | |
| FGFR4 | Fibroblast growth factor | +194 nt; alt 3′ | Longer form is | Mitogenesis | ES |
| | receptor 4 | | truncated | and | |
| | | | | differentiation | |
| FN1-EDA | Fibronectin | −270 nt; exon | Coding region in | Cell adhesion | ES |
| | | | frame | and migration | |
| FN1-EDB | | −273 nt; exon | Coding region in | | ES |
| | | | frame | | |
| FN1-IIICS | | −93 nt; intron | Coding region in | | ES |
| intron | | | frame | | |
| FN1-IIICS | | −75 alt 3′ | Coding region in | | ES |
| upstream | | | frame | | |
| GATA3 | GATA binding protein 3 | +143 nt; exon | Out of frame | Transcription | ES |
| | | | | factor | |
| GNB3 | Guanine nucleotide | +241 nt; intron | Removes ATG | Hypertension | CS |
| | binding protein (G | | Downstream | | |
| | protein), beta | | initiation | | |
| | polypeptide 3 | | | | |
| GPR137 | G protein-coupled | −376 nt; (2 | Out of frame | Unknown | ES |
| (C11orf4) | receptor 137 | exons) | truncating | | |
| HMGA1 | High mobility group AT- | +33 nt; alt 5′ | Coding region in | Transcription | ES |
| | hook 1 | | frame | factor | |
| HSCB | HscB iron-sulfur cluster | −145 nt; exon | Coding region in | Protein folding | CS |
| (HSC20) | co-chaperone homolog | | frame | chaperone | |
| KITLG | KIT ligand | −84 nt; exon | Coding region in | Tyrosine- | CS |
| | | | frame | kinase | |
| | | | | receptor ligand | |
| | | | | Cell migration | |
| LGALS9 | Lectin, galactoside- | +96 nt; exon | Coding region in | Modulating | CS |
| | binding, soluble, 9 | | frame | cell-cell and | |
| | (galectin 9) | | | cell-matrix | |
| | | | | interactions | |
| MCL1 | Myeloid cell leukemia | +248 nt; exon | Out of frame | Apoptosis | ES |
| | sequence 1 (BCL2- | | | | |
| | related) | | | | |
| NRG1 | Neuregulin 1 | −24 nt; exon | Coding region in | Glycoprotein | CS |
| | | | frame | Ligand of | |
| | | | | ERBB family | |
| NUP98 | Nucleoporin 98 kDa | −222 nt; exon | Coding region in | Signal- | CS |
| | | | frame | mediated | |
| | | | | nuclear | |
| | | | | transport | |
| PAXIP1 | PAX interacting (with | −71 nt; alt 5′ | Out of frame | Genome | ES |
| | transcription-activation | | truncating | stability, | |
| | domain) protein 1 | | | condensation | |
| | | | | of chromatin | |
| | | | | and mitotic | |
| | | | | progression | |
| PLD1 | Phospholipase D1, | −114 nt; exon | Coding region in | Regulation of | ES |
| | phosphatidylcholine- | | frame | mitosis | |
| | specific | | | | |
| POLI | Polymerase (DNA | +106 nt; intron | Out of frame | DNA damage | CS |
| | directed) iota | | truncating | checkpoint | |
| POLM | Polymerase (DNA | +270 nt; (2 | Coding region in | DNA repair | ES |
| | directed), mu | exons) | frame | | |
| PSAP | Prosaposin | −9 nt; exon | Coding region in | Secreted | n.d. |
| | | | frame | glycoprotein | |
| | | | | precursor | |
| PTK2 | Protein tyrosine kinase 2 | +9 nt; exon | Coding region in | Focal | CS |
| | | | frame | adhesion | |
| | | | | tyrosine | |
| | | | | kinase | |
| PTPN13 | Protein tyrosine | +57 nt; exon | Coding region in | Signalling, | ES |
| | phosphatase, non- | | frame | apoptosis | |
| | receptor type 13 (APO- | | | | |
| | 1/CD95 (Fas)- | | | | |
| | associated | | | | |
| | phosphatase) | | | | |
| RAD52 | RAD52 homolog (S. cerevisiae) | +151 nt; exon | Inclusion causes | DNA double- | ES |
| | | | reading frame | strand break | |
| | | | truncation | repair and | |
| | | | | homologous | |
| | | | | recombination | |
| SHMT1 | Serine | +117 nt; exon | Coding region in | Purine | ES |
| | hydroxymethyltransferase 1 | | frame | synthesis | |
| SLIT2 | Slit homolog 2 | −12 nt; exon | Coding region in | Migration and | CS |
| | (Drosophila) | | frame | metastasis | |
| SRP19 | Signal recognition | +112 nt; exon | Out of frame | Protein | CS |
| | particle 19 kDa | | truncating | transport to | |
| | | | | ER | |
| STIM1 | Stromal interaction | −93 nt; exon | Coding region in | Transmembrane | ES |
| | molecule 1 | | frame | protein | |
| SYK | Spleen tyrosine kinase | −69 nt; exon | Coding region in | Lamellipodia | ES |
| | | | frame | formation | |
| SYNE2 | Synaptic nuclear | −69 nt; exon | Coding region in | Nuclear | CS |
| | envelope protein 2 | | frame | anchorage to | |
| | | | | cytoskeleton | |
| TOPBP1 | Topoisomerase (DNA) II | +15 nt; alt 3′ | Coding region in | Genomic | ES |
| | binding protein 1 | | frame | stability | |
| TSSC4 | Tumor suppressing | −192 nt; intron | Coding region in | Tumor | CS |
| | subtransferable | | frame | supressor | |
| | candidate 4 | | | | |
| TUBA4A | Tubulin, alpha 1a | +223 nt; exon | Out of frame | Cytoskeleton | ES |
| (TUBA1) | | | truncating | | |
| UTRN | Utrophin | −39 nt; exon | Coding region in | Dystrophin, | CS |
| | | | frame | neuromuscular | |
| | | | | junction | |

¹ASE: alternative splicing event;
²EOC: epithelial ovarian cancer.

The present invention will be more readily understood by referring to the following examples which are given to illustrate the invention rather than to limit its scope.

### Example 1

### Mapping Splicing Events by Comprehensive RT-PCR Coverage

The gene list was obtained by a keyword search for “ovarian cancer” in NCBI Gene database. The search was performed in January 2006, and was limited to human genes with “known” RefSeq status (Nucleic Acids Res 2005 Jan. 1; 33(1): D501-D504). The 233 genes generated from this search were cross referenced with the AceView database and 182 genes showing evidence of alternative splicing were selected for this study. The exon structure of each gene was determined using AceView as a source for cDNAs and multi-exon ESTs (Thierry-Mieg & Thierry-Mieg, 2006, Genome Biol, 7 Suppl 1, S12, 1-14). The LISA automatically identifies all splice sites and generates a splicing map, as shown for the neogenin homolog 1 (NEO1) gene in FIG. 1B. Concurrently, the LISA applies a modified PRIMER3-based (Rozen & Skaletsky, 2000, Methods Mol Biol, 132: 365-386) algorithm for the automated design of PCR primers. Each gene's AceView transcript set was mapped into the LISA database and the LISA design module was used to generate a PCR experiment set. This module is a perl script which reads input sequences from the database and automatically designs PCR primers to characterize the exon structure of the gene. The overall strategy allowed designing primers for all exons in the transcript set, such that PCR experiments flanking all possible exon-exon junctions could be designed. In practice, a forward and reverse primer was designed for all internal exons, and single primers were designed for terminal exons. Primers were designed using a Primer-3 based algorithm (Rozen & Skaletsky, 2000, Methods Mol Biol, 132: 365-386) and synthesized in 96-well plates on a 25 nmole scale (IDT, Coralville, Iowa). PCR reactions were formulated to cover all constitutive splicing events with a single reaction and alternative splicing events were covered by at least 2 independent reactions. In addition, the design was such that predicted amplicon sizes fell within the 100-700 base pair range, where possible, to facilitate the data analysis. An average of 37 primers and 54 reactions were designed per gene (see Table 2).

TABLE 2

List of primer pairs for each identified gene and listed
in the sequence listing.

| | | | | |
|---|---|---|---|---|
| | | SEQ | | SEQ |
| Gene | | ID | | ID |
| name | forward sequence | NO: | reverse sequence | NO: |

| | | | | |
|---|---|---|---|---|
| AFF3 | CATGGACAGCTTCGACTTAGC | 2 | TACAGATAGACGAGGGCTGGTT | 1 |
| AFF3 | CATGGACAGCTTCGACTTAGC | 2 | GTGCCATCATCCTGTTGAGTT | 3 |
| AFF3 | ACTTAGCCCTGCTCCAGGAAT | 4 | AGAATTAAACGTGCCATCATCC | 5 |
| AFF3 | CAGGAATGGGACCTCGAGTC | 6 | GGCTCACTGAAGAGAGAGTAACTAGAA | 7 |
| AFF3 | AGCTTCGACTTAGCCCTGCT | 8 | CCATCATCCTGTTGAGTTTCTTGA | 9 |
| AFF3 | ATGGGACCTCGAGTCACTGT | 10 | CTTGTAGGGCTCACTGAAGAGAG | 3062 |
| AFF3 | TCCCACCATGGACAGCTTCG | 11 | TTAAACGTGCCATCATCCTGTTGA | 3063 |
| AFF3 | CTGCTCCAGGAATGGGACCT | 12 | GAGTAACTAGAATTAAACGTGCCATCA | 3064 |
| AFF3 | GCGGCGACGCTGACACCT | 13 | CTTTCTCGTTCTTTCCTCCGTAATGC | 3065 |
| AFF3 | CTGATCACCTGCCTTTCCAG | 14 | TGCATTTCTATCTGGTTCATAGACA | 3066 |
| AFF3 | GTTTGAAGTCGACACCCAGAG | 15 | CCTCCGTAATGCATTTCTATCTG | 16 |
| AFF3 | CCAGTTTGTCCCCCTTCC | 17 | TTCTTTCCTCCGTAATGCATTTCT | 18 |
| AFF3 | GCCTTTCCAGACCGGAGAC | 19 | GAGAGTTCATCCCCCTTGTTAG | 20 |
| AFF3 | AGAGGCCAGTTTGTCCCCCT | 21 | TCCGGTTGGAGAGTTCATCC | 22 |
| AFF3 | AGTCGACACCCAGAGGCCAG | 23 | CATCATAATTGCCTAAAGTGTTCTGG | 24 |
| AFF3 | AGACGGGCGGCAAGTTTGAA | 25 | GCCTAAAGTGTTCTGGATCCGG | 26 |
| AFF3 | CGGCAAGTTTGAAGTCGACACCC | 27 | AAAGTGTTCTGGATCCGGTTGGAG | 28 |
| AFF3 | CACCTGCCTTTCCAGACCGG | 29 | TCTGGATCCGGTTGGAGAGTTCA | 30 |
| APC | TCCTACAGCTACAGAGATTCAAGATG | 31 | TCTCTCAGTCGAGCAAGTTCC | 32 |
| APC | TGTTCAGCAGTTGGACTTAACG | 33 | TTGTGGCTTGGTTTCTCTGG | 34 |
| APC | CAAGATGTATGTTCAGCAGTTGG | 35 | CGACGTTTCTTTCGTTTCTCC | 36 |
| APC | GCAGTTGGACTTAACGTATTTCTTG | 37 | AAGTTCCTGCCGACGTTTCT | 38 |
| APC | GCTGTTGAAAATCCTACAGCTACAG | 39 | CTTCTTGGGAAACGTCATCTTC | 40 |
| APC | AAGACCATCGCAGAGGGAAG | 41 | GTCATCTTCTCAAGTGCAGCC | 42 |
| APC | AGGCGAATCCCCATAAGTAAG | 43 | TCTCAAGTGCAGCCATCTTG | 44 |
| APC | AATAAGAAGACCATCGCAGAGG | 45 | TTCGTTTCTCCTTCTTCAGGG | 46 |
| APC | CAGAGGGAAGGCGAATCC | 47 | CTGGAAATGTCATCTTCTTGGG | 48 |
| APC | TTTAAATAATAAGAAGACCATCGCAG | 49 | CCTGACCCCTCAGACATTCTTA | 50 |
| APC | AGGGAAGGCGAATCCCCATA | 51 | TGAGAGTCCTGAGTCTCTCAGTCG | 52 |
| APC | CATCGCAGAGGGAAGGCGAA | 53 | TCCTGCCGACGTTTCTTTCGTT | 54 |
| APC | AGAGGGAAGGCGAATCCCCATAAGTAA | 55 | TCGAGCAAGTTCCTGCCGAC | 56 |
| APP | GCAACCGGAACAACTTTGAC | 57 | GGGCATGTTCATTCTCATCC | 58 |
| APP | ATGTGACTGAAGGGAAGTGTGC | 59 | CGAGATACTTGTCAACGGCA | 60 |
| APP | TGACACAGAAGAGTACTGCATGG | 61 | TTCTGGAAATGGGCATGTTC | 62 |
| APP | GTGTGCCCCATTCTTTTACG | 63 | GCCTCTCTTTGGCTTTCTGG | 64 |
| APP | ACTTTGATGTGACTGAAGGGAAG | 65 | ATTCTCTCTCGGTGCTTGGC | 66 |
| APP | CGGAACAACTTTGACACAGAAG | 67 | TTGGCCTCAAGCCTCTCTTT | 68 |
| APP | TTCTTTTACGGCGGATGTGG | 69 | TTTGGCTTTCTGGAAATGGG | 70 |
| APP | AAGGGAAGTGTGCCCCATTC | 71 | ATACTTGTCAACGGCATCAGGG | 72 |
| APP | ACAACTTTGACACAGAAGAGTACTGC | 73 | GGAAATGGGCATGTTCATTCTC | 74 |
| APP | AGTACTGCATGGCCGTGTGT | 75 | CCTCAAGCCTCTTTGGCTTT | 76 |
| APP | CCCCATTCTTTTACGGCGGA | 77 | TCTCGGTGCTTGGCCTCAAG | 78 |
| APP | ATGTGGCGGCAACCGGAAC | 79 | GCATGTTCATTCTCATCCCCAGGT | 80 |
| APP | GAAGGGAAGTGTGCCCCATTCTTTTAC | 81 | TCTCGAGATACTTGTCAACGGCATCAG | 82 |
| APP | ATGGCCGTGTGTGGCAGC | 83 | CTGGGACATTCTCTCTCGGTG | 84 |
| APP | AGGTGTGCTCTGAACAAGCC | 85 | GGTACTGGCTGCTGTTGTAGG | 86 |
| APP | CGCTGGTACTTTGATGTGACTG | 87 | ATCCCCAGGTGTCTCGAGAT | 88 |
| APP | TGCCGAGCAATGATCTCC | 89 | GCATCAGGGGTACTGGCTG | 90 |
| APP | CTCTGAACAAGCCGAGACG | 91 | TTCTCATCCCCAGGTGTCTC | 92 |
| APP | AATGATCTCCCGCTGGTACTT | 93 | TCAACGGCATCAGGGGTACT | 94 |
| APP | CGAGCAATGATCTCCCGCTG | 95 | ATCAGGGGTACTGGCTGCTGTTG | 96 |
| APP | GGCCGTGCCGAGCAATGAT | 97 | ATTCTCATCCCCAGGTGTCTCGAGATA | 98 |
| APP | ATCTCCCGCTGGTACTTTGATGT | 99 | TCTGCCTCTTCCCATTCTCTC | 100 |
| APP | ACAAGCCGAGACGGGGCC | 101 | TGGCTGCTTCCTGTTCCAAA | 102 |
| APP | TGTGGAAGAGGTGGTTCGAG | 103 | GTTCTTTGCTTGACGTTCTGC | 104 |
| APP | AGAGTCTGTGGAAGAGGTGGTTC | 105 | GGCAAGTTCTTTGCTTGACG | 106 |
| APP | CAGAGAGAACCACCAGCATTG | 107 | CAGGTTTAGGCAAGTTCTTTGC | 108 |
| APP | CACCAGCATTGCCACCAC | 109 | TGCCTTCTTATCAGCTTTAGGC | 110 |
| APP | AAGAAGCCACAGAGAGAACCAC | 111 | TTCTTATCAGCTTTAGGCAAGTTCTT | 112 |
| FN1 | AATCCAAGCGGAGAGAGTCA | 113 | AACATTGGGTGGTGTCCACT | 114 |
| FN1 | TGACTATTGAAGGCTTGCAGC | 115 | ACTTCAGGTCAGTTGGTGCAG | 116 |
| FN1 | GTGGAGTATGTGGTTAGTGTCTATGC | 117 | CTTGTGGGTGTGACCTGAGTG | 118 |
| FN1 | AGAGTCAGCCTCTGGTTCAGAC | 119 | TCCAGTGAGCTGAACATTGG | 120 |
| FN1 | CTCAGAATCCAAGCGGAGAG | 121 | AGCTGAACATTGGGTGGTGT | 122 |
| FN1 | CAGAAATGACTATTGAAGGCTTGC | 123 | AGGTCAGTTGGTGCAGGAATAG | 124 |
| FN1 | CTGGTTCAGACTGCAGTAACCA | 125 | GTCACCCGCACTCGATATCC | 126 |
| FN1 | AGCCCACAGTGGAGTATGTGG | 127 | TGTGACCTGAGTGAACTTCAGG | 128 |
| FN1 | ATGTGGTTAGTGTCTATGCTCAGAATC | 129 | CTCAGGCTTGTGGGTGTGAC | 130 |
| FN1 | AGCGGAGAGAGTCAGCCTCT | 131 | TCGATATCCAGTGAGCTGAACA | 132 |
| FN1 | GAAGGCTTGCAGCCCACAGT | 133 | CTGAGTGAACTTCAGGTCAGTTGG | 134 |
| FN1 | TGTCTATGCTCAGAATCCAAGCG | 135 | GTGTCCACTGGGCGCTCA | 136 |
| FN1 | TCAGCCTCTGGTTCAGACTGCAGTA | 137 | GATATCCAGTGAGCTGAACATTGGGTG | 138 |
| FN1 | GCCCACAGTGGAGTATGTGGTTAGTGT | 139 | TTGTGGGTGTGACCTGAGTGAACTTCA | 140 |
| FN1 | CTATGCTCAGAATCCAAGCGGAGAGAG | 141 | GGGTGGTGTCCACTGGGCG | 142 |
| FN1 | GCTTGCAGCCCACAGTGGAGTA | 143 | GGGCGCTCAGGCTTGTGG | 144 |
| FN1 | GGATGACAAGGAAAGTGTCCC | 145 | GAGGTGTGCTCTCATGTTGTTC | 146 |
| FN1 | GGATGACAAGGAAAGTGTCCC | 145 | GTTGGTTAAATCAATGGATGGG | 147 |
| FN1 | GCCTGGAGTACAATGTCAGTGTT | 148 | TGGTGTCTGGACCAATGTTG | 149 |
| FN1 | TGCACTTTTGATAACCTGAGTCC | 150 | AGGTCAGTGGGAGGAGGAAC | 151 |
| FN1 | GGAAAGTGTCCCTATCTCTGATACC | 152 | CAGGAAGTTGGTTAAATCAATGG | 153 |
| FN1 | CACTGTCAAGGATGACAAGGAA | 154 | GTGGAGCCCAGGTGACAC | 155 |
| FN1 | ATCTCTGATACCATCATCCCAG | 156 | GTGAGTAACGCACCAGGAAGTT | 157 |
| FN1 | AGTGTTTACACTGTCAAGGATGACA | 158 | AGGTGACACGCATGGTGTCT | 159 |
| FN1 | ATAACCTGAGTCCCGGCCT | 160 | CAATGTTGGTGAATCGCAGG | 161 |
| FN1 | TGACAAGGAAAGTGTCCCTATCTC | 162 | CGCACCAGGAAGTTGGTTAA | 163 |
| FN1 | TTGGAAGAAGTGGTCCATGC | 164 | AATCGCAGGTCAGTGGGAG | 165 |
| FN1 | TGTCCCTATCTCTGATACCATCATCC | 166 | CACAGGTGAGTAACGCACCA | 167 |
| FN1 | TGATCAGAGCTCCTGCACTTT | 168 | TTGGTGAATCGCAGGTCAGT | 169 |
| FN1 | CTGTCAAGGATGACAAGGAAAGTGTCC | 170 | AATCAATGGATGGGGGTGGAG | 171 |
| FN1 | AGCTCCTGCACTTTTGATAACC | 172 | TGGACCAATGTTGGTGAATC | 173 |
| FN1 | TCCATGCTGATCAGAGCTCC | 174 | ACACGCATGGTGTCTGGACC | 175 |
| FN1 | GAAGAAGTGGTCCATGCTGATCA | 176 | AGCCCAGGTGACACGCATG | 177 |
| FN1 | CAAACGGCCAGCAGGGAAAT | 178 | ATGGTGTCTGGACCAATGTTGGTGAAT | 179 |
| FN1 | ATTCTTTGGAAGAAGTGGTCCATGCTG | 180 | GATGGGGGTGGAGCCCAGGT | 181 |
| FN1 | CCATAAGGCATAGGCCAAGA | 182 | TCAGTGCCTCCACTATGACG | 183 |
| FN1 | CTCTCAGACAACCATCTCATGG | 184 | TCAGTGCCTCCACTATGACG | 183 |
| FN1 | TCATCCTGTTGGCACTGATG | 185 | AACAACCTCTTCCCGAACCT | 186 |
| FN1 | GGCACTGATGAAGAACCCTTAC | 187 | GAACCTTATGCCTCTGCTGG | 188 |
| FN1 | TCATTTCATGTCATCCTGTTGG | 189 | TGCTGGTCTTTCAGTGCCTC | 190 |
| FN1 | CCCATTCCAGGACACTTCTG | 191 | TCCACTATGACGTTGTAGGTGG | 192 |
| FN1 | CAAGAAGCTCTCTCTCAGACAACC | 193 | TTGCCCACGGTAACAACCTC | 194 |
| FN1 | CCTGTTGGCACTGATGAAGAAC | 195 | CTTATGCCTCTGCTGGTCTTTC | 196 |
| FN1 | GGACACTTCTGAGTACATCATTTCA | 197 | TCTTCCCGAACCTTATGCCT | 198 |
| FN1 | CTGAGTACATCATTTCATGTCATCC | 199 | GGTCTTTCAGTGCCTCCACTAT | 200 |
| FN1 | CATTCCAGGACACTTCTGAGTACA | 201 | CACGGTAACAACCTCTTCCC | 202 |
| FN1 | AAGCTCTCTCTCAGACAACCATCTCAT | 203 | CAGAGTTGCCCACGGTAACA | 204 |
| FN1 | TTCATGTCATCCTGTTGGCACTGA | 205 | TAACAACCTCTTCCCGAACCTTATGCC | 206 |
| FN1 | ATCTCATGGGCCCCATTC | 207 | AGTGCCTCCACTATGACGTTGTAG | 208 |
| FN1 | ACAACCATCTCATGGGCCCC | 209 | TGGCACCTCTGGTGAGGC | 210 |
| FN1 | ATGGGCCCCATTCCAGGAC | 211 | ATGACGTTGTAGGTGGCACCTC | 212 |
| FN1 | CCATAAGGCATAGGCCAAGA | 182 | TGGCACTGGTAGAAGTTCCAG | 214 |
| FN1 | GAGGAACATGGTTTTAGGCG | 215 | TGTCAGAGTGGCACTGGTAGAA | 216 |
| FN1 | CAAATGATCTTTGAGGAACATGG | 217 | CTGGTGAGGCCTGTCAGAGT | 218 |
| FN1 | ATAGGCCAAGACCATACCCG | 219 | GTAGGTGGCACCTCTGGTGA | 220 |
| FN1 | ACCATACCCGCCGAATGTAG | 221 | AGGCCTGTCAGAGTGGCAC | 222 |
| FN1 | GATCTTTGAGGAACATGGTTTTAGG | 223 | CACCTCTGGTGAGGCCTGTC | 224 |
| FN1 | ACCACACCGCCCACAACG | 225 | AGAGTTGCCCACGGTAACAACCTCTTC | 226 |
| FN1 | TAAGGCATAGGCCAAGACCATAC | 227 | TAGGTTGGTTCAAGCCTTCG | 228 |
| FN1 | CACCCCCATAAGGCATAGG | 229 | TCATCCGTAGGTTGGTTCAAG | 230 |
| FN1 | AACGGCCACCCCCATAAG | 231 | AAGCACGAGTCATCCGTAGG | 232 |
| FN1 | CCAAGACCATACCCGCCGAA | 233 | CACGAGTCATCCGTAGGTTGGTT | 234 |
| FN1 | TTTAGGCGGACCACACCG | 235 | GGGTCAAAGCACGAGTCATC | 236 |
| FN1 | ACATGGTTTTAGGCGGACCA | 237 | AACTGTGTAGGGGTCAAAGCAC | 238 |
| FN1 | ACCGCCCACAACGGCCAC | 239 | GTCAAAGCACGAGTCATCCGTAGGTTG | 240 |
| FN1 | GGGCAACAAATGATCTTTGAGG | 241 | GTGTAGGGGTCAAAGCACGAGTCA | 242 |
| FN1 | GGACTCAATCCAAATGCCTC | 243 | GTTCCCACTCATCTCCAACG | 244 |
| FN1 | ATGCCTCTACAGGACAAGAAGC | 245 | TTCAGACATTCGTTCCCACTC | 246 |
| FN1 | AGACTATCACCTGTACCCACACG | 247 | CATCTCCAACGGCATAATGG | 248 |
| FN1 | ACAGGACAAGAAGCTCTCTCTCAG | 249 | CTGGCACAACAGTTTAAAGCC | 250 |
| FN1 | CCAGGGAAGATGTAGACTATCACC | 251 | CGGCATAATGGGAAACTGTG | 252 |
| FN1 | CAATCCAAATGCCTCTACAGG | 253 | ACATTCGTTCCCACTCATCTCC | 254 |
| FN1 | GAAATCCAAATTGGTCACATCC | 255 | AATGGGAAACTGTGTAGGGGTC | 256 |
| FN1 | GTCCGGGACTCAATCCAAAT | 257 | CCTAAGCACTGGCACAACAGT | 258 |
| FN1 | ACACGGTCCGGGACTCAATC | 259 | CTCCAACGGCATAATGGGAAACT | 260 |
| FN1 | ACCTGTACCCACACGGTCCG | 261 | CCCACTCATCTCCAACGGCATAA | 262 |
| FN1 | AAGATGTAGACTATCACCTGTACCCAC | 263 | GCCTGATTCAGACATTCGTTC | 264 |
| FN1 | TGGTCACATCCCCAGGGAAGAT | 265 | CAACGGCATAATGGGAAACTGTGTAGG | 266 |
| FN1 | TACCCACACGGTCCGGGACT | 267 | CTAAGCACTGGCACAACAGTTTAAAGC | 268 |
| FN1 | ACATCCCCAGGGAAGATGTAGAC | 269 | TTTAAAGCCTGATTCAGACATTCG | 270 |
| FN1 | AGGTGAGGAAATCCAAATTGG | 271 | TTCCAAAGCCTAAGCACTGG | 272 |
| FN1 | GCCGAATGTAGGTGAGGAAA | 273 | GACCACTTCCAAAGCCTAAGC | 274 |
| FN1 | CCGAATGTAGGTGAGGAAATCCAAAT | 275 | CCAAAGCCTAAGCACTGGCACAAC | 276 |
| FN1 | AATAATCAGAAGAGCGAGCCC | 277 | ACTGGGTTGCTGACCAGAAG | 278 |
| FN1 | CCCCTGATTGGAAGGAAAA | 279 | CTCAAAGATCATTTGTTGCCC | 280 |
| FN1 | AGAAGAGCGAGCCCCTGATT | 281 | TCCGCCTAAAACCATGTTCC | 282 |
| FN1 | CCTGATTGGAAGGAAAAAGACAG | 283 | GGTATGGTCTTGGCCTATGC | 284 |
| FN1 | AGCGAGCCCCTGATTGGAAG | 285 | ATTTGTTGCCCAACACTGGG | 286 |
| FN1 | ATAATCAGAAGAGCGAGCCCCTGATTG | 287 | CGTTGTGGGCGGTGTGGTC | 288 |
| FN1 | CAATTTATGTCATTGCCCTGAAG | 289 | GGAAGCTGAATACCATTTCCAG | 290 |
| FN1 | CCGGGAACCGAATATACAATT | 291 | ACCATTTCCAGTGTCATACCCA | 292 |
| FN1 | GCCCTGAAGAATAATCAGAAGAGC | 293 | TGACCAGAAGTGCCAGGAAG | 294 |
| FN1 | TGTCATTGCCCTGAAGAATAATC | 295 | GAAGTGCCAGGAAGCTGAATAC | 296 |
| FN1 | TGGAACCGGGAACCGAATAT | 297 | CTTGGCCTATGCCTTATGGG | 298 |
| FN1 | AACCGAATATACAATTTATGTCATTGC | 299 | CCATGTTCCTCAAAGATCATTTG | 300 |
| FN1 | CCTGGAACCGGGAACCGAATATACAAT | 301 | GGTATGGTCTTGGCCTATGCCTTATGG | 302 |
| FN1 | CATCAAGTATGAGAAGCCTGGG | 303 | AGGGTGGGTGACGAAAGG | 304 |
| FN1 | CAGGATTACCGGCTACATCATC | 305 | GTGACGAAAGGGGTCTTTTG | 306 |
| FN1 | CCTGGTGTCACAGAGGCTACTAT | 307 | CTACATTCGGCGGGTATGGT | 308 |
| FN1 | CACACCCAATTCCTTGCTG | 309 | GCTGAATACCATTTCCAGTGTCAT | 310 |
| FN1 | TTCCTTGCTGGTATCATGGC | 311 | CCAACACTGGGTTGCTGAC | 312 |
| FN1 | TCTCCTCCCAGAGAAGTGGTC | 313 | GCCTAAAACCATGTTCCTCAAAG | 314 |
| FN1 | CCGGCTACATCATCAAGTATGAG | 315 | CAGTGTCATACCCAGGGTGG | 316 |
| FN1 | TATGAGAAGCCTGGGTCTCCT | 317 | GGTGTGGTCCGCCTAAAAC | 318 |
| FN1 | CCCAATTCCTTGCTGGTATC | 319 | GTTGCTGACCAGAAGTGCCA | 320 |
| FN1 | CCACGTGCCAGGATTACC | 321 | AAGATCATTTGTTGCCCAACACT | 322 |
| FN1 | GCTACATCATCAAGTATGAGAAGCCTG | 323 | TCATACCCAGGGTGGGTGAC | 324 |
| FN1 | CAGAGAAGTGGTCCCTCGG | 325 | CTATGCCTTATGGGGGTGG | 326 |
| FN1 | AAGCCTGGGTCTCCTCCCAG | 327 | GTGTGGTCCGCCTAAAACCATGTT | 328 |
| FN1 | TGCACCATCCAACCTGCGTT | 329 | AGTGCCAGGAAGCTGAATACCATTTCC | 330 |
| FN1 | GCGTTTCCTGGCCACCACAC | 331 | ATACCCAGGGTGGGTGACGAAAGG | 332 |
| FN1 | CCTGGGTCTCCTCCCAGAGAAGT | 333 | TTATGGGGGTGGCCGTTGTG | 334 |
| FN1 | CCCGCCCTGGTGTCACAGAG | 335 | TTCGGCGGGTATGGTCTTGG | 336 |
| FN1 | CTGGTATCATGGCAGCCG | 337 | TTTCCTCACCTACATTCGGC | 338 |
| AXIN1 | CATGCAGTGGATCATTGAGG | 339 | ACCTTCCTCTGCGATCTTGTC | 340 |
| AXIN1 | CTCCCACCTCTTCATCCAAG | 341 | ACCTTCCTCTGCGATCTTGTC | 340 |
| AXIN1 | TCCAGACCCTTGTCCCTTGA | 342 | CAGAAGTAGTACGCCACAACGA | 343 |
| AXIN1 | CCATGAGAACTCCAGACCCTT | 344 | CCACAACGATGCTGTCACAC | 345 |
| AXIN1 | TTCATCCAAGACCCCACCAT | 346 | TCAGCAGCTCCTTGAACTGG | 347 |
| AXIN1 | ACGTCTGGAGGAGGAAGAAA | 348 | TAGCTGCCCTTTTTGGTCAG | 349 |
| AXIN1 | GGACGAGGAAGCCACAGC | 350 | AGTACGCCACAACGATGCTG | 351 |
| AXIN1 | ACCTCTTCATCCAAGACCCC | 352 | TTTGGTCAGCAGCTCCTTGA | 353 |
| AXIN1 | CAGCCCTCCCACCTCTTCAT | 354 | CCGCCCACCTTCCTCTGC | 355 |
| AXIN1 | AGCTCCCAACCCCCTAACC | 356 | GATGCTGTCACACGGCTG | 357 |
| AXIN1 | CGAGCACCCTCCAAGCAGAG | 358 | TCCTTGAACTGGCCCAGGGT | 359 |
| AXIN1 | AAAGAGAGCCAGCCGAGCAC | 360 | CCTCACCAGGGTGCGGTAG | 361 |
| AXIN1 | ACCCTTGTCCCTTGAGCACC | 362 | CACACGGCTGGGCACTCC | 363 |
| AXIN1 | ACCTCCGTGCAGCCCTCC | 364 | GAAGTAGTACGCCACAACGATGCTGTC | 365 |
| AXIN1 | TAACCCAGCTGGAGGAGGC | 366 | AGGGTGCGGTAGGGGATG | 367 |
| AXIN1 | TGAGAACTCCAGACCCTTGTCCCT | 368 | ACTCCCGCCGCCCACCTT | 369 |
| AXIN1 | AAGACCCCACCATGCCAC | 370 | CCCTTTTTGGTCAGCAGGTC | 371 |
| AXIN1 | AGCCACAGCCCCATGAGAAC | 372 | ATGGGTTCCCCGCAGAAGTA | 373 |
| AXIN1 | TTCGGGGACGAGGAAGCCAC | 374 | GCTGTCACACGGCTGGGCAC | 375 |
| AXIN1 | CGCCGACGTCTGGAGGAG | 376 | GAACTGGCCCAGGGTGACAG | 377 |
| AXIN1 | ACCATGCCACCCCACCCAG | 378 | CTTTTTGGTCAGCAGCTCCTTGAACTG | 379 |
| AXIN1 | CTCAGCTCCGGACCTCCGTG | 380 | GTAGGGGATGGGTTCCCCGC | 381 |
| AXIN1 | GGAAGAAAAGAGAGCCAGCCGAGC | 382 | CGGCCCCTCACCAGGGTG | 383 |
| AXIN1 | CAACCCCCTAACCCAGCT | 384 | ACAGTCAAACTCGTCGCTCAC | 385 |
| AXIN1 | CCCACCCAGCTCCCAACC | 386 | GTCAAACTCGTCGCTCACTTTCTT | 387 |
| AXIN1 | CCAGCCGAGCACCCTCCAAG | 388 | GACGGCCTCGTCCTCTCGAA | 389 |
| AXIN1 | CCCCCTAACCCAGCTGGAGG | 390 | AACTCGTCGCTCACTTTCTTGAAGTAG | 391 |
| AXIN1 | CTTGAGCACCCCTGGGCC | 392 | CCACCCCACAGTCAAACTCG | 393 |
| BCMP11 | AAGAGCACTGGCCAAGTCAG | 394 | CCTCCAGGTGATGAATAACCA | 395 |
| BCMP11 | AGAAACATCCAGAATACATTTCCAAC | 396 | TTTTGAGCATAAAAGAGACCTTCTTC | 397 |
| BCMP11 | TTTCCAACAAGAGCACTGGC | 398 | TTGACAATCCTCCAGGTGATG | 399 |
| BCMP11 | TCCAACAAGAGCACTGGCCAAGTC | 400 | TGACAATCCTCCAGGTGATGAATAACC | 401 |
| BCMP11 | AATACATTTCCAACAAGAGCACTGGCC | 402 | CTCCAGGTGATGAATAACCATTAATGG | 403 |
| BMP4 | CGAGAAGGCAGAGGAGGAG | 404 | CAAACTTGCTGGAAAGGCTC | 405 |
| BMP4 | GAAAGAGGAGGAAGGAAGATGC | 406 | GCCAATCTTGAACAAACTTGC | 407 |
| BMP4 | AAGAAAGAAAGCGAGGGAGG | 408 | GAAAGGCTCAGGGAAGCTG | 409 |
| BMP4 | GAAGGAAGATGCGAGAAGGC | 410 | CAGTCCATGATTCTTGACAGCC | 411 |
| BMP4 | AAGATGCGAGAAGGCAGAGG | 412 | ACAAGGCATATAATAACAGTCCATGA | 413 |
| BMP4 | AAAGCGAGGGAGGGAAAGAG | 414 | TTGACAGCCAATCTTGAACAAA | 415 |
| BMP4 | GAGGAGGAAGGAAGATGCGAGAAG | 416 | TTGCTGGAAAGGCTCAGGGAA | 417 |
| BMP4 | AGCCCGGCCCGGAAGCTAG | 418 | ATGGCTCGCGCCTCCTAGC | 419 |
| BMP4 | GGAAGGAGCGCGGAGCCC | 420 | CTAGCATGGCTCGCGCCTCC | 421 |
| BTC | ACCACCACACAATCAAAGCG | 422 | TTACGACGTTTCCGAAGAGG | 423 |
| BTC | CCCAAGCAATACAAGCATTACTG | 424 | CCCAGAGTTTCCATTTCTTCTTC | 425 |
| BTC | ACTGCATCAAAGGGAGATGC | 426 | GGAGTTATATCTTTACCCAGAGTTTCC | 427 |
| BTC | ACAAGCATTACTGCATCAAAGG | 428 | TCTTTACCCAGAGTTTCCATTTCT | 429 |
| BTC | AAAGCGGAAAGGCCACTTCT | 430 | TGTCTCTTCAATATCTTCATTGATAGG | 431 |
| BTC | CAAAGGGAGATGCCGCTT | 432 | CATTGATAGGAGTTATATCTTTACCCA | 433 |
| BTC | GCCACTTCTCTAGGTGCCCCAAG | 434 | TCTTCTTTCTTTTACGACGTTTCCGA | 435 |
| BTC | AGCAGACGCCCTCCTGTGT | 436 | TTCCTGAGACACATTCTGTCCA | 437 |
| BTC | GATGCCGCTTCGTGGTGG | 438 | CAAATGAGCAAGGCACTTTGC | 439 |
| BTC | AAGCAATACAAGCATTACTGCATCAAA | 440 | CACCAACCTGGAGGTAACTTCA | 441 |
| BTC | TTCGTGGTGGCCGAGCAGAC | 442 | GGCACTTTGCAGCTTGCCAC | 443 |
| BTC | AAGCATTACTGCATCAAAGGGAGATGC | 444 | TTGCAGCTTGCCACCAACCT | 445 |
| BTC | AGGGAGATGCCGCTTCGTGG | 446 | GAGCAAGGCACTTTGCAGCTTG | 447 |
| BTC | ACAATCAAAGCGGAAAGGCCACTT | 448 | GCTTGCCACCAACCTGGAGG | 449 |
| BTC | AAAGCGGAAAGGCCACTTCTCTAGGTG | 450 | GTCCATTTTCAAATGAGCAAGGCACT | 451 |
| BTC | AGACCCTGAGGAAAACTGTGC | 452 | CCTGGAGGTAACTTCATAGCCT | 453 |
| BTC | TCCTCTGTGGAGACCCTGAG | 454 | AGCTGTTTTCCTGAGACACATTC | 455 |
| BTC | TGTGGAGACCCTGAGGAAAA | 456 | GTCTACTAGCTGTTTTCCTGAGACAC | 457 |
| C11 | AGCCATGGACAACTGTTTGG | 458 | CATGCTCTGATATTTGATAGCTGC | 459 |
| C11 | AGCCATGGACAACTGTTTGG | 458 | GACTCGCCTCTGTGATAACGAT | 461 |
| C11 | TCTAGAAGTGCTGGAGAGGGC | 462 | AACGATAGACATGGGTTGCC | 463 |
| C11 | CAAGGCTGGCTCTAGAAGTGC | 464 | GACCATTCCCTATGTCCAAGC | 465 |
| C11 | CCCACCTAGAGAAACAGCCG | 466 | ATGTCCAAGCACATGTGCAG | 467 |
| C11 | GTTCAGCAAGGCTGGCTCTA | 468 | CTTCCTCTGGCACAGATGAAT | 469 |
| C11 | CCTGCAGCGTTCAGCAAG | 470 | TCCCTATGTCCAAGCACATG | 471 |
| C11 | CTGGAGAGGGCCAAGAGGAG | 472 | ACATGTGCAGCTTCGACTCG | 473 |
| C11 | TGAATGGGGTGGACCGAC | 474 | CTCTGTGATAACGATAGACATGGG | 475 |
| C11 | ACCGACGTTCCCTGCAGC | 476 | GTTGCCCCTCCTTCCTCTG | 477 |
| C11 | AAGAGGAGGGCGGTGGACTG | 478 | CAAGCACATGTGCAGCTTCG | 479 |
| C11 | GTCCCAAAGGCTGCATGG | 480 | TAGACATGGGTTGCCCCTCC | 481 |
| C11 | ACGTTCCCTGCAGCGTTCAG | 482 | CCCTCCTTCCTCTGGCACA | 483 |
| C11 | GACTGGCATGCCCTGGAG | 484 | TCCTCTGGCACAGATGAATAATATTT | 485 |
| C11 | ATGCCCTGGAGCGTCCCAAAG | 486 | ACCATTCCCTATGTCCAAGCACATGTG | 487 |
| C11 | GCAGCGTTCAGCAAGGCTGG | 488 | GATAACGATAGACATGGGTTGCCCCTC | 489 |
| C11 | CTAGAGAAACAGCCGGCAGC | 490 | CATGCTCTGATATTTGATAGCTGC | 459 |
| C11 | GGAGCGTCCCAAAGGCTGC | 492 | TGCCTCCAGGACCAAGGGAT | 493 |
| C11 | AGGCGCCCCACCTAGAGAAA | 494 | CCAGGACCAAGGGATGTCTTT | 495 |
| C11 | AGGGCGGTGGACTGGCATG | 496 | TGGGATGCCTCTGGAGCATG | 497 |
| C11 | GAAGTGCTGGAGAGGGCCAAGAG | 498 | TCTGATATTTGATAGCTGCCTCCAGG | 499 |
| C11 | CTGCATGGGGGTCCTTGC | 500 | GCCTCTGGAGCATGCTCTGATAT | 501 |
| C11 | AAAGGCTGCATGGGGGTC | 502 | ATAGCTGCCTCCAGGACCAA | 503 |
| C11 | GGTGGACCGACGTTCCCT | 504 | TCTGGAGCATGCTCTGATATTTGATAG | 505 |
| C11 | AGCGCTGAATGGGGTGGAC | 506 | GTAGAGGTCCTTAGAGATGTTCTCAGC | 507 |
| C11 | TGTTCAAGGCCAAGGTGAAG | 508 | TCAAAGAAGTAGGACCGAGAGG | 509 |
| C11 | TGTTCAAGGCCAAGGTGAAG | 508 | CAGTAGCTCCCACACGAAGAG | 510 |
| C11 | GCCAAGGTGAAGCGTCGG | 511 | AGGGTGGTGGGCAGTAGCTC | 512 |
| C11 | GGAGATGAGCCGAGGCTT | 513 | GGAAGAAGCCCACCAGCAG | 514 |
| C11 | AAGCGTCGGCCGGAGATGAG | 515 | CCCACCAGCAGGGTGGTGG | 516 |
| C11 | TCACCTTGACGCTTATGAACC | 517 | AGGTAGCCTTTGTTCCCCAG | 518 |
| C11 | TGCAGTTCTTCACCTTGACG | 519 | TTGTTCCCCAGGTCATTCAC | 520 |
| C11 | CGCTTATGAACCTCTACTTTGCC | 521 | GCCAAATACCAGGTAGCCTTTG | 522 |
| C11 | CGTCTGCCTGCAGTTCTTCA | 523 | CCAGGTCATTCACCAGGTCC | 524 |
| C11 | CACCTTGACGCTTATGAACCTCTACTT | 525 | GTAGCCTTTGTTCCCCAGGTCATT | 526 |
| C11 | ACTCCCTGTTCGTCATCTGC | 527 | TTGTTCCCCAGGTCATTCAC | 520 |
| C11 | CGCGCTGTCTCTTGCTGC | 529 | GCCAAATACCAGGTAGCCTTTG | 522 |
| C11 | GCGCCCTCCACTAGCATCTA | 531 | GGAAGAAGCCCACCAGCAG | 514 |
| C11 | TGAGCGACTCCCTGTTCGTC | 533 | CCAGGTCATTCACCAGGTCC | 524 |
| C11 | TCTTGCTGCCTGCCTCTG | 535 | CAGTAGCTCCCACACGAAGAG | 510 |
| C11 | GTTCGTCATCTGCGCGCT | 537 | AGGTAGCCTTTGTTCCCCAG | 518 |
| C11 | CTCTGCCTCGTCGCCAGG | 539 | AGGGTGGTGGGCAGTAGCTC | 512 |
| C11 | GTCATCTGCGCGCTGTCTCTT | 541 | GTAGCCTTTGTTCCCCAGGTCATT | 526 |
| C11 | GCCCTCCACTAGCATCTACCTGGA | 543 | CCCACCAGCAGGGTGGTGG | 516 |
| C11 | CTGTCTCTTGCTGCCTGCCT | 545 | GGATGAGGCCAAATACCAGG | 546 |
| C11 | GCCTGCCTCTGCCTCGTC | 547 | CTCCCACACGAAGAGGATGAG | 548 |
| C11 | GCATCTACCTGGAGGCCAAG | 549 | GTGCACCCGGAAGAAGCC | 550 |
| C11 | GTCCTGGTGAGCGACTCCCT | 551 | CGAAGAGGATGAGGCCAAAT | 552 |
| C11 | CTGCTGCTTGTCCGCGTC | 553 | AATACCAGGTAGCCTTTGTTCCCC | 554 |
| C11 | TGGGCCCTGCTGCTTGTC | 555 | GTGGGCAGTAGCTCCCACAC | 556 |
| C11 | TGTGCTGTGCTCTCCCATC | 557 | CAGTAGCTCCCACACGAAGAG | 510 |
| C11 | CTTGTCCGCGTCCTGGTGAG | 559 | GTCCTGTGGGGGCCGGTG | 560 |
| C11 | CTTGCTGCCTGCCTCTGCCT | 561 | CACCCGGAAGAAGCCCACCA | 562 |
| C11 | GTGCTGTGTGCTGTGCTCTC | 563 | ATTGAGGATGTGGCTGGTGC | 564 |
| C11 | TCTTTCTGCTGGTGAACGTG | 565 | CTGCCCATTGAGGATGTGG | 566 |
| C11 | ACAGCCCTGGGCCCTGCT | 567 | AGGCAAAGACCTGCCCATTG | 568 |
| C11 | GTGCTCTCCCATCGGCGC | 569 | CAAAGACCTGCCCATTGAGGATG | 570 |
| C11 | AGTTCTTCACCTTGACGCTTATG | 571 | CTCCCACACGAAGAGGATGAG | 548 |
| C11 | GGCTTCTCTACTGCTGCCC | 573 | GGATGAGGCCAAATACCAGG | 546 |
| C11 | CCTTGCCCTTCTGGCTTCTCTA | 575 | AATACCAGGTAGCCTTTGTTCCCC | 554 |
| C11 | CCTTCTGGCTTCTCTACTGCTG | 577 | CGAAGAGGATGAGGCCAAAT | 552 |
| C11 | TCTACTGCTGCCCCGTCTG | 579 | GTGGGCAGTAGCTCCCACAC | 556 |
| C11 | AGATACTCCCCGCGCCAAC | 581 | GTGCACCCGGAAGAAGCC | 550 |
| C11 | CTGCCCCGTCTGCCTGCA | 583 | CACCCGGAAGAAGCCCACCA | 562 |
| C11 | TGGGGCCCTTGCCCTTCTG | 585 | GTCCTGTGGGGGCCGGTG | 560 |
| CCNE1 | CACAGGGAGACCTTTTACTTGG | 587 | TCAAGGCAGTCAACATCCAG | 588 |
| CCNE1 | ATCCTCCAAAGTTGCACCAG | 589 | TCAAGGCAGTCAACATCCAG | 588 |
| CCNE1 | GCACCAGTTTGCGTATGTGA | 590 | ATGATACAAGGCCGAAGCAG | 591 |
| CCNE1 | GACAGATGGAGCTTGTTCAGG | 592 | GATGACGAGAAATGATACAAGGC | 593 |
| CCNE1 | TTGCGTATGTGACAGATGGAG | 594 | GCCGAAGCAGCAAGTATACC | 595 |
| CCNE1 | GGAGCTTGTTCAGGAGATGAAA | 596 | TGCATCAATTCAGATGACGAG | 597 |
| CCNE1 | CCAAAGTTGCACCAGTTTGC | 598 | ACCATAAGGAAATTCAAGGCAG | 599 |
| CCNE1 | GAAATCTATCCTCCAAAGTTGCAC | 600 | GTCAACATCCAGGACACAGAGA | 601 |
| CCNE1 | GGAGATGAAATTCTCACCATGG | 602 | TGAAACCTTTTGCATCAATTCA | 603 |
| CCNE1 | GTATGTGACAGATGGAGCTTGTTC | 604 | TCAATTCAGATGACGAGAAATGAT | 605 |
| CCNE1 | ACCAGTTTGCGTATGTGACAGATG | 606 | TACAAGGCCGAAGCAGCAAGTA | 607 |
| CCNE1 | AAAGTTGCACCAGTTTGCGTATGT | 608 | AGGAAATTCAAGGCAGTCAACA | 609 |
| CCNE1 | TGTTCAGGAGATGAAATTCTCACC | 610 | ACCTTTTGCATCAATTCAGATGAC | 611 |
| CCNE1 | CTATCCTCCAAAGTTGCACCAGTTTGC | 612 | CGAGAAATGATACAAGGCCGAAGC | 613 |
| CCNE1 | AGATGAAATTCTCACCATGGAATTAAT | 614 | CGAAGCAGCAAGTATACCATAAGG | 615 |
| CCNE1 | CACAGGGAGACCTTTTACTTGG | 587 | CCAGGACACAGAGATCCAACA | 617 |
| CCNE1 | GGGAGACCTTTTACTTGGCACAAG | 618 | AAGGCAGTCAACATCCAGGACACA | 619 |
| CCNE1 | TTATTTATTGCAGCCAAACTTGAG | 620 | ACACAGTTCTCTATGTCGCACC | 621 |
| CCNE1 | ACAGCTTATTGGGATTTCATCTTT | 622 | CCACTTGACACAGTTCTCTATGTCG | 623 |
| CCNE1 | ACTCTTTTACAGCTTATTGGGATTTC | 624 | TGGAACCATCCACTTGACAC | 625 |
| CCNE1 | GGCGACACAAGAAAATGTTGT | 626 | TAACCATGGCAAATGGAACC | 627 |
| CCNE1 | TTCTTTGACCGGTATATGGCG | 628 | ACAGTTCTCTATGTCGCACCACTGATA | 629 |
| CCNE1 | CGGTATATGGCGACACAAGA | 630 | CCCTTATAACCATGGCAAATGG | 631 |
| CCNE1 | TTTGACCGGTATATGGCGACACAA | 632 | AATGGAACCATCCACTTGACACAGTTC | 633 |
| CCNE1 | GGTATATGGCGACACAAGAAAATGTT | 634 | CTTATAACCATGGCAAATGGAACCATC | 635 |
| CCNE1 | TCTGCAGCCAAAAATGCGAG | 636 | GAAATTCAAGGCAGTCAACATCCAGGA | 637 |
| CHEK2 | CAGCTCTCAATGTTGAAACAGAA | 638 | TCTGGCTTTAAGTCACGGTGT | 639 |
| CHEK2 | CAGCTCTCAATGTTGAAACAGAA | 638 | ACAGCCAAGAGCATCTGGTAA | 641 |
| CHEK2 | GGAAGTTTGCTATTGGTTCAGC | 642 | GCTTCTTTCAGGCGTTTATTCC | 643 |
| CHEK2 | GAAAGTAGCCATAAAGATCATCAGC | 644 | CACTTTGTCAAACAGCTCTCCC | 645 |
| CHEK2 | CCTGTGGAGAGGTAAAGCTGG | 646 | CAGGTAGCTTCTTTCAGGCG | 647 |
| CHEK2 | CATCAGCAAAAGGAAGTTTGC | 648 | GCGTTTATTCCCCACCACTT | 649 |
| CHEK2 | TAAAGCTGGCTTTCGAGAGG | 650 | CCCACCACTTTGTCAAACAG | 651 |
| CHEK2 | GCAAAAGGAAGTTTGCTATTGG | 652 | TTATTCCCCACCACTTTGTCAA | 653 |
| CHEK2 | CCATAAAGATCATCAGCAAAAGG | 654 | AACAGCTCTCCCCCTTCCAT | 655 |
| CHEK2 | CTATTGGTTCAGCAAGAGAGGC | 656 | TGGTAAAAATAGAGCTTGCAGGTAGC | 657 |
| CHEK2 | GCTGGCTTTCGAGAGGAAAACA | 658 | GCAGGTAGCTTCTTTCAGGCGTTTATT | 659 |
| CHEK2 | TGGAGAGGTAAAGCTGGCTTTCG | 660 | TTTCAGGCGTTTATTCCCCACCAC | 661 |
| CHEK2 | TGGCTTTCGAGAGGAAAACATGTAAGA | 662 | TTGTCAAACAGCTCTCCCCCTTCC | 663 |
| CHEK2 | TGGTGCCTGTGGAGAGGTAA | 664 | TCTGGCTTTAAGTCACGGTGT | 639 |
| CHEK2 | CATGTAAGAAAGTAGCCATAAAGATCA | 666 | GACAGTCCTCTTCTTGAGATGACA | 667 |
| CHEK2 | AAGTTTGCTATTGGTTCAGCAAGAGAG | 668 | CACGGTGTATAATACCGTTTTCATG | 669 |
| CHEK2 | GCACTGTCACTAAGCAGAAATAAAG | 670 | CTTGGAGTGCCCAAAATCAG | 671 |
| CHEK2 | TGATCAGTCAGTTTATCCTAAGGC | 672 | CTTGGAGTGCCCAAAATCAG | 671 |
| CHEK2 | TGAAATTGCACTGTCACTAAGCA | 673 | AATCTTGGAGTGCCCAAAATCAGTAAT | 674 |
| DNMT3B | CAAGAGGGACATCTCACGGT | 675 | AGTGCACAGGAAAGCCAAAG | 676 |
| DNMT3B | GCCATCAAAGTTTCTGCTGC | 677 | AGTGCACAGGAAAGCCAAAG | 676 |
| DNMT3B | AATCCAGTGATGATTGATGCC | 678 | TTGGACACGTCTGTGTAGTGC | 679 |
| DNMT3B | AGTTTCTGCTGCTCACAGGG | 680 | AAGAGGTGTCGGATGACAGG | 681 |
| DNMT3B | CGATACTTCTGGGGCAACCTA | 682 | TGGCTGGAACTATTCACATGC | 683 |
| DNMT3B | TCACAGGGCCCGATACTTCT | 684 | CATGCAAAGTAGTCCTTCAGAGG | 685 |
| DNMT3B | CATCAAAGTTTCTGCTGCTCACAG | 686 | TCAGGAATCACACCTCCTGG | 687 |
| DNMT3B | AACCTACCCGGGATGAACAG | 688 | TATGACCCACACAGCTGAGG | 689 |
| DNMT3B | GTGATGATTGATGCCATCAAAG | 690 | CGTCTGTGTAGTGCACAGGAAA | 691 |
| DNMT3B | ATTGATGCCATCAAAGTTTCTGC | 692 | AATCACACCTCCTGGGTCCT | 693 |
| DNMT3B | GCTGCTCACAGGGCCCGATA | 694 | GGCTGGAACTATTCACATGCAAAGTAG | 695 |
| DNMT3B | TCTGGGGCAACCTACCCGG | 696 | TTCAGAGGGGCGAAGAGGTG | 697 |
| DNMT3B | GGGCCCGATACTTCTGGGGC | 698 | CCTGGGGATGCCTTCAGGAAT | 699 |
| DNMT3B | CAAGAGGGACATCTCACGGT | 675 | ATGACAGGCACGCTCCAG | 701 |
| DNMT3B | CCGTTCTTCTGGATGTTTGAG | 702 | CCATGTTGGACACGTCTGTG | 703 |
| DNMT3B | TGAATTACTCACGCCCCAAG | 704 | AGGACCTTCCCAGCAGCTTC | 705 |
| DNMT3B | GTTGTAGCCATGAAGGTTGGC | 706 | GGGCGAAGAGGTGTCGGAT | 707 |
| DNMT3B | GGATGTTTGAGAATGTTGTAGCC | 708 | TAGTCCTTCAGAGGGGCGAA | 709 |
| DNMT3B | ACATCTCACGGTTCCTGGAG | 710 | CTATTCACATGCAAAGTAGTCCTTCA | 711 |
| DNMT3B | CACCTGCTGAATTACTCACGC | 712 | ACGGCCCATGTTGGACAC | 713 |
| DNMT3B | ATGAAGGTTGGCGACAAGAG | 714 | GGGCCTGGCTGGAACTATTC | 715 |
| DNMT3B | TGAGAATGTTGTAGCCATGAAGG | 716 | CACGCTCCAGGACCTTCC | 717 |
| DNMT3B | CGTTCTTCTGGATGTTTGAGAATGTTG | 718 | AGCTTCTGGCGGGCACCAC | 719 |
| DNMT3B | GGTTGGCGACAAGAGGGACAT | 720 | GTCGGATGACAGGCACGCTC | 721 |
| DNMT3B | GCCCCAAGGAGGGTGATGAC | 722 | GTGTAGTGCACAGGAAAGCCAAAGATC | 723 |
| DNMT3B | GTAGCCATGAAGGTTGGCGACAAG | 724 | ACAGGCACGCTCCAGGACCT | 725 |
| DNMT3B | AGAGGGACATCTCACGGTTCCTGG | 726 | GCTCTGCCACACACCCCAGT | 727 |
| DNMT3B | GGCCGGCTCTTCTTCGAATT | 728 | GCACCACGGCCCATGTTG | 729 |
| DNMT3B | AGGGTGATGACCGGCCGTT | 730 | GCTCCAGGACCTTCCCAGCA | 731 |
| DNMT3B | ACCGGCCGTTCTTCTGGAT | 732 | AAAGTAGTCCTTCAGAGGGGCGAAGAG | 733 |
| DNMT3B | TACTCACGCCCCAAGGAGGG | 734 | GTCCTGGCTCTGCCACACAC | 735 |
| DNMT3B | CATCAAAGTTTCTGCTGCTCAC | 736 | TCAGGAATCACACCTCCTGG | 687 |
| DNMT3B | TTCTTCGAATTTTACCACCTGC | 738 | AGTGCACAGGAAAGCCAAAG | 676 |
| DNMT3B | TTTTACCACCTGCTGAATTACTCA | 740 | TCCTGGGTCCTGGCTCTG | 741 |
| DNMT3B | CGGCTCTTCTTCGAATTTTACCAC | 742 | ACACCCCAGTGGGCTTGG | 743 |
| DNMT3B | CTTCGAATTTTACCACCTGCTGAATTA | 744 | CAGTGGGCTTGGGGCCTG | 745 |
| DNMT3B | TACAGGCCGGCTCTTCTTCGAATTTTA | 746 | GCTTGGGGCCTGGCTGGA | 747 |
| DNMT3B | TTTCTGCTGCTCACAGGGC | 748 | AAGAGGTGTCGGATGACAGG | 681 |
| DNMT3B | CTTCGAATTTTACCACCTGCTGAATTA | 744 | GCTTGGGGCCTGGCTGGA | 747 |
| DNMT3B | AATCCAGTGATGATTGATGCC | 678 | GTTTGATCGAGTTCGACTTGG | 753 |
| DNMT3B | AGTTTCTGCTGCTCACAGGG | 680 | CTTCTTTGCCATTCATGACAAC | 755 |
| DNMT3B | GCCATCAAAGTTTCTGCTGC | 677 | ACCACAAAACATCTTCTTTGCC | 757 |
| DNMT3B | TCACAGGGCCCGATACTTCT | 684 | CCATTCATGACAACAGGGAAA | 759 |
| DNMT3B | AACCTACCCGGGATGAACAG | 688 | TTTCGAGCTCAGTGCACCAC | 761 |
| DNMT3B | CGATACTTCTGGGGCAACCTA | 682 | AAACATCTTCTTTGCCATTCATG | 763 |
| DNMT3B | GTGATGATTGATGCCATCAAAG | 690 | TGGTTTTTCCCCTGTTTGATC | 765 |
| DNMT3B | CATCAAAGTTTCTGCTGCTCACAG | 686 | CATGACAACAGGGAAAAGTTGG | 767 |
| DNMT3B | ATTGATGCCATCAAAGTTTCTGC | 692 | CTCAGTGCACCACAAAACATCT | 769 |
| DNMT3B | GCTGCTCACAGGGCCCGATA | 694 | GACAACAGGGAAAAGTTGGTTTTTCC | 771 |
| DNMT3B | GGGCCCGATACTTCTGGGGC | 698 | AGGGAAAAGTTGGTTTTTCCCCTGTTT | 773 |
| DNMT3B | CCGTTCTTCTGGATGTTTGAG | 702 | TCGACTTGGTGGTTATTGTCTG | 775 |
| DNMT3B | GTTGTAGCCATGAAGGTTGGC | 706 | TCCCCTGTTTGATCGAGTTC | 777 |
| DNMT3B | GGATGTTTGAGAATGTTGTAGCC | 708 | TCGAGTTCGACTTGGTGGTT | 779 |
| DNMT3B | CACCTGCTGAATTACTCACGC | 712 | GACTTGGTGGTTATTGTCTGTACTTTC | 781 |
| DNMT3B | GGCCGGCTCTTCTTCGAATT | 728 | ATCGAGTTCGACTTGGTGGTTATTGTC | 783 |
| DNMT3B | GTAGCCATGAAGGTTGGCGACAAG | 724 | TTTCCCCTGTTTGATCGAGTTCGACTT | 785 |
| DNMT3B | ACATCTCACGGTTCCTGGAG | 710 | AAGAGGTGTCGGATGACAGG | 681 |
| DNMT3B | ACCGGCCGTTCTTCTGGAT | 732 | CCATGTTGGACACGTCTGTG | 703 |
| DNMT3B | AGAGGGACATCTCACGGTTCCTGG | 726 | GCACCACGGCCCATGTTG | 729 |
| DNMT3B | TTTTACCACCTGCTGAATTACTCA | 740 | TTGGACACGTCTGTGTAGTGC | 679 |
| DNMT3B | CGGCTCTTCTTCGAATTTTACCAC | 742 | ACGGCCCATGTTGGACAC | 713 |
| DNMT3B | TACTCACGCCCCAAGGAGGG | 734 | GACACGTCTGTGTAGTGCACAGGA | 797 |
| DNMT3B | CTTCGAATTTTACCACCTGCTGAATTA | 744 | ACAGGCACGCTCCAGGACCT | 725 |
| DNMT3B | GTTGTAGCCATGAAGGTTGGCG | 800 | ACAGGCACGCTCCAGGACCT | 799 |
| DNMT3B | TACTCACGCCCCAAGGAGGG | 734 | GGGCGAAGAGGTGTCGGAT | 707 |
| DNMT3B | CCGTGATAGCATCAAAGAATGA | 804 | TGGCTGGAACTATTCACATGC | 683 |
| DNMT3B | ATAAACTCGAGCTGCAGGACTG | 806 | AAGAGGTGTCGGATGACAGG | 681 |
| DNMT3B | GGACTGCTTGGAATACAATAGGA | 808 | TCAGGAATCACACCTCCTGG | 687 |
| DNMT3B | CATCAAAGAATGATAAACTCGAGC | 810 | CATGCAAAGTAGTCCTTCAGAGG | 685 |
| DNMT3B | CTTGGAATACAATAGGATAGCCAAG | 812 | TATGACCCACACAGCTGAGG | 689 |
| DNMT3B | AGCTGCAGGACTGCTTGGAATA | 814 | TTCAGAGGGGCGAAGAGGTG | 697 |
| DNMT3B | GTGATAGCATCAAAGAATGATAAACTC | 816 | AATCACACCTCCTGGGTCCT | 693 |
| DNMT3B | AGAATGATAAACTCGAGCTGCAGG | 818 | TCCTGGGTCCTGGCTCTG | 741 |
| DNMT3B | AGTTTCTGCTGCTCACAGGG | 680 | CTATTCACATGCAAAGTAGTCCTTCA | 711 |
| DNMT3B | AACCTACCCGGGATGAACAG | 688 | CCATGTTGGACACGTCTGTG | 703 |
| DNMT3B | CGATACTTCTGGGGCAACCTA | 682 | GGGCCTGGCTGGAACTATTC | 715 |
| DNMT3B | CATCAAAGTTTCTGCTGCTCACAG | 686 | ACGGCCCATGTTGGACAC | 713 |
| DNMT3B | TCACAGGGCCCGATACTTCT | 684 | AGGACCTTCCCAGCAGCTTC | 705 |
| DNMT3B | ATTGATGCCATCAAAGTTTCTGC | 692 | ATGACAGGCACGCTCCAG | 701 |
| DNMT3B | TCTGGGGCAACCTACCCGG | 696 | GGGCGAAGAGGTGTCGGAT | 707 |
| DNMT3B | GCGACAAGAGGGACATCTCACG | 834 | AGCTTCTGGCGGGCACCAC | 719 |
| DNMT3B | ACATCTCACGGTTCCTGGAG | 710 | CTGGCTCTGCCACACACC | 837 |
| DNMT3B | CAAGAGGGACATCTCACGGTTCCT | 838 | AAAGTAGTCCTTCAGAGGGGCGAAGAG | 733 |
| DNMT3B | ATGAAGGTTGGCGACAAGAG | 714 | ACACACCCCAGTGGGCTT | 841 |
| FANCA | AACCTGAAGCTGATGCTCTTTC | 842 | TATCCTCATTTCCTGTGCGG | 843 |
| FANCA | TTACCAAGACTGGTTACACCTGG | 844 | TATCCTCATTTCCTGTGCGG | 843 |
| FANCA | AACCTGAAGCTGATGCTCTTTC | 842 | CTGCAATCTGGAAATAATATCCTCA | 846 |
| FANCA | CTGGAGCTGGAAATTCAACC | 847 | GAAATAATATCCTCATTTCCTGTGC | 848 |
| FANCA | AAGACTGGTTACACCTGGAGCTGG | 849 | CATTTCCTGTGCGGCCACC | 850 |
| FANCA | GTTACACCTGGAGCTGGAAATTC | 851 | GCCACCAAAGACCAAATCAG | 852 |
| FANCA | ACCTGGAGCTGGAAATTCAACCTGAAG | 853 | TCCTGTGCGGCCACCAAAGAC | 854 |
| FANCA | ACCCTTGCACCTTCCTTCTG | 855 | TCTGAGTGGTCATAACTCCTTGAG | 856 |
| FANCA | CGAGAGGTGTTGAAAGAGGAAG | 857 | CCAAATCAGAATTTTCTGAGTGG | 858 |
| FANCA | CTCTTTCTGAGGAGGACGTAGC | 859 | AGAATTTTCTGAGTGGTCATAACTCC | 860 |
| FANCA | TGATGCTCTTTCAGATACTGAACG | 861 | GAGAGGCACTATGAGGTCTTGC | 862 |
| FANCA | TGAAGCTGATGCTCTTTCAGATAC | 863 | CAAAGAGGAAGTGCTCCTGG | 864 |
| FANCA | GACACACAGAACCTTCCGAGA | 865 | AGCTCCAGGTCAGCTACCATC | 866 |
| FANCA | CCCTCTCTCTCTGGACACACA | 867 | TATGAGGTCTTGCTGCAGCTC | 868 |
| FANCA | AGGTGTTGAAAGAGGAAGATGTTC | 869 | AAATCTCAAAGAGGAAGTGCTCC | 870 |
| FANCA | AGAACCTTCCGAGAGGTGTTG | 871 | GTGTGGCCGAGAGGCACTAT | 872 |
| FANCA | CTCTCTGGACACACAGAACCTTC | 873 | GTCTTGCTGCAGCTCCAGGT | 874 |
| FANCA | ACCTTCCGAGAGGTGTTGAAAGAG | 875 | AAGGGGTGTGGCCGAGAG | 876 |
| FANCA | CAGACTGGCAGAGAGCTGC | 877 | CTCCTGGGAAGGGGTGTG | 878 |
| FANCA | GAGCTGCCCTCTCTCTCTGG | 879 | AAGTGCTCCTGGGAAGGG | 880 |
| FANCA | ACTGGCAGAGAGCTGCCCTCTC | 881 | GTGGCCGAGAGGCACTATGAGGTC | 882 |
| FANCA | CTTCTGCAGACTGGCAGAGAG | 883 | TGCGGAAAATCTCAAAGAGG | 884 |
| FANCA | TGGAGACCCTTGCACCTTC | 885 | CCGTCTGCGGAAAATCTCAA | 886 |
| FANCA | TTTCCTGGAGACCCTTGCAC | 887 | CTGGAGCCGTCTGCGGAAA | 888 |
| FANCL | CTGTGTTTCTCCGGACTTCG | 889 | ATGGTACTGAAGCAGGTATCCG | 890 |
| FANCL | CCGTGTATGAGGGATTCATCTC | 891 | TTTGCCTTCAACTTGAGAGTGA | 892 |
| FANCL | GGTCGAAAACCGTGTATGAGG | 893 | CAACTTGAGAGTGATTAAATGCTCTC | 894 |
| FANCL | AGAGCTTTTCTGTGTTTCTCCG | 895 | TTAACTTGATGGTACTGAAGCAGG | 896 |
| FANCL | ATGTGCAGGACCCAGCAG | 897 | TGCTCTCTACCAGAAGCATCTTC | 898 |
| FANCL | ACCCAGCAGGTCTAGAGCTTT | 899 | GCAGGTATCCGCATACACAAG | 900 |
| FANCL | GTGACGGAAGCGAGCCTGTT | 901 | GCATCTTCTGCTTTTAACTTGATGG | 902 |
| FANCL | CCTGCTTCTGCCCCAGAAC | 903 | GAGAGTGATTAAATGCTCTCTACCAGA | 904 |
| FANCL | GAGCCTGTTGCGCCAGTG | 905 | TTCTGCTTTTAACTTGATGGTACTGAA | 906 |
| FANCL | GCAGGTCTAGAGCTTTTCTGTGTT | 907 | CTACCAGAAGCATCTTCTGCTTT | 908 |
| FANCL | CCGGACTTCGAGCCATGG | 909 | ACTGAAGCAGGTATCCGCATACA | 910 |
| FANCL | AGGGATTCATCTCGGCTCAG | 911 | GAGGCACAAAATGGAACAGG | 912 |
| FANCL | AGAACCGGTCGAAAACCGT | 913 | AAATAATCTGGTGATTCTGCAGG | 914 |
| FANCL | GCAGGACCCAGCAGGTCTAG | 915 | TGGAACAGGAAAATCCACAAA | 916 |
| FANCL | CATGGCGGTGACGGAAGC | 917 | GAGGTGTCCAGGAGGCACAA | 918 |
| FANCL | CGAAAACCGTGTATGAGGGATTCA | 919 | GGCACAAAATGGAACAGGAAAATC | 920 |
| FANCL | GTGTATGAGGGATTCATCTCGGCTCAG | 921 | TGTCCAGGAGGCACAAAATGGA | 922 |
| FANCL | CAGTGCCCCCTGCTTCTG | 923 | CAGGAAAATCCACAAAATAATCTGG | 924 |
| FANCL | TCTGCCCCAGAACCGGTC | 925 | ATCCACAAAATAATCTGGTGATTCTG | 926 |
| FANCL | TTTCTCCGGACTTCGAGCCA | 927 | GCTGCCAAAAACTGACTATAAATGC | 928 |
| FANCL | GCCCCAGAACCGGTCGAAAA | 929 | GCGTGCTGTTGCACTCCGTG | 930 |
| FANCL | GAAGCGAGCCTGTTGCGCC | 931 | CTGTTGCACTCCGTGGAGGTTT | 932 |
| FANCL | GTTGCGCCAGTGCCCCCT | 933 | GCACTCCGTGGAGGTTTTTCTGG | 934 |
| FANCL | CTTCGAGCCATGGCGGTGAC | 935 | CGTGGAGGTTTTTCTGGCTCAAG | 936 |
| FANCL | CAGCGGACTGCGCATGTG | 937 | ATGCCTTTAGTGATTCTATTGCTGC | 938 |
| FANCL | TCTGCCCCAGAACCGGTC | 925 | CAGGAAAATCCACAAAATAATCTGG | 924 |
| FANCL | CAGTGCCCCCTGCTTCTG | 923 | ATCCACAAAATAATCTGGTGATTCTG | 926 |
| FANCL | CTGTGTTTCTCCGGACTTCG | 889 | CAGCTCTTGTCTATTCTTTAAGGCA | 944 |
| FANCL | AGGGATTCATCTCGGCTCAG | 911 | ATGGTACTGAAGCAGGTATCCG | 890 |
| FANCL | AGAACCGGTCGAAAACCGT | 913 | GCATCTTCTGCTTTTAACTTGATGG | 902 |
| FANCL | CGAAAACCGTGTATGAGGGATTCA | 919 | GAGGTGTCCAGGAGGCACAA | 918 |
| FANCL | GCCCCAGAACCGGTCGAAAA | 929 | GGAGGCACAAAATGGAACAGGA | 952 |
| FANCL | GTGACGGAAGCGAGCCTGTT | 901 | AAAATAATCTGGTGATTCTGCAGGATA | 954 |
| FANCL | CATGGCGGTGACGGAAGC | 917 | GCACAAAATGGAACAGGAAAATCC | 956 |
| FANCL | CATGGCGGTGACGGAAGC | 917 | GGGGAGGAGGAGGTAGTGCATA | 958 |
| FANCL | TTTCTCCGGACTTCGAGCCA | 927 | AATGGAACAGGAAAATCCACAAAA | 960 |
| FANCL | GCAGGACCCAGCAGGTCTAG | 915 | CAATAAGGCTTGAGTAGAACTGGG | 962 |
| FANCL | GAAGCGAGCCTGTTGCGCC | 931 | GGAGGAGGAGGTAGTGCATACAGCTCT | 964 |
| FANCL | CAGCGGACTGCGCATGTG | 937 | GGCTTGAGTAGAACTGGGGAGGAG | 966 |
| FANCL | GTGACGGAAGCGAGCCTGTT | 901 | GGAGGAGGAGGTAGTGCATACAGCTCT | 964 |
| FANCL | GGTCGAAAACCGTGTATGAGG | 969 | TCCCAACCAAGAGTTCCTATCTC | 970 |
| FANCL | AGAGCTTTTCTGTGTTTCTCCG | 895 | AGTTCCTATCTCTTCAATAAGGCTTG | 972 |
| FANCL | CCGTGTATGAGGGATTCATCTC | 891 | CAACCAAGAGTTCCTATCTCTTCAATA | 974 |
| FANCL | AGAACCGGTCGAAAACCGT | 913 | ATCTCTTCAATAAGGCTTGAGTAGAAC | 976 |
| FANCL | ACCCAGCAGGTCTAGAGCTTT | 899 | GGTAGTGCATACAGCTCTTGTCTATTC | 978 |
| FANCL | GCAGGTCTAGAGCTTTTCTGTGTT | 907 | GCAGGTATCCGCATACACAAG | 900 |
| FANCL | CAGTGCCCCCTGCTTCTG | 923 | TTTGCCTTCAACTTGAGAGTGA | 892 |
| FANCL | TTTCTCCGGACTTCGAGCCA | 927 | ACTGAAGCAGGTATCCGCATACA | 910 |
| FANCL | TCTGCCCCAGAACCGGTC | 925 | AGTGATTAAATGCTCTCTACCAGAAGC | 986 |
| FANCL | CGAAAACCGTGTATGAGGGATTCA | 919 | AAATGCTCTCTACCAGAAGCATCTTCT | 988 |
| FANCL | CATGTGCAGGACCCAGCAG | 989 | TTTAACTTGATGGTACTGAAGCAGGTA | 990 |
| FANCL | TCGAGCCATGGCGGTGAC | 991 | TGCCTTCAACTTGAGAGTGATTAAATG | 992 |
| FANCL | GCCCCAGAACCGGTCGAAAA | 929 | GAGGTGTCCAGGAGGCACAA | 918 |
| FANCL | GAAGCGAGCCTGTTGCGCC | 931 | GGAGGCACAAAATGGAACAGGA | 952 |
| FANCL | AGTTGCCTTAAAGAATAGACAAGAGC | 997 | TTTGCCTTCAACTTGAGAGTGA | 892 |
| FANCL | TGTATGAGGGATTCATCTCGG | 999 | AAATAATCTGGTGATTCTGCAGG | 914 |
| FANCL | GTGACGGAAGCGAGCCTGTT | 901 | ATCCACAAAATAATCTGGTGATTTCTG | 926 |
| FANCL | CTGTGTTTCTCCGGACTTCG | 889 | GAGGCACAAAATGGAACAGG | 912 |
| FANCL | GTGGATACCATCGAATAGTACAACAG | 1005 | GAGGCACAAAATGGAACAGG | 912 |
| FANCL | CATGGCGGTGACGGAAGC | 917 | GGCACAAAATGGAACAGGAAAATC | 920 |
| FANCL | TGGCAGCTGAGAACAATACTTAGTG | 1008 | GGCACAAAATGGAACAGGAAAATC | 920 |
| FANCL | GAAGCGAGCCTGTTGCGCC | 931 | TGTCCAGGAGGCACAAAATGGA | 922 |
| FANCL | ATGTAGTTGGCAGCTGAGAACA | 1011 | ATGGAACAGGAAAATCCACAAA | 1012 |
| FANCL | GAACAATACTTAGTGGATACCATCGA | 1013 | GCTGCCAAAAACTGACTATAAATGC | 928 |
| FANCL | GCTTTATGATGGAGTTGAAGATGC | 1015 | ATGCCTTTAGTGATTCTATTGCTGC | 938 |
| FANCL | TTGCCTGAAGATTTACAACTGAAG | 1017 | ATGCCTTTAGTGATTCTATTGCTGC | 938 |
| FANCL | CCTGATCTAATGAGCTTTATGATGG | 1018 | GATTCTATTGCTGCCAAAAACTG | 1019 |
| FANCL | GGATAGTGTTGCCTGAAGATTTACA | 1020 | TCTATTGCTGCCAAAAACTGAC | 1021 |
| FANCL | TCCACCTTAGGATAGTGTTGCC | 1022 | CCCAGAATGCCTTTAGTGATTC | 1023 |
| FANCL | CTAATGAGCTTTATGATGGAGTTGAA | 1024 | CCATAACATCCCAGAATGCC | 1025 |
| FANCL | GAATGCAGCACTCTCCTGATC | 1026 | TCGATTTCATCCATAACATCCC | 1027 |
| FANCL | CACCTTAGGATAGTGTTGCCTGAAGAT | 1028 | TAACATCCCAGAATGCCTTTAGTG | 1029 |
| FANCL | AGCACTCTCCTGATCTAATGAGC | 1030 | GGTCTTCTCATCGATTTCATCC | 1031 |
| FANCL | GAAGAGACTTCCACCTTAGGATAGTG | 1032 | TTTCATCCATAACATCCCAGAATG | 1033 |
| FANCL | ATTATTATGTAGTTGGCAGCTGAGAA | 1034 | TACAGCTCTTGTCTATTCTTTAAGGC | 1035 |
| FANCL | GTGGATACCATCCAATAGTACAACAG | 1005 | TTTGCCTTCAACTTGAGAGTGA | 892 |
| FANCL | TGGCAGCTGAGAACAATACTTAGTG | 1008 | ATGGTACTGAAGCAGGTATCCG | 890 |
| FANCL | GAACAATACTTAGTGGATACCATCGA | 1040 | TGCTCTCTACCAGAAGCATCTTC | 898 |
| FANCL | ATGTAGTTGGCAGCTGAGAACAATACT | 1042 | TTAACTTGATGGTACTGAAGCAGG | 896 |
| FANCL | TCCACCTTAGGATAGTGTTGCC | 1044 | CAACTTGAGAGTGATTAAATGCTCTC | 894 |
| FANCL | TTGCCTGAAGATTTACAACTGAAG | 1017 | GCAGGTATCCGCATACACAAG | 900 |
| FANCL | GGATAGTGTTGCCTGAAGATTTACA | 1020 | AGTGATTAAATGCTCTCTACCAGAAGC | 986 |
| FANCL | TGAAGATTTACAACTGAAGAATGCAAG | 1050 | ACTGAAGCAGGTATCCGCATACA | 910 |
| FANCL | GGTCGAAAACCGTGTATGAGG | 969 | CTACCAGAAGCATCTTCTGCTTT | 908 |
| FANCL | AGAACCGGTCGAAAACCGT | 913 | CAGGAAAATCCACAAAATAATCTGG | 924 |
| FANCL | CCTGCTTCTGCCCCAGAAC | 1056 | ATCCACAAAATAATCTGGTGATTCTG | 926 |
| FANCL | TCTGCCCCAGAACCGGTC | 925 | GGCACAAAATGGAACAGGAAAATC | 920 |
| FANCL | CAGTGCCCCCTGCTTCTG | 923 | ATGGAACAGGAAAATCCACAAA | 1012 |
| FANCL | GCCCCAGAACCGGTCGAAAA | 929 | TGTCCAGGAGGCACAAAATGGA | 922 |
| FANCL | CTGTGTTTCTCCGGACTTCG | 889 | TCCCAACCAAGAGTTCCTATCTC | 970 |
| FANCL | GCAGGTCTAGAGCTTTTCTGTGTT | 907 | ATCTCTTCAATAAGGCTTGAGTAGAAC | 976 |
| FANCL | GAGCCTGTTGCGCCAGTG | 905 | CAACCAAGAGTTCCTATCTCTTCAATA | 974 |
| FANCL | GCAGGACCCAGCAGGTCTAG | 1070 | ATCCACAAAATAATCTGGTGATTCTG | 926 |
| FANCL | TTTCTCCGGACTTCGAGCCA | 927 | TTTACCTGAGGTGTCCAGGAGG | 1073 |
| FANCL | GAGCCTGTTGCGCCAGTG | 905 | GCATCTTCTGCTTTAACTTGATGG | 902 |
| FANCL | GTGACGGAAGCGAGCCTGTT | 901 | TTCTGCTTTTAACTTGATGGTACTGAA | 906 |
| FANCL | AGAACCGGTCGAAAACCGT | 913 | CTACCAGAAGCATCTTCTGCTTT | 908 |
| FANCL | GCCCCAGAACCGGTCGAAAA | 929 | GGCACAAAATGGAACAGGAAAATC | 920 |
| FGFR1 | AGAACTGGGATGTGGAGCTG | 1082 | TGTTTCTTTCTCCTCTGAAGAGG | 1083 |
| FGFR1 | CTCTATGCTTGCGTAACCAGC | 1084 | TGTTTCTTTCTCCTCTGAAGAGG | 1083 |
| FGFR1 | ATGTGCAGAGCATCAACTGG | 1085 | TTTGGTGTTATCTGTTTCTTTCTCC | 1086 |
| FGFR1 | AGGTGGAGGTGCAGGACTC | 1087 | GGTTTGGTTTGGTGTTATCTGTTT | 1088 |
| FGFR1 | GGTGACCTGCTGCAGCTTC | 1089 | CATCATCATCATCATCCTCCG | 1090 |
| FGFR1 | AGCTGGCGGAAAGCAACC | 1091 | TCTGAAGAGGAGTCATCATCATCA | 1092 |
| FGFR1 | GGACGATGTGCAGAGCATC | 1093 | CCGAGGAGGGGAGAGCAT | 1094 |
| FGFR1 | AGAGCATCAACTGGCTGCG | 1095 | CATCATCATCCTCCGAGGAG | 1096 |
| FGFR1 | TGACACCACCTACTTCTCCGTC | 1097 | GAACTTCACTGTCTTGGCAGC | 1098 |
| FGFR1 | CCTACTTCTCCGTCAATGTTTCAG | 1099 | CACTGGAAGGGCATTTGAAC | 1100 |
| FGFR1 | ATCACAGGGGAGGAGGTGG | 1101 | GGATGTCCAATATGGAGCTACG | 1102 |
| FGFR1 | GGGCAGTGACACCACCTACT | 1103 | TCTTTTCCATCTTTTCTGGGG | 1104 |
| FGFR1 | TTGCGTAACCAGCAGCCC | 1105 | GCATTTGAACTTCACTGTCTTGG | 1106 |
| FGFR1 | CCGGCCTCTATGCTTGCGTA | 1107 | TCCATCTTTTCTGGGGATGTCC | 1108 |
| FGFR1 | AGGTGCAGGACTCCGTGC | 1109 | GCATGCAATTCTTTTCCATC | 1110 |
| FGFR1 | ACCGCACCCGCATCACAG | 1111 | CGGCACTGCATGCAATTCT | 1112 |
| FGFR1 | TAACCAGCAGCCCCTCGG | 1113 | TTTCAACCAGCGCAGTGTG | 1114 |
| FGFR1 | CCCTCGGGCAGTGACACCAC | 1115 | ACTGGAAGGGCATTTGAACTTCACTG | 1116 |
| FGFR1 | GAAAGCAACCGCACCCGCAT | 1117 | CTTTTCTGGGGATGTCCAATATGGAGC | 1118 |
| FGFR1 | CCCGCAGACTCCGGCCTCTA | 1119 | GGGTCCCACTGGAAGGGCAT | 1120 |
| FGFR1 | AGCAGCCCCTCGGGCAGT | 1121 | GCAGTGTGGGGTTTGGGGTC | 1122 |
| FGFR1 | ACCCGCATCACAGGGGAG | 1123 | TCTTGGCAGCCGGCACTG | 1124 |
| FGFR1 | CAGGACTCCGTGCCCGCAG | 1125 | GGGTTTGGGGTCCCACTGG | 1126 |
| FGFR1 | GGTGCAGCTGGCGGAAAG | 1127 | ACCAGCGCAGTGTGGGGTTT | 1128 |
| FGFR1 | CCGACCTTGCCTGAACAAG | 1129 | CATCATCATCATCATCCTCCG | 1090 |
| FGFR1 | AGAACTGGGATGTGGAGCTG | 1082 | CCGAGGAGGGGAGAGCAT | 1094 |
| FGFR1 | GTCACAGCCACACTCTGCAC | 1133 | CATCATCATCCTCCGAGGAG | 1096 |
| FGFR1 | ACACTCTGCACCGCTAGGC | 1135 | TCTGAAGAGGAGTCATCATCATCA | 1092 |
| FGFR1 | CTGTGCTGGTCACAGCCAC | 1137 | TGTTTCTTTCTCCTCTGAAGAGG | 1083 |
| FGFR1 | GAAGTGCCTCCTCTTCTGGG | 1139 | GGTTTGGTTTGGTGTTATCTGTTT | 1088 |
| FGFR1 | GCCTCCTCTTCTGGGCTGTG | 1141 | GCATACGGTTTGGTTTGGTG | 1142 |
| FGFR1 | TAGGCCGTCCCCGACCTTG | 1143 | TTTCTGGGGATGTCCAATATGG | 1144 |
| FGFR1 | CGTCCCCGACCTTGCCTGAA | 1145 | CTGGGGATGTCCAATATGGAGCTACG | 1146 |
| FGFR1 | GTCACAGCCACACTCTGCAC | 1133 | ACCAAGTCCAAATGGCAAGG | 1148 |
| FGFR1 | AGAACTGGGATGTGGAGCTG | 1082 | ACTTAGCCTCCTGGAGATCTGG | 1150 |
| FGFR1 | GAAGTGCCTCCTCTTCTGGG | 1139 | AAATGGCAAGGGAGTGATGG | 1152 |
| FGFR1 | ACACTCTGCACCGCTAGGC | 1135 | CCGAGTACCAAGTCCAAATGG | 1154 |
| FGFR1 | CCGACCTTGCCTGAACAAG | 1129 | CAGGGATACCACCACCTGTT | 1156 |
| FGFR1 | CTGTGCTGGTCACAGCCAC | 1137 | CACTAAGCCGAGTACCAAGTCC | 1158 |
| FGFR1 | GCCTCCTCTTCTGGGCTGTG | 1141 | CAAGGGAGTGATGGAGTGGAAG | 1160 |
| FGFR1 | CTAACTGCAGAACTGGGATGTG | 1161 | GAGCACCACTTAGCCTCCTG | 1162 |
| FGFR1 | ATGTGGAGCTGGAAGTGCCT | 1163 | GAGTGGAAGCTGGCCGAG | 1164 |
| FGFR1 | TCTTCTGGGCTGTGCTGGTC | 1165 | GTGATGGAGTGGAAGCTGGC | 1166 |
| FGFR1 | TTGTCACCAACCTCTAACTGCA | 1167 | TCCTGGAGATCTGGGCAAG | 1168 |
| FGFR1 | AGCTGGAAGTGCCTCCTCTT | 1169 | CTGGCCGAGCACCACTTAG | 1170 |
| FGFR1 | CGTCCCCGACCTTGCCTGAA | 1145 | CCACCACCTGTTCAGGGCCTCTA | 1172 |
| FGFR1 | TAGGCCGTCCCCGACCTTG | 1143 | CCTCTCCAGCAGAGCAGGGATA | 1174 |
| FGFR1 | ACAGCCACACTCTGCACCGCTAG | 1175 | GAGTACCAAGTCCAAATGGCAAGGGAG | 1176 |
| FGFR1 | GTGCTGGTCACAGCCACACTCTG | 1177 | CACCTGTTCAGGGCCTCTAATCACTA | 1178 |
| FGFR1 | CTGCACCGCTAGGCCGTCC | 1179 | CAGGGCCTCTAATCACTAAGCCGA | 1180 |
| FGFR1 | TCACCAACCTCTAACTGCAGAACTGG | 1181 | GGAAGCTGGCCGAGCACCAC | 1182 |
| FGFR1 | AGATGTGGAGCCTTGTCACC | 1183 | CTCTAATCACTAAGCCGAGTACCAA | 1184 |
| FGFR1 | AGATGTGGAGCCTTGTCACC | 1183 | TTTGGTGTTATCTGTTTCTTTCTCC | 1086 |
| FGFR1 | ATGTGGAGCTGGAAGTGCCT | 1163 | TCTGAAGAGGAGTCATCATCATCA | 1092 |
| FGFR1 | CTAACTGCAGAACTGGGATGTG | 1161 | CATCATCATCCTCCGAGGAG | 1096 |
| FGFR1 | GTCACAGCCACACTCTGCAC | 1133 | CACTGGAAGGGCATTTGAAC | 1100 |
| FGFR1 | CCGACCTTGCCTGAACAAG | 1129 | GGATGTCCAATATGGAGCTACG | 1102 |
| FGFR1 | ACACTCTGCACCGCTAGGC | 1135 | GAACTTCACTGTCTTGGCAGC | 1098 |
| FGFR1 | GCCTCCTCTTCTGGGCTGTG | 1141 | CGGCACTGCATGCAATTTCT | 1112 |
| FGFR1 | GTGCTGGTCACAGCCACACTCTG | 1177 | ACTGGAAGGGCATTTGAACTTCACTG | 1116 |
| FGFR1 | ACAGCCACACTCTGCACCGCTAG | 1175 | GGGTCCCACTGGAAGGGCAT | 1120 |
| FGFR1 | CTGCACCGCTAGGCCGTCC | 1179 | GCAGTGTGGGGTTTGGGGTC | 1204 |
| FGFR1 | GAGCCTTGTCACCAACCTCT | 1205 | TCTTTTCCATCTTTTCTGGGG | 1104 |
| FGFR1 | TCACCAACCTCTAACTGCAGAACTGG | 1181 | TCCATCTTTTCTGGGGATGTCC | 1108 |
| FGFR1 | CGTCCCCGACCTTGCCTGAA | 1145 | TCTTGGCAGCCGGCACTG | 1124 |
| FGFR1 | TAGGCCGTCCCCGACCTTG | 1143 | ACCAGCGCAGTGTGGGGTTT | 1128 |
| FGFR1 | CATGGAGATGTGGAGCCTTG | 1213 | TTTCTGGGGATGTCCAATATGG | 1144 |
| FGFR1 | AGTATCCATGGAGATGTGGAGC | 1215 | GCATTTGAACTTCACTGTCTTGG | 1106 |
| FGFR1 | AGGATCGAGCTCACTGTGGA | 1217 | CTGCATGCAATTTCTTTTCCA | 1218 |
| FGFR1 | GTGGAGCCTTGTCACCAACCTCTAACT | 1219 | TTGCCATTTTTCAACCAGCG | 1220 |
| FGFR1 | TCGAGCTCACTGTGGAGTATCC | 1221 | GCATACGGTTTGGTTTGGTG | 1142 |
| FGFR1 | CCGACCTTGCCTGAACAAG | 1129 | TTTGGTGTTATCTGTTTCTTTCTCC | 1086 |
| FGFR1 | CTGTGCTGGTCACAGCCAC | 1137 | TCTGAAGAGGAGTCATCATCATCA | 1092 |
| FGFR1 | CTAACTGCAGAACTGGGATGTG | 1161 | GGATGTCCAATATGGAGCTACG | 1102 |
| FGFR1 | AGATGTGGAGCCTTGTCACC | 1183 | GCATACGGTTTGGTTTGGTG | 1142 |
| FGFR1 | AGAACTGGGATGTGGAGCTG | 1082 | TGCTGGTTACGCAAGCATAG | 1232 |
| FGFR1 | TTGTCACCAACCTCTAACTGCA | 1167 | TTGATGCTCTGCACATCGTC | 1234 |
| FGFR1 | GTCACAGCCACACTCTGCAC | 1133 | ACATTGACGGAGAAGTAGGTGG | 1236 |
| FGFR1 | ATGTGGAGCTGGAAGTGCCT | 1163 | AAGCATAGAGGCCGGAGTCT | 1238 |
| FGFR1 | AGATGTGGAGCCTTGTCACC | 1183 | GCAGCCAGTTGATGCTCTG | 1240 |
| FGFR1 | GAGCCTTGTCACCAACCTCT | 1205 | AGTCCTGCACCTCCACCTC | 1242 |
| FGFR1 | CCGACCTTGCCTGAACAAG | 1129 | AGAAGTAGGTGGTGTCACTGCC | 1244 |
| FGFR1 | AGGATCGAGCTCACTGTGGA | 1217 | GACCAGGAAGGACTCCACTTC | 1246 |
| FGFR1 | CTAACTGCAGAACTGGGATGTG | 1161 | TACGCAAGCATAGAGGCCG | 1248 |
| FGFR1 | AGTATCCATGGAGATGTGGAGC | 1215 | GTTGCTTTCCGCCAGCTG | 1250 |
| FGFR1 | GAAGTGCCTCCTCTTCTGGG | 1139 | TCCACCTCCTCCCCTGTGAT | 1252 |
| FGFR1 | TCTTCTGGGCTGTGCTGGTC | 1165 | CGAGGGGCTGCTGGTTAC | 1254 |
| FGFR1 | TCGAGCTCACTGTGGAGTATCC | 1221 | AAGCTGCAGCAGGTCACC | 1256 |
| FGFR1 | ACACTCTGCACCGCTAGGC | 1135 | GCACCTCCACCTCCTCCC | 1258 |
| FGFR1 | AGCTGGAAGTGCCTCCTCTT | 1169 | ACAGCGAAGCTGCAGCAG | 1260 |
| FGFR1 | GCCTCCTCTTCTGGGCTGTG | 1141 | CGGGTGCGGTTGCTTTCC | 1262 |
| FGFR1 | TCACCAACCTCTAACTGCACAACTGG | 1181 | GGAAGGACTCCACTTCCACAGG | 1264 |
| FGFR1 | ACAGCCACACTCTGCACCGCTAG | 1175 | TCTGCACATCGTCCCGCAG | 1266 |
| FGFR1 | CTGTGCTGGTCACAGCCAC | 1137 | TGGTGTCACTGCCCGAGG | 1268 |
| FGFR1 | GTGCTGGTCACAGCCACACTCTG | 1177 | AGCCAGTTGATGCTCTGCACATC | 1270 |
| FGFR1 | GTGGAGCCTTGTCACCAACCTCTAACT | 1219 | CGTCCCGCAGCCAGTTGAT | 1272 |
| FGFR1 | CGTCCCCGACCTTGCCTGAA | 1145 | TCCTCCCCTGTGATGCGGGT | 1274 |
| FGFR1 | CTGCACCGCTAGGCCGTCC | 1179 | GAGTCTGCGGGCACGGAGTC | 1276 |
| FGFR1 | CAGTTTGAAAAGGAGGATCGAG | 1277 | GGGTGGACCAGGAAGGACTC | 1278 |
| FGFR1 | TAGGCCGTCCCCGACCTTG | 1143 | CTTTCCGCCAGCTGCACCC | 1280 |
| FGFR1 | AAAAGGAGGATCGAGCTCACTG | 1281 | CAGCCGACAGCGAAGCTG | 1282 |
| FGFR1 | CTGTGGAGTATCCATGGAGATG | 1283 | CACGGAGTCCTGCACCTC | 1284 |
| FGFR1 | CCATGGAGATGTGGAGCCTTGTC | 1285 | ATCGTCCCGCAGCCGACAG | 1286 |
| FGFR1 | AGGAGGATCGAGCTCACTGTGGAGTAT | 1287 | AGGTCACCGGGGTGGACCAG | 1288 |
| FGFR1 | GAGCTCACTGTGGAGTATCCATGGAGA | 1289 | CCAGCTGCACCCCGTCCC | 1290 |
| FGFR1 | CCAGGACCCGAACAGAGC | 1291 | CTCCACTTCCACAGGGGCTC | 1292 |
| FGFR1 | AGGCGGAACCTCCACGCC | 1293 | TGCAGCAGGTCACCGGGGT | 1294 |
| FGFR1 | ACACGCCCGCTCGCACAA | 1295 | CTTCCACAGGGGCTCCCCAG | 1296 |
| FGFR1 | GGACTCTCCCGAGGCGGAAC | 1297 | AGGGGCTCCCCAGGGCTG | 1298 |
| FGFR1 | AACCTCCACGCCGAGCGAG | 1299 | TAGAGGCCGGAGTCTGCGGG | 1300 |
| FGFR1 | CTGCACCGCTAGGCCGTCC | 1179 | CCATCTTTTCTGGGGATGTCCAA | 1302 |
| FGFR2 | TGCAGATGGGATTAACGTCC | 1303 | GTGTCATCCTCATCATCTCCG | 1304 |
| FGFR2 | TTTCATCTGCCTGGTCGTG | 1305 | GTGTCATCCTCATCATCTCCG | 1304 |
| FGFR2 | TCACCATGGCAACCTTGTC | 1307 | ACAAAATCTTCCGCACCATC | 1308 |
| FGFR2 | GGCCCTCCTTCAGTTTAGTTG | 1309 | TCTTGTTGTTACTGTTCTCACTGACA | 1310 |
| FGFR2 | TGCAGATGGGATTAACGTCC | 1303 | ATCTCCGGATGAGATGGCAT | 1312 |
| FGFR2 | GGGTCGTTTCATCTGCCTG | 1313 | ACCATCGGTGTCATCCTCAT | 1314 |
| FGFR2 | GATTGGTACCGTAACCATGGTC | 1315 | CTCATCATCTCCGGATGAGATG | 1316 |
| FGFR2 | TGGTCGTGGTCACCATGG | 1317 | CTCACTGACAAAATCTTCCGC | 1318 |
| FGFR2 | ATCTGCCTGGTCGTGGTCAC | 1319 | ATCTTCCGCACCATCGGTGT | 1320 |
| FGFR2 | ATGGCAACCTTGTCCCTGG | 1321 | TTACTGTTCTCACTGACAAAATCTTCC | 1322 |
| FGFR2 | TGAGGATACCACATTAGAGCCAG | 1323 | TTTCTGTGTTGGTCCAGTATGG | 1324 |
| FGFR2 | CCTTCAGTGTAGTTGAGGATACCAC | 1325 | GTTGGTCCAGTATGGTGCTC | 1326 |
| FGFR2 | GTTTAGTTGAGGATACCACATTAGAGC | 1327 | CCGCTTTTCCATCTTTTCTG | 1328 |
| FGFR2 | TCGTGGTCACCATGGCAACC | 1329 | TCCATCTTTTCTGTGTTGGTCCAGTAT | 1330 |
| FGFR2 | ACCTTGTCCCTGGCCCGG | 1331 | CATGGAGCCGCTTTTCCATC | 1332 |
| FGFR4 | GAAGCACATCGTCATCAACG | 1333 | AAGTGGGAGACTTGGTTCTGC | 1334 |
| FGFR4 | ATCAATAGCTCAGAGGTGGAGG | 1335 | AAGTGGGAGACTTGGTTCTGC | 1334 |
| FGFR4 | CTGTACCTGCGGAACGTGTC | 1336 | GAGAACTGCAAAGTGGGAGACT | 1337 |
| FGFR4 | GAGGTGGAGGTCCTGTACCTG | 1338 | AGGCTGTCACATGTGAGGTG | 1339 |
| FGFR4 | AATTCCATCGGCCTCTCCTA | 1340 | TCCAGGGAGAACTGCAAAGT | 1341 |
| FGFR4 | AGGCGAGTACACCTGCCTC | 1342 | AGACTTGGTTCTGCCTGCTG | 1343 |
| FGFR4 | ACTGCAGACATCAATAGCTCAGAG | 1344 | TGGAGTCAGGCTGTCACATG | 1345 |
| FGFR4 | GCTCAGAGGTGGAGGTCCTG | 1346 | ACTGCAAAGTGGGAGACTTGGTTC | 1347 |
| FGFR4 | AGGTCCTGTACCTGCGGAAC | 1348 | ACATGTGAGGTGGGGGATG | 1349 |
| FGFR4 | GAACGTGTCAGCCGAGGAC | 1350 | GTTCTGCCTGCTGGAGTCAG | 1351 |
| FGFR4 | AATAGCTCAGAGGTGGAGGTCCTGTAC | 1352 | CCTGCTGGAGTCAGGCTGTC | 1353 |
| FGFR4 | CTGCGGAACGTGTCAGCCG | 1354 | GTGGGGGATGCGCCCAGTAC | 1355 |
| GATA3 | CTTCGGATGCAAGTCCAGG | 1356 | AAGTCCTCCAGTGAGTCATGC | 1357 |
| GATA3 | CTTCGGATGCAAGTCCAGG | 1356 | TTGTGAAGCTTGTAGTAGAGCCC | 1358 |
| GATA3 | AGCATGAAGCTGGAGTCGTC | 1359 | TGTGGTGGTCTGACAGTTCG | 1360 |
| GATA3 | CTACGTGCCCGAGTACAGCT | 1361 | TCCAGAGTGTGGTTGTGGTG | 1362 |
| GATA3 | ATCACCACCTACCCGCCCTA | 1363 | ATTGGCATTCCTCCTCCAGA | 1364 |
| GATA3 | AGTACAGCTCCGGACTCTTCC | 1365 | GTGTGGTTGTGGTGGTCTGA | 1366 |
| GATA3 | ACCACCCCATCACCACCTAC | 1367 | TAGTAGAGCCCACAGGCATTG | 1368 |
| GATA3 | AAGCTGGAGTCGTCCCACTC | 1369 | TCTGACAGTTCGCACAGGAC | 1370 |
| GATA3 | CTCCTCGTCGACCCACCAC | 1371 | CACAGGCATTGCAGACAGG | 1372 |
| GATA3 | CTCCCGTGGCAGCATGAC | 1373 | ATTCCTCCTCCAGAGTGTGGTT | 1374 |
| GATA3 | AAAGAGTGCCTCAAGTACCAGG | 1375 | TGCTCTCCTGGCTGCAGA | 1376 |
| GATA3 | ACCTACCCGCCCTACGTGC | 1377 | GACGTCCCTGCTCTCCTGG | 1378 |
| GATA3 | CCGACAGCATGAAGCTGGAG | 1379 | AGTTCGCACAGGACGTCCCT | 1380 |
| GATA3 | GATGCAAGTCCAGGCCCAAG | 1381 | AGCTTGTAGTAGAGCCCACAGGC | 1382 |
| GATA3 | GACCCACCACCCCATCAC | 1383 | GTCCCCATTGGCATTCCTC | 1384 |
| GATA3 | ACCGGCTTCGGATGCAAGTC | 1385 | TGCAGACAGGGTCCCCATTG | 1386 |
| GATA3 | GCCCGAGTACAGCTCCGGAC | 1387 | AGTGTGGTTGTGGTGGTCTGACAGTTC | 1388 |
| GATA3 | TGGAGCCTCCTCGTCGAC | 1389 | CACAGGACGTCCCTGCTCTC | 1390 |
| GATA3 | CCGCCCTACGTGCCCGAGTA | 1391 | GGCATTGCAGACAGGGTCCC | 1392 |
| GATA3 | AGCATGACCGCCCTGGGT | 1393 | ACAGGGTCCCCATTGGCATT | 1394 |
| GATA3 | AAGGCCCGGTCCAGCACAG | 1395 | GGCCGGGTTAAACGAGCTGTT | 1396 |
| GATA3 | CCTGGGTGGAGCCTCCTC | 1397 | TGCCTTCCTTCTTTCATAGTCAGG | 1398 |
| GATA3 | CCCCCACCGGCTTCGGAT | 1399 | CGGGTTAAACGAGCTGTTCTTGGG | 1400 |
| GATA3 | AGTCGTCCCACTCCCGTGG | 1401 | TCCTTCTTCATAGTCAGGGGTCTG | 1402 |
| GATA3 | AAAGAGTGCCTCAAGTACCAGG | 1375 | CTTCGCTTGGGCTTAATGAG | 1404 |
| GATA3 | AGCATGAAGCTGGAGTCGTC | 1359 | AGGCGTTGCACAGGTAGTGT | 1406 |
| GATA3 | ATCACCACCTACCCGCCCTA | 1363 | CACAGTTCACACACTCCCTGC | 1408 |
| GATA3 | CTTCGGATGCAAGTCCAGG | 1356 | CCGGTTCTGTCCGTTCATTT | 1410 |
| GATA3 | AGTACAGCTCCGGACTCTTCC | 1365 | CCGTTCATTTTGTGATAGAGGC | 1412 |
| GATA3 | ACCACCCCATCACCACCTAC | 1367 | TAGTGTCCCGTGCCATCTC | 1414 |
| GATA3 | AAGCTGGAGTCGTCCCACTC | 1369 | TTGCACAGGTAGTGTCCCGT | 1416 |
| GATA3 | CTACGTGCCCGAGTACAGCT | 1361 | GAGGTTGCCCCACAGTTCAC | 1418 |
| GATA3 | CTCCTCGTCGACCCACCAC | 1371 | TGCCCCACAGTTCACACACT | 1420 |
| GATA3 | CCGACAGCATGAAGCTGGAG | 1379 | CTTAATGAGGGGCCGGTTCT | 1422 |
| GATA3 | ACCTACCCGCCCTACGTGC | 1377 | CATCTCGCCGCCACAGTG | 1424 |
| GATA3 | TGGAGCCTCCTCGTCGAC | 1389 | TTTGTGATAGAGCCCGCAG | 1426 |
| GATA3 | GACCCACCACCCCATCAC | 1383 | GTTCTGTCCGTTCATTTTGTGAT | 1428 |
| GATA3 | CTCCCGTGGCAGCATGAC | 1373 | ACAGTGGGGTCGAGGTTGC | 1430 |
| GATA3 | ACCGGCTTCGGATGCAAGTC | 1385 | CTTGGGCTTAATGAGGGGCC | 1432 |
| GATA3 | GCCCGAGTACAGCTCCGGAC | 1387 | GGGTCGAGGTTGCCCCACAG | 1434 |
| GATA3 | AGTCGTCCCACTCCCGTGG | 1401 | CACAGGTAGTGTCCCGTGCCATC | 1436 |
| GATA3 | GATGCAAGTCCAGGCCCAAG | 1381 | GATAGAGCCCGCAGGCGTTG | 1438 |
| GATA3 | CCGCCCTACGTGCCCGAGTA | 1391 | AGGGGCCGGTTCTGTCCGTT | 1440 |
| GATA3 | AGCATGACCGCCCTGGGT | 1393 | CCCGCAGGCGTTGCACAG | 1442 |
| GATA3 | CCCGGCAGGACGAGAAAGAG | 1443 | CCGCCACAGTGGGGTCGAG | 1444 |
| GATA3 | CCTGGGTGGAGCCTCCTC | 1397 | TCCAGAGTGTGGTTGTGGTG | 1362 |
| GATA3 | AAGGCCCGGTCCAGCACAG | 1395 | ACAGGGTCCCCATTGGCATT | 1394 |
| GATA3 | GACGAGAAAGAGTGCCTCAAGT | 1449 | TGCTCTCCTGGCTGCAGA | 1376 |
| GATA3 | CCTCAAGTACCAGGTGCCC | 1451 | CACAGGACGTCCCTGCTCTC | 1390 |
| GATA3 | CCCCCACCGGCTTCGGAT | 1399 | GAGCCCACAGGCATTGCAGA | 1454 |
| GATA3 | CCTGCCCGACAGCATGAAG | 1455 | CTGACAGTTCGCACAGGACG | 1456 |
| GATA3 | GTGGCAGCATGACCGCCCT | 1457 | GACGTCCCTGCTCTCCTGGC | 1458 |
| GATA3 | GACCGCCCTGGGTGGAGC | 1459 | CCCCATTGGCATTCCTCCTC | 1460 |
| GATA3 | GTGCCCCTGCCCGACAGC | 1461 | CAGTTCGCACAGGACGTCCCTG | 1462 |
| GATA3 | GGACCCATCGCTGTCCAC | 1463 | TGTGGTGGTCTGACAGTTCG | 1360 |
| GATA3 | TGTCCACCCCAGGCTCGG | 1465 | GGTGGTCTGACAGTTCGCACAGG | 1466 |
| GATA3 | ATCGCTGTCCACCCCAGG | 1467 | GTGTGGTTGTGGTGGTCTGA | 1366 |
| GNB3 | AGGTCCAGCCAGAGCCCAA | 1469 | ACTCGTCCCACCACCTCTAG | 1470 |
| GNB3 | AGAGTGACCCCTCGACCTGT | 1471 | TGCCAGAGTAACGTCAGCAC | 1472 |
| GNB3 | GAGCCAGAGTGACCCCTCG | 1473 | AGAGTAACGTCAGCACAGGCTT | 1474 |
| GNB3 | CCCTCGACCTGTCAGCCATG | 1475 | TAACGTCAGCACAGGCTTTCCTGG | 1476 |
| GNB3 | AGATGGAGCAACTGCGTCAG | 1477 | ACTCGTCCCACCACCTCTAG | 1470 |
| GNB3 | CAGCTCAAGAAGCAGATTGC | 1479 | GTGCATGGCGTAAATCTTGG | 1480 |
| GNB3 | TCAGGAAGCGGAGCAGCT | 1481 | TAAATCTTGGCCAGGTGTCC | 1482 |
| GNB3 | CAACTGCGTCAGGAAGCG | 1483 | CAGGTGTCCCCTTAACGTCC | 1484 |
| GNB3 | ATGGGGGAGATGGAGCAACT | 1485 | GTCCGCATCTGGACTCGTC | 1486 |
| GNB3 | AAGCGGAGCAGCTCAAGAAG | 1487 | TAGAATCAGTGGCCCAGTGC | 1488 |
| GNB3 | TCAGCCATGGGGGAGATG | 1489 | ACCACCTCTAGGCCAGACACC | 1490 |
| GNB3 | GCGGAGCAGCTCAAGAAGCAGATT | 1491 | GGCCCAGTGCATGGCGTAAAT | 1492 |
| GNB3 | CTGCGTCAGGAAGCGGAGCA | 1493 | TCTTGGCCAGGTGTCCCCTTAA | 1494 |
| GNB3 | ACCTGTCAGCCATGGGGGAG | 1495 | GCATCTGGACTCGTCCCACC | 1496 |
| GNB3 | AGGTCCAGCCAGAGCCCAA | 1469 | ATCTGGACTCGTCCCACCACCTCT | 1498 |
| GNB3 | ACCGGAGCTGGAAACCCG | 1499 | CTTAACGTCCGCCGCGTCC | 1500 |
| GNB3 | CAGGAACCGGAGCTGGAAAC | 1501 | TGGCGTAAATCTTGGCCAGG | 1502 |
| HMGA1 | CCCAGCCATCACTCTTCC | 1503 | GAGATGCCCTCCTCTTCCTC | 1504 |
| HMGA1 | CCCAGCCATCACTCTTCC | 1503 | TTTGCTTCCCTTTGGTCG | 1505 |
| HMGA1 | AAGGGAAGATGAGTGAGTCGAG | 1506 | CTCTTAGGTGTTGGCACTTCG | 1507 |
| HMGA1 | TCTTCCACCTGCTCCTTAGAGA | 1508 | ACCCTTGTTTTTGCTTCCCT | 1509 |
| HMGA1 | CCATCACTCTTCCACCTGCT | 1510 | CCCGAGGTCTCTTAGGTGTTG | 1511 |
| HMGA1 | AGTGAGTCGAGCTCGAAGTCC | 1512 | CTTGTTTTTGCTTCCCTTTGGTC | 1513 |
| HMGA1 | AAGATGAGTGAGTCGAGCTCGAA | 1514 | GTGTTGGCACTTCGCTGGG | 1515 |
| HMGA1 | CACCTGCTCCTTAGAGAAGGGAA | 1516 | GTCGGCCCCGAGGTCTCTTA | 1517 |
| HMGA1 | CTCGAAGTCCAGCCAGCCCTT | 1518 | CCGAGGTCTCTTAGGTGTTGGCACTTC | 1519 |
| HMGA1 | ATCCCAGCCATCACTCTTCCACCT | 1520 | CTTTGGTCGGCCCCGAGGT | 1521 |
| HMGA1 | TGGCCTCCAAGCAGGAAA | 1522 | GAGATGCCCTCCTCTTCCTC | 1504 |
| HMGA1 | TCCTTAGAGAAGGGAAGATGAGTG | 1524 | TGTCCAGTCCCAGAAGGAAG | 1525 |
| HMGA1 | AAAGGACGGCACTGAGAAGC | 1526 | GAGGACTCCTGCGAGATGC | 1527 |
| HMGA1 | TCCAAGCAGGAAAAGGACG | 1528 | GAGCGGAGCAAAGCTGTC | 1529 |
| HMGA1 | CAGCCCTTGGCCTCCAAG | 1530 | ATGGGTCACTGCTCCTCCTC | 1531 |
| HMGA1 | CAGGAAAAGGACGGCACTGA | 1532 | GTCCCAGAAGGAAGCTGCTC | 1533 |
| HMGA1 | TCCAGCCAGCCCTTGGCCT | 1534 | TCCTGCGAGATGCCCTCCTC | 1535 |
| HMGA1 | GGCACTGAGAAGCGGGGC | 1536 | AGTGAGGAGCAGGCGGCAC | 1537 |
| HMGA1 | CTGGCGCGGCTCCAAGAAG | 1538 | CCTCCGAGGACTCCTGCGAG | 1539 |
| HMGA1 | TCTAATTGGGACTCCGAGCC | 1540 | TCTTGGCAGCACCCTTGTTT | 1541 |
| HMGA1 | GCTATTTCTGGCGCTGGC | 1542 | GCAGCACCCTTGTTTTTGCT | 1543 |
| HMGA1 | CCGGGGCTATTTCTGGCGCT | 1544 | CCGGGTCTTGGCAGCACC | 1545 |
| HMGA1 | GTCCTCAGCGCCCAGCAC | 1546 | TCACTGCTCCTCCTCCGAGG | 1547 |
| HMGA1 | ATCCGCATTTGCTACCAGC | 1548 | CTCCTCCTCCGAGGACTCCT | 1549 |
| HMGA1 | AGCCAGGCCGGTCCTCAG | 1550 | CACGCATGGGTCACTGCTC | 1551 |
| HMGA1 | CATTTGCTACCAGCGGCGG | 1552 | GAGCAGGCGGCACGCATG | 1553 |
| HMGA1 | GCTCCTCTAATTGGGACTCC | 1554 | TCCTGGAGTTGTGGTGGTTT | 1555 |
| HMGA1 | ACTCCGAGCCGGGGCTATTT | 1556 | TCTGCCCCTTGGTTTCCTTC | 1557 |
| HMGA1 | TTTTAAGCTCCCCTGAGCC | 1558 | TTTCCTTCCTGGAGTTGTGG | 1559 |
| HMGA1 | CTCCCCTGAGCCGGTGCTG | 1560 | GTTTCCTTCCTGGAGTTGTGGTGGTTT | 1561 |
| HMGA1 | GTGCTGCGCTCCTCTAATTG | 1562 | CCTTGGTTTCCTTCCTGGAG | 1563 |
| HMGA1 | AGCCGGTGCTGCGCTCCT | 1564 | TTTGGGTCTGCCCCTTGGTT | 1565 |
| HMGA1 | TGGGTCGCTCTTTTTAAGCTC | 1566 | TCCTCCAGTGAGGAGCAGG | 1567 |
| HMGA1 | GCTCTTTTTAAGCTCCCCTG | 1568 | AGCTGCTCCTCCAGTGAGG | 1569 |
| HMGA1 | GCCCCTGGGTCGCTCTTTT | 1570 | CAGAAGGAAGCTGCTCCTCCAG | 1571 |
| HMGA1 | TCCAGCCAGCCCTTGGCCT | 1534 | CTTCCCTTTGGTCGGCCCC | 1573 |
| HMGA1 | TCGAGCTCGAAGTCCAGCCA | 1574 | CACGCATGGGTCACTGCTC | 1551 |
| HMGA1 | AGCCCTTGGCCTCCAAGCAG | 1576 | TCCTGCGAGATGCCCTCCTC | 1535 |
| HMGA1 | CTGGCGCGGCTCCAAGAAG | 1538 | TGCTCCTCCTCCGAGGACTC | 1579 |
| HMGA1 | GTGCTGCGCTCCTCTAATTG | 1562 | GCAGCACCCTTGTTTTTGCT | 1543 |
| HMGA1 | AGCCAGGCCGGTCCTCAG | 1550 | GGTCACTGCTCCTCCTCCG | 1583 |
| HMGA1 | ACTCCGAGCCGGGGCTATTT | 1556 | CCGGGTCTTGGCAGCACC | 1585 |
| HMGA1 | GTCCTCAGCGCCCAGCAC | 1546 | CAGAAGGAAGCTGCTCCTCCAG | 1571 |
| HMGA1 | GCCGGGGCTATTTCTGGC | 1588 | TCCTCCAGTGAGGAGCAGG | 1567 |
| HMGA1 | TGGGTCGCTCTTTTTAAGCTC | 1566 | CCTTGGTTTCCTTCCTGGAG | 1563 |
| HMGA1 | GCCCCTGGGTCGCTCTTTT | 1570 | TCTGCCCCTTGGTTTCCTTC | 1557 |
| HMGA1 | GCTCTTTTTAAGCTCCCCTG | 1568 | AAGCTGCTCCTCCAGTGAGG | 1595 |
| HMGA1 | CCATCACTCTTCCACCTGCT | 1510 | ATGGGTCACTGCTCCTCCTC | 1531 |
| HMGA1 | TCTTCCACCTGCTCCTTAGAGA | 1508 | AAGCTGCTCCTCCAGTGAGG | 1595 |
| HMGA1 | AAGGGAAGATGAGTGAGTCGAG | 1506 | TCCTCCAGTGAGGAGCAGG | 1567 |
| HMGA1 | CCCAGCCATCACTCTTCC | 1503 | CTCCTCCTCCGAGGACTCCT | 1549 |
| HMGA1 | ATCCCAGCCATCACTCTTCCACCT | 1520 | TCACTGCTCCTCCTCCGAGG | 1547 |
| HMGA1 | CACCTGCTCCTTAGAGAAGGGAA | 1516 | CAGAAGGAAGCTGCTCCTCCAG | 1571 |
| HMGA1 | AGTGAGTCGAGCTCGAAGTCC | 1512 | CACGCATGGGTCACTGCTC | 1551 |
| HMGA1 | AGCCCTTGGCCTCCAAGCAG | 1576 | GAGCAGGCGGCACGCATG | 1553 |
| HMGA1 | AAGATGAGTGAGTCGAGCTCGAA | 1514 | AGCAAAGCTGTCCAGTCCC | 1613 |
| HMGA1 | AGCGCTGGTAGGGAGTCAG | 1614 | CTGGTGTGCTGTGTAGTGTGG | 1615 |
| HMGA1 | AAGCAGCCTCCGGTGAGTC | 1616 | TGTGTAGTGTGGTGGTGAGGG | 1617 |
| HMGA1 | TCGAAGTCCAGCCAGCCCTT | 1618 | CAAAGCTGTCCAGTCCCAGAAGG | 1619 |
| HMGA1 | CTCCGGTGAGTCCCGGGA | 1620 | GTGTGCTGTGTAGTGTGGTGGTGA | 1621 |
| HMGA1 | GGGACAGCGCTGGTAGGGAG | 1622 | GTGAGGGCACAGGTGGAAGAT | 1623 |
| HMGA1 | CGAAGTGCCAACACCTAAGAG | 1624 | TGTCCAGTCCCAGAAGGAAG | 1525 |
| HMGA1 | CCAACACCTAAGAGACCTCGG | 1626 | AAGCTGCTCCTCCAGTGAGG | 1595 |
| HMGA1 | CGACCAAAGGGAAGCAAA | 1268 | GAGCGGAGCAAAGCTGTC | 1629 |
| HMGA1 | AAGGGAAGCAAAAACAAGGG | 1630 | AGCAAAGCTGTCCAGTCCC | 1613 |
| HMGA1 | CCCAGCGAAGTGCCAACAC | 1632 | CAAAGCTGTCCAGTCCCAGAAGG | 1619 |
| HMGA1 | CCTAAGAGACCTCGGGGCCG | 1634 | AGTGAGGAGCAGGCGGCAC | 1537 |
| HMGA1 | GGGGCCGACCAAAGGGAAG | 1636 | CAGAAGGAAGCTGCTCCTCCAG | 1571 |
| HMGA1 | TCGAAGTCCAGCCAGCCCTT | 1618 | CCTCCGAGGACTCCTGCGAG | 1539 |
| HMGA1 | AGCCAGGCCGGTCCTCAG | 1550 | GGTGGGAGCGGAGCAAAGCT | 1641 |
| HMGA1 | GTCCTCAGCGCCCAGCAC | 1546 | ATGGTGGGCCTGGGGAAG | 1643 |
| HMGA1 | TTTGCTACCAGCGGCGGC | 1644 | CTGGGGAAGGGGTGGGGG | 1645 |
| HMGA1 | CGAGCTCGAAGTCCAGCCAG | 1646 | GGTGGGAGCGGAGCAAAGCT | 1641 |
| HMGA1 | AGCCAGGCCGGTCCTCAG | 1550 | CTGGGGAAGGGGTGGGGG | 1645 |
| HMGA1 | AGCCCTTGGCCTCCAAGCAG | 1576 | CCGAGGTCTCTTAGGTGTTGGCACTTC | 1519 |
| HMGA1 | TGGCCTCCAAGCAGGAAAAG | 1652 | AAGCTGCTCCTCCAGTGAGG | 1595 |
| HMGA1 | AGCCAGGCCGGTCCTCAG | 1550 | CTGCGAGATGCCCTCCTCTT | 1655 |
| HMGA1 | CTGGCGCGGCTCCAAGAAG | 1538 | TTTGGGTCTGCCCCTTGGTT | 1565 |
| HMGA1 | CTCCCCTGAGCCGGTGCTG | 1560 | GGTCACTGCTCCTCCTCCGA | 1659 |
| HMGA1 | AGCCGGTGCTGCGCTCCT | 1564 | TCACTGCTCCTCCTCCGAGGACTC | 1661 |
| HMGA1 | CGGTGCTGCGCTCCTCTAATT | 1662 | CGGCACGCATGGGTCACT | 1663 |
| HMGA1 | TGCTGCGCTCCTCTAATTGGGACT | 1664 | GTTTCCTTCCTGGAGTTGTGGTGGTTT | 1561 |
| HMGA1 | GCTCTTTTTAAGCTCCCCTG | 1568 | CCTTGGTTTCCTTCCTGGAG | 1563 |
| HMGA1 | GGTTTCAGATCCGCATTTGC | 1668 | CAGAAGGAAGCTGCTCCTCCAG | 1571 |
| HMGA1 | TCTCTCCCGGTTTCAGATCC | 1670 | AGCAAAGCTGTCCAGTCCC | 1613 |
| HMGA1 | TTTCAGATCCGCATTTGCTACCAG | 1672 | CAAAGCTGTCCAGTCCCAGAAGG | 1619 |
| HMGA1 | TCCTTAGAGAAGGGAAGATGAGTG | 1524 | CACTTCGCTGGGCTCCTT | 1675 |
| HMGA1 | CGAGCTCGAAGTCCAGCCAG | 1646 | GGTCACTGCTCCTCCTCCGA | 1659 |
| HMGA1 | CTGGCGCGGCTCCAAGAAG | 1538 | CCTGCGAGATGCCCTCCTCTT | 1679 |
| HMGA1 | GCCGGGGCTATTTCTGGC | 1588 | AGATGCCCTCCTCTTCCTCC | 1681 |
| HMGA1 | TTTGCTACCAGCGGCGGC | 1644 | GAGCAGGCGGCACGCATG | 1553 |
| HMGA1 | GCTCCTCTAATTGGGACTCC | 1554 | GCAGCACCCTTGTTTTTGCT | 1543 |
| HMGA1 | TGGGTCGCTCTTTTTAAGCTC | 1566 | TCCTGGAGTTGTGGTGGTTT | 1555 |
| HMGA1 | ATCCGCATTTGCTACCAGC | 1548 | CCTTGGTTTCCTTCCTGGAG | 1563 |
| HMGA1 | GTGCTGCGCTCCTCTAATTG | 1562 | TCTGCCCCTTGGTTTCCTTC | 1557 |
| HMGA1 | GCTCTTTTTAAGCTCCCCTG | 1568 | TCCTCCAGTGAGGAGCAGG | 1567 |
| HMGA1 | GCCCCTGGGTCGCTCTTTT | 1570 | AGCTGTCCAGTCCCAGAAGG | 1695 |
| HMGA1 | GTCCTCAGCGCCCAGCAC | 1546 | AGATGCCCTCCTCTTCCTCC | 1681 |
| HMGA1 | CTGGCGCGGCTCCAAGAAG | 1538 | TCACTGCTCCTCCTCCGAGGACTC | 1661 |
| HMGA1 | GCCGGGGCTATTTCTGGC | 1588 | TCTGCCCCTTGGTTTCCTTC | 1557 |
| HMGA1 | TTTCAGATCCGCATTTGCTAC | 1702 | GAAGGAAGCTGCTCCTCCAG | 1703 |
| HMGA1 | ATCCCAGCCATCACTCTTCCACCT | 1520 | CCTCCGAGGACTCCTGCGAG | 1539 |
| HMGA1 | GTCCTCAGCGCCCAGCAC | 1546 | CTGGGGAAGGGGTGGGGG | 1645 |
| HMGA1 | ATCCGCATTTGCTACCAGC | 1548 | TCCTCCAGTGAGGAGCAGG | 1567 |
| HMGA1 | GCTCTTTTTAAGCTCCCCTG | 1568 | AGCAAAGCTGTCAGTCCC | 1613 |
| HMGA1 | GGGTTTAGCCCTAGCCGCTA | 1712 | CAAAGCTGTCCAGTCCCAGAAGG | 1619 |
| HMGA1 | GCCTCGGGGGTTTAGCCCTA | 1714 | GGTGGGAGCGGAGCAAAGCT | 1641 |
| HMGA1 | CTGGAGCCTGATGCCTCG | 1716 | ATGGTGGGCCTGGGGAAG | 1643 |
| HMGA1 | CCTGATGCCTCGGGGGTTTA | 1718 | CTGGGGAAGGGGTGGGGG | 1645 |
| HMGA1 | CGGGTCTGGAGCCTGATG | 1720 | GGTGGTGATGGTGGGCCT | 1721 |
| HMGA1 | ATCACTCTTCCACCTGCTCC | 1722 | TGTGTAGTGTGGTGGTGAGGG | 1617 |
| HSC20 | TCAGAGAAGCATTCGACCCT | 1724 | TTGTCATCCACCCACCTCA | 1725 |
| HSC20 | TCAGAGAAGCATTCGACCCT | 1724 | CTTGTTCAAAAGCACTGCTCAC | 1727 |
| HSC20 | AAGCATTCGACCCTGGTGAA | 1728 | AAAGCACTGCTCACATTGTCAG | 1729 |
| HSC20 | TTCGACCCTGGTGAATGATG | 1730 | CACTGCTCACATTGTCAGTAAATTC | 1731 |
| HSC20 | CCTGAGCAGAGGACTGTACCTT | 1732 | TTGTCATCCACCCACCTCA | 1725 |
| HSC20 | GCCTATAAGACCCTCCTGGC | 1734 | AAATTTCCTTGGCTTCTTCAAAG | 1735 |
| HSC20 | GAATGATGCCTATAAGACCCTCC | 1736 | GTGCCCAGCAAGAACTTTATTT | 1737 |
| HSC20 | ATACAGCGAAGCTCCAGCAC | 1738 | CATCTTTGTCAAAATTTCCTTGG | 1739 |
| HSC20 | TCCTTCAGAGTTGATACAGCGA | 1740 | TTGTCAAAATTTCCTTGGCTTCTT | 1741 |
| HSC20 | CAACCGTTCCTTCAGAGTTGA | 1742 | ATTTGAAAAGTATCTCATCTTTGTCAA | 1743 |
| HSC20 | CCCACTCGAGACTACTTCAGC | 1744 | TCCACAATTAAAGGGGAATCTTC | 1745 |
| HSC20 | TTCTTCAGCCAGAGGTCTCAG | 1746 | TCCACAATTAAAGGGGAATCTTC | 1745 |
| HSC20 | ACCTGACCCCACTCGAGACT | 1747 | AACTTTAAACTATCCACAATTAAAGGG | 1748 |
| HSC20 | CCAGATTTCTTCAGCCAGAGG | 1749 | CTTTAAACTATCCACAATTAAAGGGG | 1750 |
| HSC20 | GACCCTGGTGAATGATGCCTATA | 1751 | CTCACATTGTCAGTAAATTCTTTCTGT | 1750 |
| HSC20 | CCTGAGCAGAGGACTGTACCTT | 1732 | TCCACAATTAAAGGGGAATCTTC | 1745 |
| HSC20 | CTATAAGACCCTCCTGGCCC | 1755 | CATCTTTGTCAAAATTTCCTTGG | 1739 |
| HSC20 | TCCTTCAGAGTTGATACAGCGA | 1740 | AAATTTCCTTGGCTTCTTCAAAG | 1735 |
| HSC20 | ATACAGCGAAGCTCCAGCAC | 1738 | TTGTCAAAATTTCCTTGGCTTCTT | 1741 |
| HSC20 | AGAGTTGATACAGCGAAGCTCC | 1761 | CTTTAAACTATCCACAATTAAAGGGG | 1750 |
| HSC20 | TTCTTCAGCCAGAGGTCTCAG | 1746 | CTTTAAACTATCCACAATTAAAGGGG | 1750 |
| HSC20 | CGTCTTGTCCACCCAGATTT | 1764 | GGGAATCTTCTTTAACTTGATCTTTTC | 1765 |
| HSC20 | TCAGAGAAGCATTCGACCCT | 1724 | CCTGTCCATTTCATAATCTGTCC | 1767 |
| HSC20 | GCCTATAAGACCCTCCTGGC | 1734 | CAGCTTCACTTTCAGCTTCTGC | 1769 |
| HSC20 | TTCGACCCTGGTGAATGATG | 1730 | TTCTATGAGGAATTGCCTGTCC | 1771 |
| HSC20 | CCTGAGCAGAGGACTGTACCTT | 1732 | TTCAATCTCTTTCATGGCAGC | 1773 |
| HSC20 | GAATGATGCCTATAAGACCCTCC | 1736 | CACTTTCAGCTTCTGCGAGTTT | 1775 |
| HSC20 | AAGCATTCGACCCTGGTGAA | 1728 | GGAATTGCCTGTCCATTTCA | 1777 |
| HSC20 | GACCCTGGTGAATGATGCCTATA | 1751 | CCATTATTTCTATGAGGAATTGCC | 1779 |
| HSC20 | AGAGTTGATACAGCGAAGCTCC | 1761 | TGTCCTTTCAGGAATCTCTATTCC | 1781 |
| HSC20 | CAACCGTTCCTTCAGAGTTGA | 1742 | CTTTCATGGCAGCTTCACTTTC | 1783 |
| HSC20 | TCCTTCAGAGTTGATACAGCGA | 1740 | CAGGAATCTCTATTCCATGGAGC | 1785 |
| HSC20 | ATACAGCGAAGCTCCAGCAC | 1786 | TTTGACAATGGATTCAATCTCTTTC | 1787 |
| HSC20 | AGTTGATACAGCGAAGCTCCAGCACAG | 1788 | CAATGGATTCAATCTCTTTCATGGCAG | 1789 |
| HSC20 | CGTCTTGTCCACCCAGATTT | 1764 | CCATTTCATAATCTGTCCTTTCAGG | 1791 |
| HSC20 | CCAGATTTCTTCAGCCAGAGG | 1749 | TGCGAGTTTTTCATTGATTTCC | 1793 |
| HSC20 | AGCAACTGCAGCGTCTTGTC | 1794 | CATAATCTGTCCTTTCAGGAATCTCT | 1795 |
| HSC20 | AAGCTCCAGCACAGGTACCA | 1796 | AGCTTCTGCGAGTTTTTCATTG | 1797 |
| HSC20 | GGGAGACTGGAAGACTTGAATG | 1798 | CACTGCTCACATTGTCAGTAAATTC | 1731 |
| HSC20 | GACTGGAAGACTTGAATGAATAGG | 1800 | CTCACATTGTCAGTAAATTCTTTCTGT | 1750 |
| HSC20 | GGCCTGGGAGACTGGAAGAC | 1802 | AAATTTCCTTGGCTTCTTCAAAG | 1735 |
| HSC20 | GTACTTGAGGGGCAGGGC | 1804 | CATCTTTGTCAAAATTTCCTTGG | 1739 |
| HSC20 | GGCAGGGCCTGGGAGACTG | 1806 | CTTTGTCAAAATTTCCTTGGCTTCTTC | 1807 |
| HSC20 | AGCAACTGCAGCGTCTTGTC | 1794 | ATTTGAAAAGTATCTCATCTTTGTCAA | 1809 |
| IGSF4 | CACCACCATCCTTACCATCATC | 1810 | AGAATGATGAGCAAGCACAGC | 1811 |
| IGSF4 | CGACGACAGAACCAGCAGTT | 1812 | AGAATGATGAGCAAGCACAGC | 1811 |
| IGSF4 | ACACAACGGCGACGACAGAA | 1813 | AAGCACAGCATGGCGAACAC | 1814 |
| IGSF4 | CACCACCATCCTTACCATCATC | 1810 | CAAAATAGCGCCCCAGAATG | 1816 |
| IGSF4 | ACAACTATCCCTCCTCCCACA | 1817 | ATCGAGCCTTCTTCACCTGC | 1818 |
| IGSF4 | ATCCCTCCTCCCACAACAAC | 1819 | CATGATCCACTGCCCTGATC | 1820 |
| IGSF4 | ACCACCACCACCATCCTTAC | 1821 | TTATGTCTGGCAAAATAGCGC | 1822 |
| IGSF4 | CCCCCACAACTATCCCTCCT | 1823 | TTCTTCACCTGCTCGGGAAT | 1824 |
| IGSF4 | CCTCCCACAACAACCACCAC | 1825 | ATGAGCAAGCACAGCATGGC | 1826 |
| IGSF4 | CCCAACCTGTTCATCAATAACC | 1827 | CCCTGATCGAGCCTTCTTC | 1828 |
| IGSF4 | ATGGTAACTTGGGTGAGAGTCG | 1829 | CACCGATCACGGCATGAT | 1830 |
| IGSF4 | ATGGTACATACCGCTGTGAAGC | 1831 | GCCCCAGAATGATGAGCAAG | 1832 |
| IGSF4 | ATGAAATGCCTCAACACGCC | 1833 | CACTGCCCTGATCGAGCCTT | 1834 |
| IGSF4 | TGGGGAAAGCTCACTCGGATTA | 1835 | AATAGCGCCCCAGAATGATGAGC | 1836 |
| IGSF4 | CTTCAAACATAGTGGGGAAAGC | 1837 | GCTCCTTTGGCTTCATGAGT | 1838 |
| IGSF4 | GCTCACTCGGATTATATGCTGTATG | 1839 | TTATAGCTGTGTCTGCGTCTGC | 1840 |
| IGSF4 | AACATAGTGGGGAAAGCTCACTC | 1841 | GTCATCGGCTCCTTTGGCTT | 1842 |
| IGSF4 | TGTGAAGCTTCAAACATAGTGGG | 1843 | TGCGTCTGCTGCGTCATC | 1844 |
| IGSF4 | ATACCGCTGTGAAGCTTCAAACATAGT | 1845 | GCTGCGTCATCGGCTCCTTT | 1846 |
| IGSF4 | CAGATAATGGTACATACCGCTGTG | 1847 | CCTCCTTCTGCATTGATTATAGC | 1848 |
| IGSF4 | AACAAAACAGATAATGGTACATACCG | 1849 | CATTGATTATAGCTGTGTCTGCG | 1850 |
| IGSF4 | TCTGGGCCCAACCTGTTCAT | 1851 | GTGTCTGCGTCTGCTGCGTC | 1852 |
| IGSF4 | GTACTGTCTGGGCCCAACCT | 1853 | CCTTCTGCATTGATTATAGCTGTGTCT | 1854 |
| IGSF4 | CCTCCCACAACAACCACCAC | 1825 | AAGCACAGCATGGCGAACAC | 1814 |
| IGSF4 | CTTCAAACATAGTGGGGAAAGC | 1857 | ATCACGGCATGATCCACTG | 1858 |
| IGSF4 | ATGGTACATACCGCTGTGAAGC | 1831 | ATGGCGAACACCACCACC | 1860 |
| IGSF4 | TGGGGAAAGCTCACTCGGATTA | 1835 | CACGACGCCACCGATCAC | 1862 |
| IGSF4 | AACAAAACAGATAATGGTACATACCG | 1849 | CCTTTGGCTTCATGAGTGAAG | 1864 |
| IGSF4 | GTACATACCGCTGTGAAGCTTCAAAC | 1865 | GCTGCGTCATCGGCTCCTTT | 1846 |
| IGSF4 | TCAACACGCCGTACTGTCTG | 1867 | TTGGCTTCATGAGTGAAGTATGTAC | 1868 |
| IGSF4 | ATGCCTCAACACGCCGTACT | 1869 | CGGCTCCTTTGGCTTCATGA | 1870 |
| IGSF4 | GAGTCGATGATGAAATGCCTC | 1871 | TTCGGAGTCGTTCTGTCCTC | 1872 |
| IGSF4 | ACGCCGTACTGTCTGGGCCC | 1873 | TCTTCGGAGTTGTTCTGTCCTCCTTCT | 1874 |
| IGSF4 | AACAAAACAGATAATGGTACATACCG | 1849 | GCTCCTTTGGCTTCATGAGT | 1838 |
| KITLG | TGATGCCTTCAAGGACTTTGT | 1877 | CTGCCCTTGTAAGACTTGGC | 1878 |
| KITLG | CCAGGCTCTTTACTCCTGAAGA | 1879 | TCCAGTATAAGGCTCCAAAAGC | 1880 |
| KITLG | TCATTCAAGAGCCCAGAACC | 1881 | CTCCAAAAGCAAAGCCAATT | 1882 |
| KITLG | GTGTGGTTTCTTCAACATTAAGTCC | 1883 | TAAGGCTCCAAAAGCAAAGC | 1884 |
| KITLG | CAAGGACTTTGTAGTGGCATCTG | 1885 | GAAAACAATGCTGGCAATGC | 1886 |
| KITLG | GCATCTGAAACTAGTGATTGTGTGG | 1887 | ATAAGAGAAAACAATGCTGGCAA | 1888 |
| KITLG | GATCCATTGATGCCTTCAAGG | 1889 | CCAAAAGCAAAGCCAATTATAAGAG | 1890 |
| KITLG | CCAGAACCCAGGCTCTTTACT | 1891 | GCCCAGTGTAGGCTGGAGTC | 1892 |
| KITLG | GGCTCTTTACTCCTGAAGAATTCTTT | 1893 | CCATGGCTGCCCAGTGTAG | 1894 |
| KITLG | AAGAGCCCAGAACCCAGGCT | 1895 | AGGGGGATTTTTGGCCTTC | 1896 |
| KITLG | GGACTTTGTAGTGGCATCTGAAACTAG | 1897 | AATGCTGGCAATGCCATGG | 1898 |
| KITLG | TGCCTTCAAGGACTTTGTAGTGGC | 1899 | CAATGCCATGGCTGCCCAGT | 1900 |
| KITLG | AAAATCATTCAAGAGCCCAGAACCCAG | 1901 | GTATAAGGCTCCAAAAGCAAAGCCAAT | 1902 |
| KITLG | GTGATTGTGTGGTTTCTTCAACA | 1903 | CTGCCCTTGTAAGACTTGGC | 1878 |
| KITLG | GAAACTAGTGATTGTGTGGTTTCTTC | 1905 | CCTTGTAAGACTTGGCTGTCTCTT | 1906 |
| KITLG | TTCTTCAACATTAAGTCCTGAGAAAG | 1907 | TTTGTATATTTTCAACTGCCCTTG | 1908 |
| KITLG | GTGGAGTGCGTGAAAGAAAACTCATC | 1909 | CAACTGCCCTTGTAAGACTTGGCTGTC | 1910 |
| KITLG | TCCCCGGGATGGATGTTTTG | 1911 | GTCTCCAGGGGGATTTTTGGCCTT | 1912 |
| LGALS9 | GTGATGGTGAACGGGATCCT | 1913 | GTTGGCAGGCCACACGCC | 1914 |
| LGALS9 | ACGGGATCCTCTTCGTGCAGTA | 1915 | GTTGGCAGGCCACACGCC | 1914 |
| LGALS9 | GGATCCTCTTCGTGCAGTACTTCCAC | 1916 | AATGGGAGCCGGGTTGGC | 1917 |
| LGALS9 | GTGATGGTGAACGGGATCCT | 1913 | GCTCTGCACTGTGTGGATGA | 1919 |
| LGALS9 | GACACCATCTCCGTCAATGG | 1920 | AGAGAACATCTGTCCAGGGG | 1921 |
| LGALS9 | AGTACTTCCACCGCGTGC | 1922 | TGTGTGGATGACTGTCTGGG | 1923 |
| LGALS9 | GGTGAACGGGATCCTCTTCG | 1924 | CTGCACTGTGTGGATGACTGTCT | 1925 |
| LGALS9 | TTCCACCGTGTGGACACCAT | 1926 | ATCTGTCCAGGGGCGCTCTG | 1927 |
| LGALS9 | CCGTGTGGACACCATCTCCG | 1928 | AGGGGCGCTCTGCACTGTGT | 1929 |
| LGALS9 | AATGGCTCTGTGCAGCTGTC | 1930 | GGGTACATCATAGGTGGGATGG | 1931 |
| LGALS9 | GCTGTCCTACATCAGCTTCCA | 1932 | GGGGTGGGGGTACATCATAG | 1933 |
| LGALS9 | TCTCCGTCAATGGCTCTGTG | 1934 | ATAGGTGGGATGGCGGGAGT | 1935 |
| LGALS9 | GGCTCTGTGCAGCTGTCCTACAT | 1936 | ATAGGCGGGGTGGGGGTACAT | 1937 |
| LGALS9 | GTGCCCTTCCACCGTGTG | 1938 | GGTACAGCCCTCCCAGAATG | 1939 |
| LGALS9 | TGTGCAGCTGTCCTACATCAGCTT | 1940 | GGTGGTGATGAAAGGCATCG | 1941 |
| LGALS9 | CTTCCACCGCGTGCCCTTC | 1942 | CCCTCCCAGAATGGTGGTGAT | 1943 |
| LGALS9 | CTGGTGCAGAGCTCAGATTTC | 1944 | CAGAATGGTGGTGATGAAAGG | 1945 |
| LGALS9 | CTTTGACCTCTGCTTCCTGG | 1946 | TGGACTTGGATGGGTACAGC | 1947 |
| LGALS9 | GCTTCCTGGTGCAGAGCTC | 1948 | AGGAGGATGGACTTGGATGG | 1949 |
| LGALS9 | ACCTCTGCTTCCTGGTGCAG | 1950 | TTGGATGGGTACAGCCCTCC | 1951 |
| LGALS9 | ATGCCCTTTGACCTCTGCTT | 1952 | CTGACAGGAGGATGGACTTG | 1953 |
| LGALS9 | ACACATGCCTTTCCAGAAGG | 1954 | AGGACAGTGCCTGACAGGAG | 1955 |
| LGALS9 | TTTCCAGAAGGGGATGCC | 1956 | AGTGCCTGACAGGAGGATGG | 1957 |
| LGALS9 | AGGAGAGGAAGACACACATGC | 1958 | CACTGGGCAGGACAGTGC | 1959 |
| LGALS9 | AGGGGATGCCCTTTGACCTC | 1960 | TGGGCAGGACAGTGCCTGAC | 1961 |
| LGALS9 | GGAAGACACACATGCCTTTCC | 1962 | CTGAGCACTGGGCAGGACAG | 1963 |
| MCL1 | CCAAGGACACAAAGCCAATG | 1964 | TGGAAGAACTCCACAAACCC | 1965 |
| MCL1 | TGGAGACCTTACGACGGGTT | 1966 | TACTCCAGCAACACCTGCAA | 1967 |
| MCL1 | GAAGGCGCTGGAGACCTTAC | 1968 | CACATTCCTGATGCCACCTT | 1969 |
| MCL1 | GCAGCGCAACCACGAGAC | 1970 | CAAACCAGCTCCTACTCCAGC | 1971 |
| MCL1 | GACACAAAGCCAATGGGCAG | 1972 | AGGTCCTCTACATGGAAGAACTCC | 1973 |
| MCL1 | ACGAGACGGCCTTCCAAG | 1974 | TTAGATATGCCAAACCAGCTCC | 1975 |
| MCL1 | CTTACGACGGGTTGGGGATG | 1976 | ACACCTGCAAAAGCCAGCAG | 1977 |
| MCL1 | AAAGCCAATGGGCAGGTCTG | 1978 | TTCCTGATGCCACCTTCTAGGT | 1979 |
| MCL1 | TTATCTCTCGGTACCTTCGGG | 1980 | TCTACATGGAAGAACTCCACAAAC | 1981 |
| MCL1 | CAACCACGAGACGGCCTT | 1982 | ATATGCCAAACCAGCTCCTACTCC | 1983 |
| MCL1 | GTCTGGGGCCACCAGCAG | 1984 | AAGCCAGCAGCACATTCCTG | 1985 |
| MCL1 | AGCAGGAAGGCGCTGGAGAC | 1986 | CAGCAACACCTGCAAAAGCC | 1987 |
| MCL1 | GTACCTTCGGGAGCAGGC | 1988 | CCTTCTAGGTCCTCTACATGGAAG | 1989 |
| MCL1 | GGGCCACCAGCAGGAAGG | 1990 | TGATGCCACCTTCTAGGTCCTCTA | 1991 |
| MCL1 | ATGGGCAGGTCTGGGGCC | 1992 | CAGCAGCACATTCCTGATGCCA | 1993 |
| MCL1 | CGGCGCCAAGGACACAAAG | 1994 | TGCAAAAGCCAGCAGCACAT | 1995 |
| MCL1 | GTACCGGCAGTCGCTGGAGATTA | 1996 | CAGCACATTCCTGATGCCACCTTCTAG | 1997 |
| NRG1 | GTTTACTGGTGATCGCTGCC | 1998 | TGGGCTGTGGAAGTATAGTGAC | 1999 |
| NRG1 | GTTTACTGGTGATCGCTGCC | 1998 | ACCACACACATGATGCCGAC | 2000 |
| NRG1 | GTGCCCAAATGAGTTTACTGG | 2001 | ACATGATGCCGACCACAAG | 2002 |
| NRG1 | GATCGCTGCCAAAACTACGT | 2003 | TAGGCCACCACACACATGAT | 2004 |
| NRG1 | CCAAATGAGTTTACTGGTGATCG | 2005 | TTGGTTTTGCAGTAGGCCAC | 2006 |
| NRG1 | CTGCCAAAACTACGTAATGGC | 2007 | TTTGCAGTAGGCCACCACAC | 2008 |
| NRG1 | AATGAGTTTACTGGTGATCGCTGCCAA | 2009 | CACAAGGAGGGCGATGCAGAT | 2010 |
| NRG1 | ACTGGTGATCGCTGCCAAAACTAC | 2011 | GATGCCGACCACAAGGAGGG | 2012 |
| NRG1 | GTGATCGCTGCCAAAACTACGTAATGG | 2013 | GGCGATGCAGATGCCGGTTAT | 2014 |
| NRG1 | CTGGGACAAGCCATCTTGTAA | 2015 | TATGGTCAGCACTCTCTTCTGG | 2016 |
| NRG1 | GAGTGCTTCATGGTGAAAGACC | 2017 | TCTCTTCTGGTACAGCTCCTCC | 2018 |
| NRG1 | AACTTTCTGTGTGAATGGAGGG | 2019 | AGATGCCGGTTATGGTCAGC | 2020 |
| NRG1 | TACATCTACATCCACCACTGGG | 2021 | TCAGCACTCTCTTCTGGTACAGC | 2022 |
| NRG1 | AACCCCTCGAGATACTTGTGC | 2023 | CCGGTTATGGTCAGCACTCT | 2024 |
| NRG1 | ATCTACATCCACCACTGGGACAAGCC | 2025 | AGATGCCGGTTATGGTCAGCACTCTCT | 2026 |
| NRG1 | ACGTAATGGCCAGCTTCTACA | 2027 | GGTGGGTTAGGATGGTGAGG | 2028 |
| NRG1 | AAAACTACGTAATGGCCAGCTTC | 2029 | TGTTTCGTTCAGACCGAAGG | 2030 |
| NRG1 | GTGCGGAGAAGGAGAAAACTT | 2031 | GAAGACGGTCATGCAGCTTT | 2032 |
| NRG1 | AAGACCTTTCAAACCCCTCG | 2033 | CCCATTGGCAATGTTCATC | 2034 |
| NRG1 | CTTGTAAAATGTGCGGAGAAGG | 2035 | GCAATGTTCATCATATTGTTTCG | 2036 |
| NRG1 | GTGAATGGAGGGGAGTGCTT | 2037 | TTAGGATGGTGAGGCCCATT | 2038 |
| NRG1 | CAAGCCATCTTGTAAAATGTGC | 2039 | TCATCATATTGTTTCGTTCAGACC | 2040 |
| NRG1 | CCTTTCAAACCCCTCGAGATAC | 2041 | GCAGCTTTTTCCGCTGTTTC | 2042 |
| NRG1 | CATGGTGAAAGACCTTTCAAACC | 2043 | AAGGCTCTGCCGAAGACG | 2044 |
| NRG1 | GAGGGGAGTGCTTCATGGT | 2045 | TCACCAGCTGGACATTCTCG | 2046 |
| NRG1 | TAAAATGTGCGGAGAAGGAGAAAA | 2047 | CGTTCAGACCGAAGGCTCTG | 2048 |
| NRG1 | TTCTGTGTGAATGGAGGGGAGTGC | 2049 | TGAGGCCCATTGGCAATGTT | 2050 |
| NRG1 | AGGAGAAAACTTTCTGTGTGAATGGAG | 2051 | GACCGAAGGCTCTGCCGAAG | 2052 |
| NRG1 | CTCCCATTAGAATATCAGTATCCACAG | 2053 | GTCATGCAGCTTTTTCCGCT | 2054 |
| NRG1 | ATGCCAGCCTCAACTGAAGG | 2055 | ATATTGTTTCGTTCAGACCGAAGGCTC | 2056 |
| NRG1 | TCCACAGAAGGAGCAAATACTTC | 2057 | CTGGAGATGACGTTTTTAGATACG | 2058 |
| NUP98 | TCGTATCTGGAGGGTTCTGG | 2059 | TCAGATTCCATGTGTGCTCG | 2060 |
| NUP98 | TCGTATCTGGAGGGTTCTGG | 2059 | TCTCGAACAGCCTTCTCACG | 2062 |
| NUP98 | CGAGATGTCTGCTTTCACCTTC | 2063 | TCAGATTCCATGTGTGCTCG | 2060 |
| NUP98 | ACTCACAGACACCACTTCGAGA | 2065 | TCTCTTTAGCCCAAGATTCAGG | 2066 |
| NUP98 | TGTGATAGCGGAGGAGCAAA | 2067 | TCTGGGTAAGGAAAGTCTCTTTAGC | 2068 |
| NUP98 | CCCACTTCCTTCGTATCTGG | 2069 | GGGTAAGCAGCTCTCGAACA | 2070 |
| NUP98 | GAGGGTTCTGGCTGTGTGATA | 2071 | CAAGATTCAGGGGTCTCCAA | 2072 |
| NUP98 | ACACCACTTCGAGATGTCTGC | 2073 | ATCCATTTGGCAGGTACACG | 2074 |
| NUP98 | AGGAGCAAAACTCACAGACACC | 2075 | GTACACGGAGCTTCTGGGTAAG | 2076 |
| NUP98 | CTGGCTGTGTGATAGCGGAG | 2077 | AGGAAAGTCTCTTTAGCCCAAGA | 2078 |
| NUP98 | TGACAGTGACAGATATGCCTGC | 2079 | AGCAGCTCTCGAACAGCCTT | 2080 |
| NUP98 | CTTCCTTCGTATCTGGAGGGTT | 2081 | GTCTCCAACAGCTGGCAGTG | 2082 |
| NUP98 | ATAGCGGAGGAGCAAAACTCAC | 2083 | GGAGCTTCTGGGTAAGGAAAGT | 2084 |
| NUP98 | CTGCTTTCACCTTCTAAAACTCTACAG | 2085 | GCCTCGTGGATCCATTTG | 2086 |
| NUP98 | CCACTTCGAGATGTCTGCTTTCAC | 2087 | TCGTGGATCCATTTGGCAGG | 2088 |
| NUP98 | TATCTGGAGGGTTCTGGCTGTGT | 2089 | AGTGCCGGGTAAGCAGCTCT | 2090 |
| NUP98 | ATATGCCTGCTCCCCACTTC | 2091 | TTCAGGGGTCTCCAACAGCT | 2092 |
| NUP98 | CCTGCTCCCCACTTCCTTCGTA | 2093 | AGCTCTCGAACAGCCTTCTCACGTATG | 2094 |
| NUP98 | GAGCAAAACTCACAGACACCACTTCGA | 2095 | CATTTGGCAGGTACACGGAGCTT | 2096 |
| NUP98 | CTGTGTGATAGCGGAGGAGCAAAACTC | 2097 | AGCTGGCAGTGCCGGGTAAG | 2098 |
| NUP98 | GACAGATATGCCTGCTCCCC | 2099 | TTAGCCCAAGATTCAGGGGTCTC | 2100 |
| NUP98 | AATACCTCTGACAGTGACAGATATGC | 2101 | GCTTTGGCCTCGTGGATC | 2102 |
| NUP98 | ACTTGTGGGAAGTGCTGAGG | 2103 | TCAGATTCCATGTGTGCTCG | 2060 |
| NUP98 | TCGAAGCATAACAGCAGATCC | 2105 | TCTCTTTAGCCCAAGATTCAGG | 2066 |
| NUP98 | TAACTACACCCATCTCTCAGCG | 2107 | ATCCATTTGGCAGGTACACG | 2074 |
| NUP98 | ATCCTTTGGACTACCGCCTAA | 2109 | GGAGCTTCTGGGTAAGGAAAGT | 2084 |
| NUP98 | CATTATGATCTCAACCAGCTGC | 2110 | TCTCGAACAGCCTTCTCACG | 2062 |
| NUP98 | TAAGCTGGCACTTGTGGGAA | 2113 | TCTGGGTAAGGAAAGTCTCTTTAGC | 2068 |
| NUP98 | ACAGCAGATCCTTTGGACTACC | 2115 | GGGTAAGCAGCTCTCGAACA | 2070 |
| NUP98 | AGTGCTGAGGGCTCTTAACTACAC | 2117 | GTACACGGAGCTTCTGGGTAAG | 2076 |
| NUP98 | GGCTCTTAACTACACCCATCTCTC | 2119 | AGGAAAGTCTCTTTAGCCCAAGA | 2078 |
| NUP98 | ATCTCTCAGCGCAGTGTGAAG | 2121 | GCCTCGTGGATCCATTTG | 2086 |
| NUP98 | ACTACCGCCTAAGCTGGCAC | 2123 | CAAGATTCAGGGGTCTCCAA | 2072 |
| NUP98 | GAGCCTCGAAGCATAACAGC | 2125 | GTCTCCAACAGCTGGCAGTG | 2082 |
| NUP98 | AGCATAACAGCAGATCCTTTGGA | 2127 | AGTGCCGGGTAAGCAGCTCT | 2090 |
| NUP98 | TGGGAAGTGCTGAGGGCTCT | 2129 | CATTTGGCAGGTACACGGAGCTT | 2096 |
| NUP98 | ACACCCATCTCTCAGCGCAGTGT | 2131 | TCGTGGATCCATTTGGCAGG | 2088 |
| NUP98 | TGCTGGAGCCTCGAAGCATA | 2133 | AGCAGCTCTCGAACAGCCTT | 2080 |
| NUP98 | CTTTGGACTACCGCCTAAGCTGGC | 2135 | AGCTGGCAGTGCCGGGTAAG | 2098 |
| NUP98 | ACTTGTGGGAAGTGCTGAGGGCTCTTA | 2137 | TGAGCTTGTGGCAGCGGTTC | 2138 |
| NUP98 | CGAGATGTCTGCTTTCACCTTC | 2063 | GCTTTGGCCTCGTGGATC | 2102 |
| NUP98 | CTGCTTTCACCTTCTAAAACTCTACAG | 2085 | ATGTGTGCTCGCACAGCTTT | 2142 |
| NUP98 | ACACCACTTCGAGATGTCTGC | 2143 | GCACAGCTTTGGCCTCGT | 2144 |
| NUP98 | CCTGCTCCCCACTTCCTTCGTAT | 2145 | AGCTCTCGAACAGCCTTCTCACGTATG | 2094 |
| NUP98 | ACTCACAGACACCACTTCGAGA | 2147 | AGTGCTTGTCAGATTCCATGTG | 2148 |
| NUP98 | AGGAGCAAAACTCACAGACACC | 2075 | GGCCTCTAAGTGCTTGTCAGAT | 2150 |
| NUP98 | GAGCAAAACTCACAGACACCACTTCGA | 2095 | GGTAAGCAGCTCTCGAACAGCCTTCTC | 2152 |
| NUP98 | CCACTTCGAGATGTCTGCTTTCAC | 2087 | AGCCTTAAATAAGCAAAGGGCC | 2154 |
| NUP98 | TGTGATAGCGGAGGAGCAAA | 2067 | TAAGCAAAGGGCCTCTAAGTGC | 2156 |
| NUP98 | CTGTGTGATAGCGGAGGAGCAAAACTC | 2157 | AGCTTTGGCCTCGTGGATCCATTT | 2158 |
| NUP98 | ATAGCGGAGGAGCAAAACTCACAGACA | 2159 | TGCTCGCACAGCTTTGGCCT | 2160 |
| NUP98 | TTCTGGCTGTGTGATAGCGG | 2161 | CCTTAAATAAGCAAAGGGCCTCTAAGT | 2162 |
| NUP98 | TCCCCACTTCCTTCGTATCTGGAG | 2163 | GATTCCATGTGTGCTCGCACAGC | 2164 |
| NUP98 | GACAGATATGCCTGCTCCCC | 2099 | TCTCTTTAGCCCAAGATTCAGG | 2066 |
| NUP98 | GACAGTGACAGATATGCCTGCTC | 2167 | TCTGGGTAAGGAAAGTCTCTTTAGC | 2068 |
| NUP98 | GGAGGGTTCTGGCTGTGTGATAGC | 2169 | TCGTGGATCCATTTGGCAGG | 2088 |
| NUP98 | AGTGACAGATATGCCTGCTCCCCACTT | 2171 | TGCTCGCACAGCTTTGGCCT | 2160 |
| NUP98 | ATTTGCTTCCACCAACAGCC | 2173 | CAAGATTCAGGGGTCTCCAA | 2072 |
| NUP98 | CGCTCAGCATGTATGAAGAAGC | 2175 | AGGAAAGTCTCTTTAGCCCAAGA | 2078 |
| NUP98 | CAGCATGTATGAAGAAGCATTTCAG | 2177 | TTAGCCCAAGATTCAGGGGTCTC | 2100 |
| NUP98 | CTTCCACCAACAGCCTCCATTT | 2179 | AGCTGGCAGTGCCGGGTAAG | 2098 |
| NUP98 | GGAAACGCTCCCTGGCTATC | 2181 | GTAAGCAGCTCTCGAACAGCCTTC | 2182 |
| NUP98 | ACAGCCTCCATTTCTAGGGC | 2183 | GGCCTCTAAGTGCTTGTCAGAT | 2150 |
| NUP98 | CCTCCATTTCTAGGGCGCTCAG | 2185 | GATTCCATGTGTGCTCGCACAGC | 2164 |
| NUP98 | CATTTCTAGGGCGCTCAGCATGTA | 2187 | AGCAAAGGGCCTCTAAGTGCTTGTCAG | 2188 |
| PAXIP1 | GGCACACGTTTCTCCACTCT | 2189 | GATAACACCTTTCCTCCTGCAC | 2190 |
| PAXIP1 | TGAGCAGAACTACATTCTCCGA | 2191 | CATAGTGGAAAGACTTGGGCAG | 2192 |
| PAXIP1 | CTCTTTCAGCTTGGAAGAATCC | 2193 | GCACACTCTACGATTGCCTTC | 2194 |
| PAXIP1 | TACATTCTCCGAGATGCTGAGG | 2195 | CGATTGCCTTCATAGTGGAAAG | 2196 |
| PAXIP1 | ATCCTTAAAACGGGCACACG | 2197 | TGCTTGGATAACACCTTTCCTC | 2198 |
| PAXIP1 | CAGAACTACATTCTCCGAGATGCT | 2199 | TTTCCTCCTGCACACTCTACG | 2200 |
| PAXIP1 | TGGAAGAATCCTTAAAACGGG | 2201 | TTCTGCTTGTGCTCCATGAG | 2202 |
| PAXIP1 | AAACGGGCACACGTTTCTC | 2203 | GGAAAGATGGCTGCTTGGAT | 2204 |
| PAXIP1 | GGCAGAAGTACTTTTCTCTTTCAGC | 2205 | ACTCTACGATTGCCTTCATAGTGG | 2206 |
| PAXIP1 | TCAGCTTGGAAGAATCCTTAAAAC | 2207 | ATGAGCTTCCGGAAAGATGG | 2208 |
| PAXIP1 | ACTTTTCTCTTTCAGCTTGGAAGA | 2209 | CTTGTGCTCCATGAGCTTCC | 2210 |
| PAXIP1 | CTTAAAACGGGCACACGTTTCTCCAC | 2211 | GCTTCCGGAAAGATGGCTGCTT | 2212 |
| PAXIP1 | CGATTTCTGTCGTGAAGCAC | 2213 | GCAGATTCCAGGTGTGATGTAA | 2214 |
| PAXIP1 | ACATTCTTGGTGGAGAGGTTGC | 2215 | AAAGACTTGGGCAGATTCCAG | 2216 |
| PAXIP1 | AGCAAAGTGACTCGCACCGT | 2217 | GCTCCATGAGCTTCCGGAAA | 2218 |
| PAXIP1 | TGGTGGAGAGGTTGCGGAGT | 2219 | ACTTGGGCAGATTCCAGGTGTGA | 2220 |
| PAXIP1 | TGCACACACCTCATTGCCAG | 2221 | AAGATGGCTGCTTGGATAACACCTTTC | 2222 |
| PAXIP1 | ACACACCTCATTGCCAGCAAAGT | 2223 | CCTCCTGCACACTCTACGATTGCC | 2224 |
| PAXIP1 | TGACGCCAGAGTGGCTGGAA | 2225 | GAGTTCTGCTTGTGCTCCATGAGCTTC | 2226 |
| PAXIP1 | TGCTTCAGGTGTCAGAAGTTCA | 2227 | TCTCGGCATAAATGAAGGTCA | 2228 |
| PAXIP1 | TGGAAGAATGCTTCAGGTGTC | 2229 | TCTGGCAAAATATTCTCGGC | 2230 |
| PAXIP1 | CACATAGTGACGCCAGAGTGG | 2231 | AAATGAAGGTCATTTTCACAGGA | 2232 |
| PAXIP1 | AGTGGCTGGAAGAATGCTTCAGG | 2233 | CGGCATAAATGAAGGTCATTTTCA | 2234 |
| PAXIP1 | TTTCTGTCGTGAAGCACATAGTG | 2235 | GAAGGTCATTTTCACAGGATATTAAAA | 2236 |
| PAXIP1 | GGAAGAATGCTTCAGGTGTCAGAAGTT | 2237 | CTGGCAAAATATTCTCGGCATAAATG | 2238 |
| PAXIP1 | TGACGGCGATTTCTGTCGT | 2239 | CTATGCCTCTGGCAAAATATTCTC | 2240 |
| PAXIP1 | AAGTTCCTGACGGCGATTTC | 2241 | CGAACTCTGCATTGTGAACAT | 2242 |
| PAXIP1 | CCGTGAAGTTCCTGACGG | 2243 | TCAGAACGAACTCTGCATTGTG | 2244 |
| PLD1 | ACGACGCAGATAGCATCAGC | 2245 | TTGCAGTAGTCCTTTCCATGC | 2246 |
| PAXIP1 | CCGCAATGGAGTCTATGGAA | 2247 | CTCTCCCACACCTGTCTGTAAAC | 2248 |
| PAXIP1 | CTGAAAGGAATAGGAAAGCCAAG | 2249 | TCTGTAAACTACGGATGGACCC | 2250 |
| PAXIP1 | TCCAGAAGAGTATTGATGATGTGG | 2251 | CAAGTTGAACCCAGTCTTTGAAG | 2252 |
| PAXIP1 | CCTGTTCAAAACCTACCCATCC | 2253 | AGTCCTTTCCATGCCAGAATC | 2254 |
| PAXIP1 | CTCTACAAGCAGCTCCACAGG | 2255 | TGAAGACGAAATTGCAGTAGTCC | 2256 |
| PAXIP1 | ATCAGCAGCATTGACAGCAC | 2257 | AAAGGTTTATCAAGTTGAACCCAG | 2258 |
| PAXIP1 | AAAGCCAAGAAAGTTCTCCAAA | 2259 | AGAATCTGGTTTCCCCATGC | 2260 |
| PAXIP1 | CAGATAGCATCAGCAGCATTG | 2261 | CCCAGTCTTTGAAGACGAAATTG | 2262 |
| PAXIP1 | ACCTACCCATCCAGAAGAGTATTG | 2263 | GTTTCCCCATGCAGCTCTC | 2264 |
| PAXIP1 | TCCAAATTTAGTCTCTACAAGCAGC | 2265 | CCATGCCAGAATCTGGTTTC | 2266 |
| PAXIP1 | GGAATAGGAAAGCCAAGAAAGTTC | 2267 | ACTACGGATGGACCCGGTAT | 2268 |
| PAXIP1 | TTGATGATGTGGATTCAAAACTG | 2269 | CACCTGTCTGTAAACTACGGATGG | 2270 |
| PAXIP1 | TGGAGTCTATGGAATCCTTAAGACTC | 2271 | ATGCAGCTCTCCCACACCT | 2272 |
| PAXIP1 | AATGAGCCTGTTCAAAACCTACC | 2273 | ACGAAATTGCAGTAGTCCTTTCCA | 2274 |
| PAXIP1 | CACCACCTGCACGACGCAGATA | 2275 | GTCCTTTCCATGCCAGAATCTGGTTTC | 2276 |
| PAXIP1 | GCCAAGAAAGTTCTCCAAATTTAGTC | 2277 | ACTGCAGAGGCAATGTCATG | 2278 |
| PAXIP1 | AGCATTGAGAGCACCTCCA | 2279 | CGCTGGATGAAGTGACGTG | 2280 |
| PAXIP1 | AAGAGTATTGATGATGTGGATTCAAA | 2281 | GGCGTGGAGTACCTGTCAAT | 2282 |
| PAXIP1 | ATTTAGTCTCTACAAGCAGCTCCACAG | 2283 | AATGTCATGCCAGGGCATC | 2284 |
| PAXIP1 | TGGATTCAAAACTGAAAGGAATAGG | 2285 | ATCCGGGGCGTGGAGTAC | 2286 |
| PAXIP1 | CACAGGCACCACCTGCAC | 2287 | ACGTGCCACATCACGAGC | 2288 |
| PAXIP1 | AAAGTTCTCCAAATTTAGTCTCTACAA | 2289 | TTCCCGTGGACTGCAGAG | 2290 |
| PAXIP1 | TTCAAAACCTACCCATCCAGAAGA | 2291 | AGAGGCAATGTCATGCCAGG | 2292 |
| PAXIP1 | AGATAAAAATGAGCCTGTTCAAAACC | 2293 | CATCACGAGCCGCCTTCC | 2294 |
| POLI | AATGGCTCAAACTAAGGACAAGAG | 2295 | TCACGACCATAGTGCTTCTCAG | 2296 |
| POLI | AATGGCTCAAACTAAGGACAAGAG | 2295 | AAAAGACTAGCAAGTAGTTCTTCAATC | 2297 |
| POLI | TGAGTACAATAGCTTGCAGATAAAA | 2298 | AGCTTCAACTTCAGATGAACATTT | 2299 |
| POLI | GAGTCGCCATCTACCTGGTC | 2300 | TCCTCACACACTCCATGCTC | 2301 |
| POLI | CCCAGCTCTGCTGGACATAA | 2302 | TCCTCACACACTCCATGCTC | 2301 |
| POLI | GCTGGACATAAGCTGGTTAACAG | 2303 | CTCTGAGCGCCGAACTCTCT | 2304 |
| POLI | GGACATAAGCTGGTTAACAGAGAGCCT | 2305 | AGCGCCGAACTCTCTCCACC | 2306 |
| POLI | CAGGATGGCAGCTGCTCC | 2307 | TCCACCTCCTCACACACTCC | 2308 |
| POLI | AGGAGCGCAGGATGGCAG | 2309 | ACTCCATGCTCCAGCAGCTC | 2310 |
| POLI | AGCTGCTCCCCCGGGTTG | 2311 | TCACACACTCCATGCTCCAGCAG | 2312 |
| POLI | AGAGAGCCTGGGAGCTGG | 2313 | CTTCATGGTCTGGTACCTCTCTG | 2314 |
| POLI | TGTACCTGTGGAGTGCCG | 2315 | GTCCTGGTGGTGCTGGAG | 2316 |
| POLI | GTTAACAGAGAGCCTGGGAGC | 2317 | TCTGGTACCTCTCTGAGCGC | 2318 |
| POLI | CAGCCTGTACCTGTGGAGTG | 2319 | AGGGCATCTACATCGGACC | 2320 |
| POLI | GGAGCTGGGCAGCCTGTAC | 2321 | TACCTCTCTGAGCGCCGAAC | 2322 |
| POLI | CTGGGCAGCCTGTACCTGT | 2323 | CTACATCGGACCGCAGGACT | 2324 |
| POLI | AGAGGAGGCCGTCAGCTG | 2325 | GGTGCTCAGGTCCTGGTG | 2326 |
| POLI | GCCGTCAGCTGGCAGGAG | 2327 | GCTCAGGTCCTGGTGGTGCT | 2328 |
| PTK2 | TGACAGCTACAACGAGGGTG | 2329 | GATGACAGCTTTCACCAGGC | 2330 |
| PTK2 | TGACAGCTACAACGAGGGTG | 2329 | GATGACAGCTTTCACCAGGC | 2330 |
| PTK2 | AGCTCCACCAAAGAAACCG | 2332 | CCGTCACATTCTCGTACACCT | 2333 |
| PTK2 | GTCATCTGGGAAGCCTTGC | 2334 | CTCGTACACCTTATCATTCGACC | 2335 |
| PTK2 | CCTGCTGACAGCTACAACGA | 2336 | TAGGGACATACTCCTCTGGTGG | 2337 |
| PTK2 | ACCAAAGAAACCGCCTCG | 2338 | TACTGGACATCTCGATGACAGC | 2339 |
| PTK2 | ATCCTGCAGCTCCACCAAAG | 2340 | CTTCACCATAGGGACATACTCCTC | 2341 |
| PTK2 | GCTACAACGAGGGTGTCAAG | 2342 | GCTGGCTGGATTTTACTGGA | 2343 |
| PTK2 | AAGCCTTGCCAGCCTCAG | 2344 | TCACATTCTCGTACACCTTATCATTC | 2345 |
| PTK2 | GAGCTCCCGGTCATCTGG | 2346 | GCTGGATTTTACTGGACATCTCG | 2347 |
| PTK2 | AAGAAACCGCCTCGCCCTG | 2348 | GGACATCTCGATGACAGCTTTCAC | 2349 |
| PTK2 | CTCCCGGTCATCTGGGAAGC | 2350 | TACTCCTCTGGTGGGGCTGG | 2351 |
| PTK2 | TTGCCAGCCTCAGCAGCCCT | 2352 | TTTCACCAGGCCCGTCACATT | 2353 |
| PTK2 | CTGGGAAGCCTTGCCAGCCT | 2354 | CAGGCCCGTCACATTCTCGT | 2355 |
| PTK2 | CCTCGCCCTGGAGCTCCC | 2356 | ACCTTATCATTCGACCGGTCCA | 2357 |
| PTK2 | GCAGCCCTGCTGACAGCTAC | 2358 | GTGGGGCTGGCTGGATTTTA | 2359 |
| PTK2 | TTGGAAACCAACATATATATCAGCC | 2360 | GTCCAGGTTGGCAGTAGGAG | 2361 |
| PTK2 | TATCAGCCTGTGGGTAAACCAG | 2362 | CTGATTTCCTGGGGCTGAAG | 2363 |
| PTK2 | AACCAACATATATATCAGCCTGTGGGT | 2364 | ATTCGACCGGTCCAGGTTGG | 2365 |
| PTK2 | GAGGGATTGGGCAAGTGTTG | 2366 | GGGGGCTGATTTCCTGGG | 2367 |
| PTK2 | GCTGACAGCTACAACGAGGGTGTCAAG | 2368 | CGTGCTCCTAGGGGAGGCTC | 2369 |
| PTK2 | ATGGAAGTCTTCAGGGTCCG | 2370 | AGTTCAATAGCTTCTGTGCCATC | 2371 |
| PTK2 | GAAATGGAAGAAGATCAGCGC | 2372 | AATAATGTCCTCAGGGCCAAG | 2373 |
| PTK2 | GTATTGACAGGGAGGATGGAAG | 2374 | GGTCAGAGTTCAATAGCTTCTGTG | 2375 |
| PTK2 | GCTGGCTGGAAAAAGAGGAA | 2376 | TGGCCAATAATGTCCTCAGG | 2377 |
| PTK2 | AGGGAGGATGGAAGTCTTCAG | 2378 | AGCTCACCCAGGTCAGAGTTC | 2379 |
| PTK2 | AAGATCAGCGCTGGCTGGAA | 2380 | CTCAGGGCCAAGCCGACTTC | 2381 |
| PTK2 | TCTCTCGAGGCAGTATTGACAG | 2382 | CACCCAGGTCAGAGTTCAATAGC | 2383 |
| PTK2 | CAACAGGAAATGGAAGAAGATCA | 2384 | CACAGTGGCCAATAATGTCC | 2385 |
| PTK2 | AGGCAGTATTGACAGGGAGG | 2386 | TCATCTTGTTGATGAGCTCACC | 2387 |
| PTK2 | CGACAGCAACAGGAAATGG | 2388 | GGAATGGTCTCATCCACAGTG | 2389 |
| PTK2 | TGAGACTCTCTCGAGGCAGTATT | 2390 | TTGATGAGCTCACCCAGGTC | 2391 |
| PTK2 | CGTCTAATCCGACAGCAACA | 2392 | TCATCCACAGTGGCCAATAA | 2393 |
| PTK2 | ATCAGCGCTGGCTGGAAAAAGAG | 2394 | ATCCACAGTGGCCAATAATGTCCTCAG | 2395 |
| PTK2 | ATGGAAGAAGATCAGCGCTGGCTG | 2396 | ATGGTCTCATCCACAGTGGCCAAT | 2397 |
| PTK2 | CTAATCCGACAGCAACAGGAAAT | 2398 | AGGAGGGGAATGGTCTCATC | 2399 |
| PTK2 | ACCTGATGTGAGACTCTCTCGAG | 2400 | GCTGGTAGGAGGGGAATGGT | 2401 |
| PTK2 | GCTGACAGCTACAACGAGGGTGTCAAG | 2368 | TTTCACCAGGCCCGTCACATT | 2353 |
| PTPN13 | GACTCCTCATCCATTGAAGACC | 2404 | CCAAGCCATACTTTGCATCTTT | 2405 |
| PTPN13 | CTGTTGCGAGTTTAAATAGAAGTCC | 2406 | CCCTTTCTGGTGAAGATACTATGC | 2407 |
| PTPN13 | AGTCCTGAAAGGAGGAAACATG | 2408 | CAGGTTCACTAAGGTGATCTCCC | 2409 |
| PTPN13 | TTCAAAGTCTGTTGCGAGTTTAAA | 2410 | GTGATCTCCCTTTCTGGTGAAG | 2411 |
| PTPN13 | GGAGGAAACATGAATCAGACTCC | 2412 | TCACTAAGGTGATCTCCCTTTCTG | 2413 |
| PTPN13 | TAAATAGAAGTCCTGAAAGGAGGAAAC | 2414 | GAAGATACTATGCTCCATCTTTTGTGT | 2415 |
| PTPN13 | CCTGGGCAAGCATATGTTCTAG | 2416 | AAGCATCCATCCAAGTCAGC | 2417 |
| PTPN13 | CATGAATCAGACTCCTCATCCA | 2418 | TCCAGTCTTCCCATCTTCTCC | 2419 |
| PTPN13 | ATTGAAGACCCTGGGCAAG | 2420 | GGGCAACTGAACTGATAAATATGC | 2421 |
| PTPN13 | CATCCATTGAAGACCCTGGG | 2422 | CCATCTTCTCCCCACCAATA | 2423 |
| PTPN13 | AAGACCCTGGGCAAGCATAT | 2424 | CTGGCTTCAAGCATCCATCC | 2425 |
| PTPN13 | TGAATCAGACTCCTCATCCATTGAAGA | 2426 | ATCCATCCAAGTCAGCTGGTCC | 2427 |
| PTPN13 | ACAAACCGTTGCAGAGTTGG | 2428 | AATATGCCTAGGTCCAGTCTTCC | 2429 |
| PTPN13 | CCTTCTCACCAGATGTCAAGATC | 2430 | TGAACTGATAAATATGCCTAGGTCC | 2431 |
| PTPN13 | GATGCAGAATCTTTGGCAGG | 2432 | AAGTCAGCTGGTCCTCCAGG | 2433 |
| PTPN13 | CCTCTCTCTATCCACATCGGAA | 2434 | TCCCCACCAATAATTTCAAATC | 2435 |
| PTPN13 | AGATCTGATGCAGAATCTTTGGC | 2436 | CCTAGGTCCAGTCTTCCCATCTT | 2437 |
| PTPN13 | GCAGAGTTGGTGGGAAAACC | 2438 | TCCAGGGGCAACTGAACTGA | 2439 |
| PTPN13 | TGTCATTGTTAACATGGAACCC | 2440 | ATCTTCTCCCCACCAATAATTTGA | 2441 |
| PTPN13 | TCACCAGATGTCAAGATCTGATGC | 2442 | CTGGTCCTCCAGGGGCAACT | 2443 |
| PTPN13 | TGCAGAATCTTTGGCAGGAGTGAC | 2444 | GGCTTCAAGCATCCATCCAAGTCA | 2445 |
| PTPN13 | CAGAGTTGGTGGGAAAACCTTCTCAC | 2446 | CCTCCAGGGGCAACTGAACTGATAAAT | 2447 |
| PTPN13 | TGGCAGGAGTGACAAAACTTAA | 2448 | CACACTATTCACAGATATCAAACGG | 2449 |
| PTPN13 | GATGTCAAGATCTGATGCAGAATC | 2450 | CCCTCCAGACTCACACTATTCAC | 2451 |
| PTPN13 | GGAAAACCTTCTCACCAGATGTC | 2452 | TCCAGACTCACACTATTCACAGATATC | 2453 |
| PTPN13 | GAATCTTTGGCAGGAGTGACAAAACTT | 2454 | ATTGCAGCATGGTGGCTGAC | 2455 |
| PTPN13 | CCACCACAAACCGTTGCAGA | 2456 | TGGCTGACTCCCTCCAGACT | 2457 |
| PTPN13 | CCGTTGCAGAGTTGGTGGGA | 2458 | AGCATGGTGGCTGACTCCCT | 2459 |
| PTPN13 | GAACCCCCACCACAAACC | 2460 | CTGACTCCCTCCAGACTCACACT | 2461 |
| RAD52 | GCCTCAAGTCCAAGGCTTTAT | 2462 | CAGTTTCCAAGTGCATTCCC | 2463 |
| RAD52 | GTTGGTTATGGTGTTAGTGAGGG | 2464 | CAGTTTCCAAGTGCATTCCC | 2463 |
| RAD52 | GCCTCAAGTCCAAGGCTTTAT | 2462 | GCGTGGAAGCTTATTTAGTGATC | 2466 |
| RAD52 | TGGTTCATATCATGAAGATGTTGG | 2467 | TGATCTCAGGTAGTCTTTGTCCAG | 2468 |
| RAD52 | GTGTTAGTGAGGGCCTCAAGTC | 2469 | GTCCAGAATACAGTTTCCAAGTGC | 2470 |
| RAD52 | TCATGAAGATGTTGGTTATGGTG | 2471 | TCAGGTAGTCTTTGTCCAGAATACAG | 2472 |
| RAD52 | TGAGGGCCTCAAGTCCAAGG | 2473 | AGTCTTTGTCCAGAATACAGTTTCCAA | 2474 |
| RAD52 | GGTTATGGTGTTAGTGAGGGCCTCAAG | 2475 | AATACAGTTTCCAAGTGCATTCCCAAA | 2476 |
| RAD52 | CAAGGCTTTATCTTTGGAGAAGG | 2477 | GGCAGCTGTTGTATCTTGCC | 2478 |
| RAD52 | AGAAGGCAAGGAAGGAGGC | 2479 | TCCACAGACGGTTCAAGATCT | 2480 |
| RAD52 | GTGACAGACGGGCTGAAGC | 2481 | TGTATCTTGCCTCCTCCACAG | 2482 |
| RAD52 | CAAGTCCAAGGCTTTATCTTTGG | 2483 | AGCTGTTGTATCTTGCCTCCTCC | 2484 |
| RAD52 | AAGGAAGGAGGCGGTGACAG | 2485 | ATGTTCGGTCGGCAGCTGTT | 2486 |
| RAD52 | GCTGAAGCGAGCCCTCAG | 2487 | CGGTATCACAGCATGGCTG | 2488 |
| RAD52 | AGACGGGCTGAAGCGAGCC | 2489 | TGGGTCTGGAAGGGGAGGTC | 2490 |
| RAD52 | AGGCGGTGACAGACGGGCT | 2491 | AGCATGGCTGGGTCTGGAAG | 2492 |
| RAD52 | ATTTGTGAGGGTCCAGCTGA | 2493 | GTTAAATCCACTTCAAGAGGCAA | 2494 |
| RAD52 | GAGTCTGTGCATTTGTGAGGG | 2495 | CTTGTCTCTTCGCTTTAGTTAAATCC | 2496 |
| RAD52 | ACGTGGGAGTCTGTGCATTT | 2497 | TCGCTTTAGTTAAATCCACTTCAAG | 2498 |
| RAD52 | AGTTCTACGTGGGAGTCTGTGC | 2499 | GGTTCAAGATCTTGTCTCTTCGC | 2500 |
| RAD52 | CTGTGCATTTGTGAGGGTCCAGC | 2501 | TGCCTCCTCCACAGACGGTT | 2502 |
| RAD52 | AATGGCAAGTTCTACGTGGG | 2503 | TCAAGATCTTGTCTCTTCGCTTTAGTT | 2504 |
| RAD52 | GATCAGTGGGTGGTAGGAGAAG | 2505 | AGCTGTGGGTGTCCCAGG | 2506 |
| RAD52 | CGGAAGGATCAGTGGGTGGTA | 2507 | AGGGCCATGTTCGGTCGG | 2508 |
| RAD52 | ATGGAGTAGACCTGCTGCCC | 2509 | GTGTCCCAGGGCCATGTTC | 2510 |
| RAD52 | CTGCCCGGAAGGATCAGT | 2511 | GCTGCAGCTGTGGGTGTC | 2512 |
| RAD52 | CAAGTCCAAGGCTTTATCTTTGG | 2483 | TGATCTCAGGTAGTCTTTGTCCAG | 2468 |
| RAD52 | CAAGGCTTTATCTTTGGAGAAGG | 2477 | TCAGGTAGTCTTTGTCCAGAATACAG | 2472 |
| RAD52 | GTGACAGACGGGCTGAAGC | 2481 | GTTAAATCCACTTCAAGAGGCAA | 2494 |
| RAD52 | GCTGAAGCGAGCCCTCAG | 2487 | TCGCTTTAGTTAAATCCACTTCAAG | 2498 |
| RAD52 | AGGAGGCGGTGACAGACG | 2521 | TGTCTCTTCGCTTTAGTTAAATCCA | 2522 |
| RAD52 | GACGGGCTGAAGCGAGCC | 2523 | ACGGTTCAAGATCTTGTCTCTTCG | 2524 |
| SHMT1 | GAACACTGCCATGTGGTGAC | 2525 | CATAGCTTGCTTCAGTGCCA | 2526 |
| SHMT1 | AGATTGCAGATGAGAACGGG | 2527 | CATAGCTTGCTTCAGTGCCA | 2526 |
| SHMT1 | GAACACTGCCATGTGGTGAC | 2525 | TATTTTGTAGCCCAGCTCCG | 2530 |
| SHMT1 | ACTCCCGAAACCTGGAATATG | 2531 | CTTCAGTGCCACAGCAACC | 2532 |
| SHMT1 | TGACCACCACCACTCACAAG | 2533 | TCAGACAGAGCCCTGCAGTT | 2534 |
| SHMT1 | GTATCTCATGGCGGACATGG | 2535 | TTTAAATTCCAGAGTCATAGCTTGC | 2536 |
| SHMT1 | GCTACGGAAGATTGCAGATGAG | 2537 | CCTGGTGTTGATAAACTTTAAATTCC | 2538 |
| SHMT1 | CCCATTTGAACACTGCCATG | 2539 | TGTGACTATTTTGTAGCCCAGC | 2540 |
| SHMT1 | ATGAGAACGGGGCGTATCTC | 2541 | CCAGAGTCATAGCTTGCTTCAGT | 2542 |
| SHMT1 | TGGCATGATCTTCTACAGGAAAG | 2543 | GTAGCCCAGCTCCGTCAGG | 2544 |
| SHMT1 | GACATGGCTCACATCAGCG | 2545 | GTCAGGGCCTCAGACAGAGC | 2546 |
| SHMT1 | CACCACTCACAAGACCCTGC | 2547 | CAGTTGGCCACCACCTGGT | 2548 |
| SHMT1 | AATATGCCCGGCTACGGAAG | 2549 | CCACCACCTGGTGTTGATAAAC | 2550 |
| SHMT1 | TGCCCTCCCCATTTGAACAC | 2551 | GACTATTTTGTAGCCCAGCTCCGTCAG | 2552 |
| SHMT1 | CTGCTACTCCCGAAACCTGG | 2553 | AGCCCTGCAGTTGGCCAC | 2554 |
| SHMT1 | AACGGGGCGTATCTCATGGC | 2555 | AGCTTGCTTCAGTGCCACAGCAA | 2556 |
| SHMT1 | TGCCGAGCTGGCATGATCTT | 2557 | GCTCCGTCAGGGCCTCAGAC | 2558 |
| SHMT1 | ATGGCGGACATGGCTCACAT | 2559 | GAGTCATAGCTTGCTTCAGTGCCACAG | 2560 |
| SHMT1 | TTGCAGATGAGAACGGGGCGTAT | 2561 | AGTTGGCCACCACCTGGTGTTGAT | 2562 |
| SHMT1 | CAAGACCCTGCGAGGCTG | 2563 | GGATCAAATGGTTGTCAGAACC | 2564 |
| SHMT1 | CCGAGCTGGCATGATCTTCTACAG | 2565 | CCATCTGTGCCTTTGGAACG | 2566 |
| SHMT1 | GCCATGTGGTGACCACCA | 2567 | ACAGGCTTCTAGCACCTTCTCA | 2568 |
| SHMT1 | CACTCACAAGACCCTGCGAGGCT | 2569 | TCTGTGCCTTTGGAACGGAGATC | 2570 |
| SHMT1 | ACATCAGCGGGCTGGTGG | 2571 | TGGAACGGAGATCCACAAGG | 2572 |
| SHMT1 | GAGGCTGCCGAGCTGGCAT | 2573 | GAACGGAGATCCACAAGGATCAAA | 2574 |
| SHMT1 | CGTGGTGCCCTCCCCATTT | 2575 | GAGATCCACAAGGATCAAATGGTTGT | 2576 |
| SHMT1 | GCTGGCGTGGTGCCCTCC | 2577 | GGCTTCTAGCACCTTCTCAGCCCTTC | 2578 |
| SHMT1 | GGCTCACATCAGCGGGCTG | 2579 | CCTTCTCAGCCCTTCCACCAT | 2580 |
| SHMT1 | AAACCTGGAATATGCCCGG | 2581 | CTTCCACCATCTGTGCCTTT | 2582 |
| SHMT1 | GCCCGGCTACGGAAGATTGC | 2583 | TCAGCCCTTCCACCATCTGTGC | 2584 |
| SLIT2 | GGCAAGTTTCAACCATATGCC | 2585 | GGAGCCATAAATGACTGGTGAC | 2586 |
| SLIT2 | AAACAACCTGTATTGTGACTGCC | 2587 | GGAGCCATAAATGACTGGTGAC | 2586 |
| SLIT2 | CCTCGGGTTGGTCTGTACAC | 2588 | TGGTCTCTGGAAGATTTGTGG | 2589 |
| SLIT2 | TCGACTGCATTCAAACAACC | 2590 | TGAGACCTTTCCCACGACAG | 2591 |
| SLIT2 | CCACCTGAGAGGCCATAATG | 2592 | CCCACGACAGTCTACGATATTG | 2593 |
| SLIT2 | GCCATAATGTAGCCGAGGTTC | 2594 | TTTCTGTGATGGTCTCTGGAAG | 2595 |
| SLIT2 | GACTGGCTTCGCCAAAGG | 2596 | CGATATTGTTGCTACAGGTACAGG | 2597 |
| SLIT2 | AAACGAGAATTTGTCTGCAGTG | 2598 | AAGATTTGTGGGGATCTCAGTG | 2599 |
| SLIT2 | GGGTTGGTCTGTACACTCAGTG | 2600 | TGCAAAACACTACAAGAAGGAGC | 2601 |
| SLIT2 | CTGCATTCAAACAACCTGTATTG | 2602 | CAAGAAGGAGCCATAAATGACTG | 2603 |
| SLIT2 | TGTATTGTGACTGCCACCTGG | 2604 | GGGATCTCAGTGAGACCTTTCC | 2605 |
| SLIT2 | TGAGAGGCCATAATGTAGCCG | 2606 | ACCTTTCCCACGACAGTCTACG | 2607 |
| SLIT2 | TGTACACTCAGTGTATGGGCCC | 2608 | CTACAGGTACAGGCGGCAGG | 2609 |
| SLIT2 | AGCCGAGGTTCAAAAACGAG | 2610 | CTCTGGAAGATTTGTGGGGATCT | 2611 |
| SLIT2 | GGCTCTCCGACTGGCTTC | 2612 | ACAGTCTACGATATTGTTGCTACAGG | 2613 |
| SLIT2 | AAAGGCCTCGGGTTGGTCT | 2614 | AGGGCAGTGCAAAACACTACA | 2615 |
| SLIT2 | TCTCCGACTGGCTTCGCCAA | 2616 | TCTGTGATGGTCTCTGGAAGATTTGTG | 2617 |
| SLIT2 | GCTTCGCCAAAGGCCTCG | 2618 | GTACAGGCGGCAGGGCAGTG | 2619 |
| SLIT2 | CCCTCCCACCTGAGAGGC | 2620 | TCTCAGTGAGACCTTTCCCACGAC | 2621 |
| SLIT2 | CCGAGGTTCAAAAACGAGAATTTG | 2622 | TTTGTGGGGATCTCAGTGAGACCT | 2623 |
| SLIT2 | CACCTGGCCTGGCTCTCC | 2624 | AACACTACAAGAAGGAGCCATAAATG | 2625 |
| SLIT2 | TAATGTAGCCGAGGTTCAAAAAC | 2626 | TGATTGTGTTCTGTTCCAAACG | 2627 |
| SLIT2 | GGCCTGGCTCTCCGACTG | 2628 | AGGGATGACTTTGATTGTGTTCTG | 2629 |
| SLIT2 | AGTGTATGGGCCCCTCCCAC | 2630 | ATGACTTTGATTGTGTTCTGTTCCAAA | 2631 |
| SLIT2 | GTTGGTCTGTACACTCAGTGTATGGGC | 2632 | TGAGAAAGCTCCAGGAGGGAT | 2633 |
| SLIT2 | GGTTCAAAAACGAGAATTTGTCTGCAG | 2634 | GAAAGCTCCAGGAGGGATGACTTT | 2635 |
| SLIT2 | CTCCCACCTGAGAGGCCATAATGTAGC | 2636 | CTCCAGGAGGGATGACTTTGATTGTG | 2637 |
| SLIT2 | TGACTGCCACCTGGCCTGG | 2638 | TTGGAAAGCATCTGGTGCAAG | 2639 |
| SLIT2 | ATGGGCCCCTCCCACCTGAG | 2640 | GGAAAGCATCTGGTGCAAGTTCAGAG | 2641 |
| SLIT2 | GGCAAGTTTCAACCATATGCC | 2585 | TTATATGGTGAGAAAGCTCCAGG | 2643 |
| SLIT2 | TCAACCATATGCCTAAACTTAGGAC | 2644 | TCTAAGGTTTTTATATGGTGAGAAAGC | 2645 |
| SLIT2 | TTTCTGTGGCAAGTTTCAACC | 2646 | GAGATCTGATTATTGCTCAGGTCA | 2647 |
| SLIT2 | ACTAGACTTTCTGTGGCAAGTTTCA | 2648 | TCTGGTGCAAGTTCAGAGATCTGA | 2649 |
| SLIT2 | CAACATTACTAGACTTTCTGTGGCA | 2650 | GTAGTCCTTGGAAAGCATCTGG | 2651 |
| STIM1 | ATCGAGATCCTCTGTGGCTTC | 2652 | GAACACTGCTCTGCAGGCTAG | 2653 |
| STIM1 | TGTGGCTTCCAGATTGTCAAC | 2654 | ATGCTGTGGCTCCGTCAG | 2655 |
| STIM1 | CAACCCTGCTCACTTCATCA | 2656 | CTCTGAGATCCCAGGCCAT | 2657 |
| STIM1 | AGATTGTCAACAACCCTGGC | 2658 | GCTGCCGAACACTGCTCT | 2659 |
| STIM1 | ATCCACTCACTGGTGGCTGC | 2660 | AGATCCCAGGCCATGCTGTG | 2661 |
| STIM1 | AAGCACTGAGCGAGGTGACA | 2662 | AGGCCATGCTGTGGCTCC | 2663 |
| STIM1 | GCTTCCAGATTGTCAACAACCCT | 2664 | CAGGCGCTGCCGAACACT | 2665 |
| STIM1 | ACCGCTGGCAACAGATCGAG | 2666 | GTGGCTCCGTCAGGCGCT | 2667 |
| STIM1 | GACGTGGATGACATGGATGA | 2668 | AATCGGAATGGGTCAAATCC | 2669 |
| STIM1 | TTGTGTCTCCCTTGTCCATG | 2670 | AGCGCCAGTAATGCTTCTT | 2671 |
| STIM1 | ACATGGATGAGGAGATTGTGTCT | 2672 | AAGTCATGGCATTGAGAGCC | 2673 |
| STIM1 | TTCATCATGACTGACGACGTG | 2674 | GCAGTTTCTCCACCAGAGACC | 2675 |
| STIM1 | AGCTGGATGGGCAGTACACG | 2676 | GTCACTCATGTGGAGGGAGG | 2677 |
| STIM1 | GAGGAGATTGTGTCTCCCTTGT | 2678 | AGTAATGCCTTCTTGGCCAG | 2679 |
| STIM1 | CCTCAACATAGACCCCAGCT | 2680 | ATTGAGAGCCTCGTCTGCAG | 2681 |
| STIM1 | GCTCACTTCATCATGACTGACG | 2682 | GAGGACTCCGAATCGGAATG | 2683 |
| STIM1 | ATGACTGACGACGTGGATGAC | 2684 | GCTCATCTGAGGAGGTTTGG | 2685 |
| STIM1 | TGGATGACATGGATGAGGAGAT | 2686 | CTGCCATTGGAAGTCATGG | 2687 |
| STIM1 | AACCCTGGCATCCACTCACT | 2688 | GCTGGCGGTCACTCATGT | 2689 |
| STIM1 | AGTACACGCCCCAACCCT | 2690 | CATTGGAAGTCATGGCATTG | 2691 |
| STIM1 | GCTGCCCTCAACATAGACCC | 2692 | AGCACGGCTCATCTGAGGAG | 2693 |
| STIM1 | ACATAGACCCCAGCTGGATG | 2694 | ATGGCATTGAGAGCCTCGTC | 2695 |
| STIM1 | TCAACAACCCTGGCATCCAC | 2696 | CTCCGAATCGGAATGGGTCA | 2697 |
| STIM1 | ATCCTCTGTGGCTTCCAGATT | 2698 | GGAGGGAGGACTCCGAATC | 2699 |
| STIM1 | ACTGACGACGTGGATGACATGGAT | 2700 | CACCAGAGACCCTGGGTGGAC | 2701 |
| STIM1 | ACTGGTGGCTGCCCTCAACATA | 2702 | GTCTGCAGCACGGCTCATCT | 2703 |
| STIM1 | GAGATTGTGTCTCCCTTGTCCATGCAG | 2704 | CTCGATCAGCCGGTGGCTG | 2705 |
| STIM1 | GCAACAGATCGAGATCCTCTGTGG | 2706 | CACACGCTGGCGGTCACTC | 2707 |
| STIM1 | CAACCCTGCTCACTTCATCATGACTGA | 2708 | CGGTGGCTGCCATTGGAAGT | 2709 |
| STIM1 | ATGGGCAGTACACGCCCCAA | 2710 | GAGCCTCGTCTGCAGCACGG | 2711 |
| STIM1 | ACGCCCCAACCCTGCTCACT | 2712 | ATCAGCCGGTGGCTGCCATT | 2713 |
| STIM1 | ACCCCAGCTGGATGGGCAGT | 2714 | AGGAGGTTTGGGGGCCACAC | 2715 |
| SYK | CACAACTTCCAGGTTCCCAT | 2716 | CCCAGTTCTTTGTCTTCCAGC | 2717 |
| SYK | CACAACTTCCAGGTTCCCAT | 2716 | CTTTGTCTGCAGCCCAGG | 2718 |
| SYK | GAAATGTTAATTTTGGAGGCCG | 2719 | CCAGGGTGCAAGTTCTGG | 2720 |
| SYK | AATTTTGGAGGCCGTCCACAAC | 2721 | CTGCAGCCCAGGGTGCAAGT | 2722 |
| SYK | CCGTCCACAACTTCCAGGTT | 2723 | TTCTGGCTCATACGGATTGAA | 2724 |
| SYK | CTTCCAGGTTCCCATCCTGC | 2725 | AGGGTGCAAGTTCTGGCTCATA | 2726 |
| SYK | CGAGCATTATTCTTATAAAGCAGATG | 2727 | ATGACACAGTACTCTCTTGCCG | 2728 |
| SYK | GAGTTCTTACTGTCCCATGTCAAA | 2729 | GCTCATACGGATTGAATGACAC | 2730 |
| SYK | GGTTTGTTAAGAGTTCTTACTGTCCC | 2731 | TACTCTCTTGCCGGTTCCCTT | 2732 |
| SYK | GACAACAACGGCTCCTACGC | 2733 | TGCAAGTTCTGGCTCATACGGATT | 2734 |
| SYK | TGCACGAAGGGAAGGTGCTG | 2735 | GACACAGTACTCTCTTGCCGGTTCCCT | 2736 |
| SYK | CCAGAGACAACAACGGCTCC | 2737 | TTCCCTTGGGCAGGGGAG | 2738 |
| SYK | TCCTACGCCCTGTGCCTGCT | 2739 | GCCGGTTCCCTTGGGCAG | 2740 |
| SYK | AGCAGATGGTTTGTTAAGAGTTCTTAC | 2741 | CCCAGTTCTTTGTCTTCCAGC | 2717 |
| SYK | CGAGGGAAAGAAGTTCGACA | 2743 | TCGTACACCTCTGTGTCCATG | 2744 |
| SYK | CACTATCGATCGACAAAGACA | 2745 | ATGGGTAGGGCTTCTCTCTGG | 2746 |
| SYK | CAAAGACAAGACAGGGAAGCTC | 2747 | GTAGGGGCTCTCGTACACCTC | 2748 |
| SYK | AGAAGTTCGACACGCTCTGG | 2749 | AGGTAAACCTCCTTGGGCCT | 2750 |
| SYK | AAGACAGGGAAGCTCTCCATC | 2751 | CTCTGTGTCCATGGGTAGGG | 2752 |
| SYK | GCATCGACAAAGACAAGACAGG | 2753 | TGTCCATGGGTAGGGCTTCT | 2754 |
| SYK | AGGGAAAGAAGTTCGACACGCTCT | 2755 | TCCTTGGGCCTGATCTCCTC | 2756 |
| SYK | GAAGCTCTCCATCCCCGAG | 2757 | GGGCTCTCGTACACCTCTGTGTC | 2758 |
| SYK | ATCCCCGAGGGAAAGAAGTT | 2759 | TCGGTCCAGGTAAACCTCCT | 2760 |
| SYK | CTGTGCCTGCTGCACGAAGG | 2761 | CTGTGTCCATGGGTAGGGCTTCTCTCT | 2762 |
| SYK | TCTCCATCCCCGAGGGAAAG | 2763 | TAAACCTCCTTGGGCCTGATCTC | 2764 |
| SYK | TGCTGCACTATCGCATCGAC | 2765 | GGTCCGCGTAGGGGCTCTC | 2766 |
| SYK | AAGGGAAGGTGCTGCACTATC | 2767 | CTGATCTCCTCGGGGTCC | 2768 |
| SYK | CCTGCTGCACGAAGGGAAGG | 2769 | TCCTCGGGGTCCGCGTAGG | 2770 |
| SYNE2 | CTCACGAAGAGGACGAGGAG | 2771 | TTGCTTGTAGTGATGCTCGG | 2772 |
| SYNE2 | GAACCGTCATCTCCTCAGTCC | 2773 | TCCTGTCACCTTCATTTGC | 2774 |
| SYNE2 | TCCTCAGTCCCTGTGTCATCTA | 2775 | CACCTTCCATTTGCTTGTAGTG | 2776 |
| SYNE2 | GGCCTCTGAGAATGAAACAGAC | 2777 | GCCATCCGAAATGGATTTAC | 2778 |
| SYNE2 | CTGATTCTTGGCGTAAACGG | 2779 | GAACAGGTGGAACATTCCTGTC | 2780 |
| SYNE2 | GAATGAAACAGACATGGAAGACC | 2781 | GTAGTGATGCTCGGGACAGG | 2782 |
| SYNE2 | CCTGTGTCATCTAGTGGCCC | 2783 | TGGTTTATAAGGGGTGCTGG | 2784 |
| SYNE2 | AAGACCCCAGAGAAATCCAGAC | 2785 | GAACATTCCTGTCACCTTCCA | 2786 |
| SYNE2 | GCTTGGAAGATGAAAAGGAGG | 2787 | AAGGGCTGTCGGGAACAT | 2788 |
| SYNE2 | GAAATCCAGACTGATTCTTGGC | 2789 | TTCCATTTGCTTGTAGTGATGCTC | 2790 |
| SYNE2 | AGAGAGCGAGGAACCGTCAT | 2791 | ATAGGGTGGTTTATAAGGGGTGC | 2792 |
| SYNE2 | GTCATCTCCTCAGTCCCTGTGT | 2793 | GGGAACATGCCACGAGTG | 2794 |
| SYNE2 | GTAAACGGGGAGAGAGCGAG | 2795 | CTGTCGGGAACATGCCAC | 2796 |
| SYNE2 | TGTCAGCGTGGACTCCATC | 2797 | CGGGACAGGAAGGGCTGT | 2798 |
| SYNE2 | CCCAGAGAAATCCAGACTGATTC | 2799 | GAGTGGCCATCCGAAATG | 2800 |
| SYNE2 | GACCACACAGGCGACGTG | 2801 | ATGCTCGGGACAGGAAGGG | 2802 |
| SYNE2 | GGCCCATACTACAGCGCACT | 2803 | AGGGGGAACAGGTGGAACAT | 2804 |
| SYNE2 | GGGCTCCTCCTCTCACGAAG | 2805 | ATTCCTGTCACCTTCCATTTGCTTGTA | 2806 |
| SYNE2 | CTTGGCGTAAACGGGGAGAG | 2807 | ACAGGAAGGGCTGTCGGGAA | 2808 |
| SYNE2 | AGGACGAGGAGGGCCCATACTA | 2809 | TATAAGGGGTGCTGGACGCAG | 2810 |
| SYNE2 | GCGAGGAACCGTCATCTCCT | 2811 | CTGGACGCAGGGGGAACAG | 2812 |
| SYNE2 | ATCCAGACTGATTCTTGGCGTAAACG | 2813 | ATTTGCTTGTAGTGATGCTCGGGACAG | 2814 |
| SYNE2 | CTCCTCTCACGAAGAGGACG | 2815 | CGTGCCTGGAGGTAATAGTAGC | 2816 |
| SYNE2 | CTGCGAGACCCCTGTCAG | 2817 | CTTCTTTGCCACCATCCGT | 2818 |
| SYNE2 | CTGGAGTGGGACCACACAG | 2819 | GTGGGTTGCCATTCAGGACT | 2820 |
| SYNE2 | AGACCCCTGTCAGCGTGGAC | 2821 | GTCTTCCTGCTGTGGGTTGC | 2822 |
| SYNE2 | ACTCCATCCCCCTGGAGTG | 2823 | CTGCTGCTCTGTGATACCGG | 2824 |
| SYNE2 | CACGAGCGGTCTGGCTGC | 2825 | ACCATCCGTGCCTGGAGGTAAT | 2826 |
| SYNE2 | AGTGGGACCACACAGGCGAC | 2827 | CGGGCCTTCTTTGCCACCAT | 2828 |
| SYNE2 | GAGGAGGGCCCATACTACAGCGC | 2829 | TTTGCCACCATCCGTGCCTG | 2830 |
| SYNE2 | AGCGTGGACTCCATCCCCCT | 2831 | ATTCAGGACTCGCGGGCCTT | 2832 |
| SYNE2 | GGTCTGGCTGCGAGACCC | 2833 | CTGCTGTGGGTTGCCATTC | 2834 |
| SYNE2 | ATCCCCCTGGAGTGGGACC | 2835 | GCTCTGTGATACCGGCCAGT | 2836 |
| SYNE2 | CAGGGCACGAGCGGTCTG | 2837 | TTGCCATTCAGGACTCGCGG | 2838 |
| SYNE2 | GTCATCTAGTGGCCCCAGGG | 2839 | ACTCGCGGGCCTTCTTTG | 2840 |
| SYNE2 | TAGTGGCCCCAGGGCACGAG | 2841 | GCCAGTCCCCCGTCTTCCTG | 2842 |
| SYNE2 | CGGGGAGAGAGCGAGGAACC | 2843 | TCCCCCGTCTTCCTGCTGTG | 2844 |
| TOPBP1 | TGCCCAATTCTTCAACTCCT | 2845 | TGTAGGCTCCAGTTTGCTGTT | 2846 |
| TOPBP1 | GTATGAGTGTGCCAAGAGATGG | 2847 | AATCTTCAGGTGCTTGAAATGC | 2848 |
| TOPBP1 | TTCAACTCCTACCAGCCAGATC | 2849 | CTTGAAATGCACTGACATCCAG | 2850 |
| TOPBP1 | CAAGACAGAACCTAGACCAGAAGC | 2851 | TTGATTCACTTACGCAACTTGC | 2852 |
| TOPBP1 | CCAGCCAGATCAACACAATTG | 2853 | CCGACAACCATCTAATAAATCTTCAG | 2854 |
| TOPBP1 | TGCCAAGAGATGGAATGTACAC | 2855 | CCAGTTTGCTGTTAAGTGAATTACA | 2856 |
| TOPBP1 | GACCAGAAGCAAAGACTATGCC | 2857 | CAGGTGCTTGAAATGCACTGAC | 2858 |
| TOPBP1 | TGGAATGTACACTGTGTGACCAC | 2859 | CGCAACTTGCATTTATGTTGG | 2860 |
| TOPBP1 | CCAATTCTTCAACTCCTACCAGC | 2861 | TGCACTGACATCCAGATTTTCTAG | 2862 |
| TOPBP1 | AGACTATGCCCAATTCTTCAACTC | 2863 | TTTCAAGTGTAGGCTCCAGTTTG | 2864 |
| TOPBP1 | GTCAGAAGTATGAGTGTGCCAAG | 2865 | TTGCATTTATGTTGGAAATATTGC | 2866 |
| TOPBP1 | TGTCAGGATGAATCCATATACAAGAC | 2867 | CCATCTAATAAATCTTCAGGTGCTTG | 2868 |
| TOPBP1 | TGAGAAAGGTTTTTGTCAGGATG | 2869 | TTCTAGATTTTCAAGTGTAGGCTCC | 2870 |
| TOPBP1 | CAAGAGATGGAATGTACACTGTGTGA | 2871 | CACTTACGCAACTTGCATTTATG | 2872 |
| TOPBP1 | AGAACCTAGACCAGAAGCAAAGAC | 2873 | TGCTGTTAAGTGAATTACATATTGATT | 2874 |
| TOPBP1 | ATGAGTGTGCCAAGAGATGGAATGTAC | 2875 | GTAGGCTCCAGTTTGCTGTTAAGTGAA | 2876 |
| TOPBP1 | GTGTGACCACACAGTGGTTTT | 2877 | TTATGTTGGAAATATTGCTGACAT | 2878 |
| TOPBP1 | CTCCTACCAGCCAGATCAACACA | 2879 | AAACGAACTCCACCTCCACTG | 2880 |
| TOPBP1 | TTTGACAGTATTGAGAAAGGTTTTTG | 2881 | CATGAGTTACATCTTCATTTAGCTGG | 2882 |
| TOPBP1 | GGTTTTTGTCAGGATGAATCCA | 2883 | CCACCTCCACTGTTAATAAGTCTTC | 2884 |
| TOPBP1 | ACACCTCATTGTGCAAGAACC | 2885 | CTGCCACTAAAACCGCAAAG | 2886 |
| TOPBP1 | TGAATGTACACACCTCATTGTGC | 2887 | GCTTTCTGCCACTAAAACCG | 2888 |
| TOPBP1 | AGCATGGAGGTCAATACATGG | 2889 | TCCACTGTTAATAAGTCTTCTCAGTTT | 2890 |
| TOPBP1 | CATGGAGGTCAATACATGGGACAATT | 2891 | GCTTTCTGCCACTAAAACCGCAAAGAT | 2892 |
| TOPBP1 | TCAATACATGGGACAATTGAAAAT | 2893 | CGAACTCCACCTCCACTGTTAATA | 2894 |
| TSSC4 | TTGGCTGTCCAATCACACTC | 2895 | ATGCCTCTCAGATGGAATGG | 2896 |
| TSSC4 | ATGGCTGAGGCAGGAACAG | 2897 | CTCCGTTGTCACTCATGCTG | 2898 |
| TSSC4 | GACGCATGGCTGAGGCAG | 2899 | ACAGAGGATGGAGCCCGTCT | 2900 |
| TSSC4 | CTGGGGACGCATGGCTGAG | 2901 | GGCCTGAGGGCGCTAGGG | 2902 |
| TSSC4 | CAATCACACTCCAGTGTCAACC | 2903 | CTCCAGGCAGTCAAAGATGTC | 2904 |
| TSSC4 | ATCAGGGCTCCGTCCACTTG | 2905 | GTCAAAGATGTCACGGCTGC | 2906 |
| TSSC4 | CGCTGAGGACCTTCATCAGG | 2907 | AGCTCATGCCTCTCAGATGG | 2908 |
| TSSC4 | CAGTGTCAACCACTGGCACC | 2909 | TGGGAGAAGGTGGAGCTCAT | 2910 |
| TSSC4 | AGGCCTGAGACGACCACG | 2911 | TCAGATGGAATGGCTGCAC | 2912 |
| TSSC4 | GCTGTCCAATCACACTCCAGTGT | 2913 | AAGGTGGAGCTCATGCCTCT | 2914 |
| TSSC4 | AGGACCTTCATCAGGGCTCC | 2915 | CACCGTGGCTGGGAGGAG | 2916 |
| TSSC4 | CACCCAGCAGCCAAGAGAG | 2917 | GGGCCACAGAGGATGGAG | 2918 |
| TSSC4 | CACTTGGCCCGCTTGGCTGT | 2919 | ATGGAATGGCTGCACCGTGG | 2920 |
| TSSC4 | AACCACTGGCACCCAGCAGC | 2921 | CAGGCAGTCAAAGATGTCACGGCT | 2922 |
| TSSC4 | CTGTGCCGCTGAGGACCTTC | 2923 | TGCGCTGGGAGAAGGTGGAG | 2924 |
| TSSC4 | GCCCGCTTGGCTGTCCAATC | 2925 | AGAAGGTGGAGCTCATGCCTCTCAGAT | 2926 |
| TSSC4 | ACCTTCATCAGGGCTCCGTCCAC | 2927 | AGATGTCACGGCTGCGCTGG | 2928 |
| TSSC4 | ACACTCCAGTGTCAACCACTGGC | 2929 | ACGGCTGCGCTGGGAGAAG | 2930 |
| TSSC4 | CCGTCCACTTGGCCCGCTT | 2931 | GCCCCCTCCAGGCAGTCAAA | 2932 |
| TSSC4 | CACGCCTGTGCCGCTGAG | 2933 | ATGGAGCCCGTCTGGCCG | 2934 |
| TSSC4 | AGACGACCACGCCTGTGC | 2935 | TGGTGTGGGCCACAGAGGAT | 2936 |
| TSSC4 | ACTCCGAGGCCTGAGACGAC | 2937 | TCATGCTGGTGTGGGCCAC | 2938 |
| TUBA1 | GACTCAACGTGAGACGCACC | 2939 | CTCTTTCCCAGTGATGAGCTG | 2940 |
| TUBA1 | GACTCAACGTGAGACGCACC | 2939 | CCTTGCCAATGGTATAGTGACC | 2941 |
| TUBA1 | ACTGCAGCTAGCGCAGTTCT | 2942 | CTCTTTCCCAGTGATGAGCTG | 2940 |
| TUBA1 | ACCTGTCACCCCGACTCAAC | 2943 | GGTCAATGATCTCCTTGCCA | 2944 |
| TUBA1 | CTAGCGCAGTTCTCACTGAGAC | 2945 | GTTGTTGGCAGCATCCTCTT | 2946 |
| TUBA1 | TCTCACTGAGACCTGTCACCC | 2947 | TGACCACGGGCATAGTTGTT | 2948 |
| TUBA1 | ACCCCGACTCAACGTGAGAC | 2949 | GGCATAGTTGTTGGCAGCAT | 2950 |
| TUBA1 | GTGCGGCACTGCAGCTAG | 2951 | CAGCATCCTCTTTCCCAGTG | 2952 |
| TUBA1 | ATAAGGGCGGTGCGGCACT | 2953 | TGGGTGGAAGAGCTGTCGGTAT | 2954 |
| TUBA1 | ACCGCCCGGACTCACCATG | 2955 | CCGATCCAGCACTGGGTCAAT | 2956 |
| TUBA1 | ACTGAGACCTGTCACCCCGACTC | 2957 | CAATGGTATAGTGACCACGGGC | 2958 |
| TUBA1 | AGACGCACCGCCCGGACT | 2959 | TCCAGCACTGGGTCAATGATCTC | 2960 |
| UTRN | CAAACACCCTCGACTTGGTT | 2961 | TGGCAATACTGCTGGATGAG | 2962 |
| UTRN | CAAACACCCTCGACTTGGTT | 2961 | TGTGGCATATTGTTCTATTCTTGAA | 2963 |
| UTRN | TGGGGAAGATGTACGAGACTTC | 2964 | ACAGTTGAGGAGATTGTGAGGG | 2965 |
| UTRN | CAGGTCGAAGAAGTACTTTGCC | 2966 | TTCTTGAATGGGTGTCATCATG | 2967 |
| UTRN | CAACATCTGGGGAAGATGTACG | 2968 | TTGTTCTATTCTTGAATGGGTGTC | 2969 |
| UTRN | AAGAACAAGTTCAGGTCGAAGAAG | 2970 | ATCATGAAACAGTTGAGGAGATTG | 2971 |
| UTRN | GGTACTTAAGAACAAGTTCAGGTCG | 2972 | GAATGGGTGTCATCATGAAACAG | 2973 |
| UTRN | TACTTTGCCAAACACCCTCG | 2974 | GACCCATTAGTCCTTTCCATCTG | 2975 |
| UTRN | GACTTGGTTACCTGCCTGTCC | 2976 | ACACTTCCTGTGGTGGAGCT | 2977 |
| UTRN | TCCAGACAGTTCTTGAAGGTGAC | 2978 | CAGTGAGAAAAGACCCATTAGTCC | 2979 |
| UTRN | CGAAGAAGTACTTTGCCAAACAC | 2980 | TGGTGGAGCTGCTATCAGTG | 2981 |
| UTRN | ACCCTCGACTTGGTTACCTGC | 2982 | AGTCCTTTCCATCTGGGCCA | 2983 |
| UTRN | CGAGACTTCACAAAGGTACTTAAGAA | 2984 | GCTGCTATCAGTGAGAAAAGACC | 2985 |
| UTRN | CTTTGCCAAACACCCTCGACTTGG | 2986 | TGGTGGAGCTGCTATCAGTGAGAAAAG | 2987 |
| UTRN | AGAAGTACTTTGCCAAACACCCTCGAC | 2988 | ACTTCCTGTGGTGGAGCTGCTATCAGT | 2989 |
| UTRN | TCACAAAGGTACTTAAGAACAAGTTCA | 2990 | TGGCAATACTGCTGGATGAG | 2962 |
| UTRN | ACAAGTTCAGGTCGAAGAAGTACTTTG | 2992 | TCCGAGTGTTTGGCAATACTG | 2993 |
| UTRN | CACAAATTACATTACCCAATGGTG | 2994 | GACTGTCCTCCGAGTGTTTGG | 2995 |
| UTRN | CCCAATGGTGGAATATTGTATACC | 2996 | ATACTGCTGGATGAGGGCGT | 2997 |
| UTRN | GAGTTGTTTCTTTTCGGGTCG | 2998 | GAGGGCGTGCTCGTCTTC | 2999 |
| UTRN | GCAAAAGGTCACAAATTACATTACC | 3000 | AGTGTTTGGCAATACTGCTGGAT | 3001 |
| UTRN | TTTTCGGGTCGAACAGCAAAAG | 3002 | CTGGATGAGGGCGTGCTCGT | 3003 |
| UTRN | ACATTACCCAATGGTGGAATATTG | 3004 | ACTGGGGACTCTCCTCCGAG | 3005 |
| UTRN | TCGAACAGCAAAAGGTCACA | 3006 | CTGGCTCACTGGGGACTCTC | 3007 |
| UTRN | TCGAACAGCAAAAGGTCACAAATTACA | 3008 | ACTCTCCTCCGAGTGTTTGGCAATACT | 3009 |
| UTRN | TGTTTCTTTTCGGGTCGAACAGC | 3010 | TCTGCGGCTGGCTCACTG | 3011 |
| UTRN | CTGCCAGAGTTGTTTCTTTTCG | 3012 | CAGGATCTGAGCTGGGCTCT | 3013 |
| UTRN | TGATGTCTGCCAGAGTTGTTTC | 3014 | TGACTTCAGGATCTGAGCTGG | 3015 |

Generally, one forward and one reverse primer are designed for each predicted exon-exon junction, as shown in FIG. 1B. To eliminate artefacts, the design algorithm generates at least two independent primer pairs for each AceView predicted event. In this way each alternative splicing event is validated by two independent PCR reactions. For the gene set analyzed in this study, an average of 37 primers was designed per gene. The primer sets are designed to amplify fragments ranging between 100-400 base pairs. It was found that this size range provides optimal accuracy during capillary electrophoresis separation of the amplicons and hence facilitates the automatic identification and assignment of amplicons (FIGS. 10 and 10).

### Example 2

### Data Collection and Analysis

Normal and serous epithelial ovarian cancer tissue samples were obtained as frozen specimens from the Cancer Research Network of the FRSQ. Histopathology, grade and stage were assigned according to the International Federation of Gynecology and Osbtetrics (FIGO) criteria. Only chemotherapy naïve tumor samples were used in the study. RNA Extraction from 50 mg tissue samples was done using TRIZOL® Reagent according to the manufacturer's protocol, using a PowerMax™ homogenizing system equipped with a 10 mm saw tooth blade (VWR International). To retain maximum yield of RNA, DNase treatment was not performed. Extracted RNA was isopropanol precipitated, then resuspended in pure water and stored at −80° C. RNA concentration was quantified on an Agilent 2100 BioAnalyzer (Agilent technologies). Typical total RNA yields of 1 to 66 μg per 50 mg specimen were obtained.

Ovarian tissues were classified as normal or cancerous according to the relative expression profile of the genes KRT18, KRT7, VIM, CDH1, TERT relative to GAPDH as measured by QPCR using PCR primers flanking dual fluorescent probes (see Table 3). Eight normal and eight tumour RNA samples showing expression data closest to the median for each gene's expression level for the normal or tumour tissue type were selected and combined in equal amounts to formulate 2 normal and 2 tumour pools of 4 samples each. Tissue quality control was established using real-time PCR amplification of known genes with known cancer or tissues type specific expression profile including the epithelial cell markers KRT7, KRT18 and CDH1, the stromal marker vimentin, and the tumour cell content indicator hTERT (Table 3). KRT7, KRT18 and CDH1 were shown to the upregulated in high grade serous ovarian cancer (Chu & Weiss, 2002, Mod Pathol, 15: 6-10; Ouellet et al., 2005, Oncogene, 24: 4672-4687; Sun et al., 2007, Eur J Obstet Gynecol Reprod Biol, 130: 249-257).

TABLE 3
Primer and dual labelled fluorescent probes used for quantitative PCR.

| GENE | Forward | Probe¹ | Reverse |
|---|---|---|---|
| CDH-1 | AATTCACCCAGGAGGTCTTTA | CTCCATCACAGAGGTTCCTGGAA | TTGGCTGAGGATGGTGTAA |
| | (SEQ ID N0: 3016) | GA (SEQ ID NO: 3017) | (SEQ ID NO: 3018) |
| KRT18 | TTCGCAAATACTGTGGACAA | CCAGCTCTGTCTCATACTTGACT | CCCATGGATGTCGTTCTC |
| | (SEQ ID NO: 3019) | CTAAAGTCA | (SEQ ID NO: 3021) |
| | | (SEQ ID NO: 3020) | |
| KRT7 | GCTGCTGAGAATGAGTTT | TAGGCAGCATCCACATCCTTCTT | GGTCCTGAGGAAGTTGATCTC |
| | GTG(SEQ ID NO: 3022) | CA (SEQ ID NO: 3023) | (SEQ ID NO: 3024) |
| TERT | TGTGCACCAACATCTACAAGA | CGTGAAACCTGTACGCCTGCAG | AGGCCGTGTCAGAGATGA |
| | (SEQ ID NO: 3025) | (SEQ ID N0: 3026) | (SEQ ID NO: 3027) |
| VIM | TCTTGACCTTGAACGCAAA | CCTGGATTTCCTCTTCGTGGAGT | CATGCTGTTCCTGAATCTGA |
| | (SEQ ID NO: 3028) | TT (SEQ ID NO: 3029) | (SEQ ID NO: 3030) |

¹Dual labelled fluorescent probe, with 5′-FAM, 3′-TAMRA.

Tissues that fail the quality control were considered to have low tumour tissue content or reflect different or aberrant tumour subset, and were not considered further in the study. For the quality control, tissues (normal and cancerous) are first classified based on histopathological assessment. Moreover, since the portion of tissue used for subsequent analysis may be from a different region of the tumor that been examined by pathologists, one must assess the quality of the tissue that will be used following classification by pathologists by comparing expression levels of the 5 genes with the median expression levels for all tissues of a given type (normal or tumour) as called by histopathological assessment. Normal versus tumour tissues have different expression patterns for these 5 genes.

Reverse transcription was performed on 2 μg total RNA samples in the presence of RNAse inhibitor according to the manufacturers' protocols. Reactions were primed with both (dT)21 and random hexamers at final concentrations of 1 μM and 0.9 μM respectively. The integrity of the cDNA was assessed by SYBR® Green based quantitative PCR, performed on three housekeeping genes: MRPL19, PUM1 and GAPDH using primers illustrated in Table 4.

TABLE 4
Primer used for SYBR Green based quantitative.

| | | SEQ | | SEQ |
|---|---|---|---|---|
| | | ID | | ID |
| GENE | Forward | NO: | Reverse | NO: |
| BMP4 | TCCACAGCACTGGTCTTGAG | 3031 | GATCACCTCGTTCTCAGGGA | 3032 |
| CHEK2 | GCGCCTGAAGTTCTTGTTTC | 3033 | GCCTTTGGATCCACTACCAA | 3034 |
| DNMT3B | CCATGCAACGATCTCTCAAA | 3035 | CAGCAGAAACTTTGATGGCA | 3036 |
| FN1 | ACCTGGAGGAGACCACATGA | 3037 | TACCATCATCCAGCCTTGGT | 3038 |
| HMGA1 | GCGAAGTGCCAACACCTAAG | 3039 | GAGATGCCCTCCTCTTCCTC | 3040 |
| HSC20 | ATACAGCGAAGCTCCAGCAC | 3041 | AGGGTCGAATGCTTCTCTGA | 3042 |
| UTRN | CTCATCCAGCAGTATTGCCA | 3043 | CTGGTCCTTCAGCTGCTCAT | 3044 |
| SYNE2 | TCACCCAGTCCTTACAACTCC | 3045 | CATCAACGTCACCCTTCCTC | 3046 |
| SHMT1 | CAATGACGATGCCAGTCAAC | 3047 | AACCCTCTGCCGGTTACTCT | 3048 |
| PTK2 | TCCGGAGGGTCTGATGAA | 3049 | GTGAACCAGGGTAGCCAGAA | 3050 |
| KITLG | CATTGCCAGCATTGTTTTCT | 3051 | TGTATATTTTCAACTGCCCTTGT | 3052 |
| GAPDH | GTGAAGGTCGGA | 3053 | TGCCATGGGTGG | 3054 |
| | GTCAACGGATTT | | AATCATATTGGA | |
| PUM1* | TGAGGTGTGCACCATGAAC | 3055 | CAGAATGTGCTTGCCATAGG | 3056 |
| MRPL19* | GGGATTTGCAT | 3057 | GGAAGGGCA | 3058 |
| | TCAGAGATCAG | | TCTCGTAAG | |

*sequences reported in Szabo et al. (2004, Genome Biol, 5: R59)

Ct (quantitative PCR cycle threshold) values for these genes, typically in the range of 14-25, depending on the gene, were used to verify the integrity of each cDNA sample. These Ct values are determined using standard SYBR green QPCR methods on an Eppendorf Mastercycler thermocycler. Following QPCR, the samples were analyzed by capillary electrophoresis to ensure that only one amplicon of the expected size was obtained.

PCR reactions were performed on 20 ng cDNA in 10 μl final volume containing 0.2 mM each dNTP, 1.5 mM MgCl2, 0.6 μM each primer and 0.2 units of Taq DNA polymerase. An initial incubation of 2 minutes at 95° C. was followed by 35 cycles at 94° C. 30 s, 55° C. 30 s, and 72° C. 60 s. The amplification was completed by a 2 minute incubation at 72° C.

RNA quantification and integrity analysis was performed on an Agilent bioanalyzer (Agilent, Santa Clara, Calif.), using the manufacturer's software. Analysis of the DNA amplification reactions was performed on Caliper LabChip® 90 instruments (Caliper LifeSciences, Hopkinton, Mass.), and amplicon sizing and relative quantification was performed by the manufacturer's software, prior to being uploaded to the LISA database.

The LISA was built around the LAMP solution stack of software programs (Linux operating system, Apache web server, Mysql database management server and Perl and Python programming languages). In addition, several peripheral Perl and Python modules for experimental design, analysis, and display of results interact with the LISA. Statistical t-tests and unsupervised clustering were performed using the R package.

The capillary electrophoresis instrument software (Caliper LifeSciences, Hopkinton, Mass.) provides size and concentration data for the detected peaks of each PCR reaction. These data are uploaded to the LISA database and compared with expected amplicon sizes for that experiment. Using the experimentally determined amplicon sizing data, a signal detection protocol assigns detected amplicons to expected sizes. Gene sequence, primer sequence, single nucleotide polymorphism sites and protein coding data are associated to each element of experimental data stored in the database.

For each PCR reaction covering an AS event, the concentration data from all RNA sources under consideration were used to determine the most prevalent assigned amplicon. For each RNA source, the ratio of the concentration of this amplicon to the total assigned amplicon concentrations measured is calculated and is expressed as a percentage, termed the percent splicing index, (PSI or Ψ). Ψ values for each reaction are used to compare alternative splicing profiles between RNA sources. Percent splicing index, Ψ values for different RNA sources are used in statistical t-tests, and resulting p-values are used in the screening process to determine cancer specificity. Reaction sets with Bonferroni-corrected p-values of less than 0.0002 were considered statistically significant hits.

The designed sets of PCR experiments are passed to the automated platform together with associated experimental conditions such as the RNA source, and PCR reaction conditions. Once the PCR amplification and capillary electrophoresis are completed, an electropherogram is generated that reflects the amplification pattern, as shown in FIG. 1D. The electropherogram is analyzed by the LISA and the detected amplicons are compared with the expected amplicon sizes and assigned correspondingly. If the detected peaks do not match some or all of the predicted amplicons, these peaks are labelled as “unassigned” and are stored in the database for subsequent novel splicing event analysis. In the present study, 4% of the primers failed to amplify a product. Failure of specific primers was easily offset by built-in redundancy in the PCR reaction design. On completion of the RT-PCR based gene annotation, the results obtained from different RNA sources are compared to identify sample-specific patterns of splicing. As shown in FIG. 1D, the alternative splicing event tested in Stim1, displayed a variable splicing pattern in these RNA sources. These results demonstrate that LISA can detect sample-specific differences in splicing patterns.

### Example 3

### Annotation and Display of Validated Splicing Events

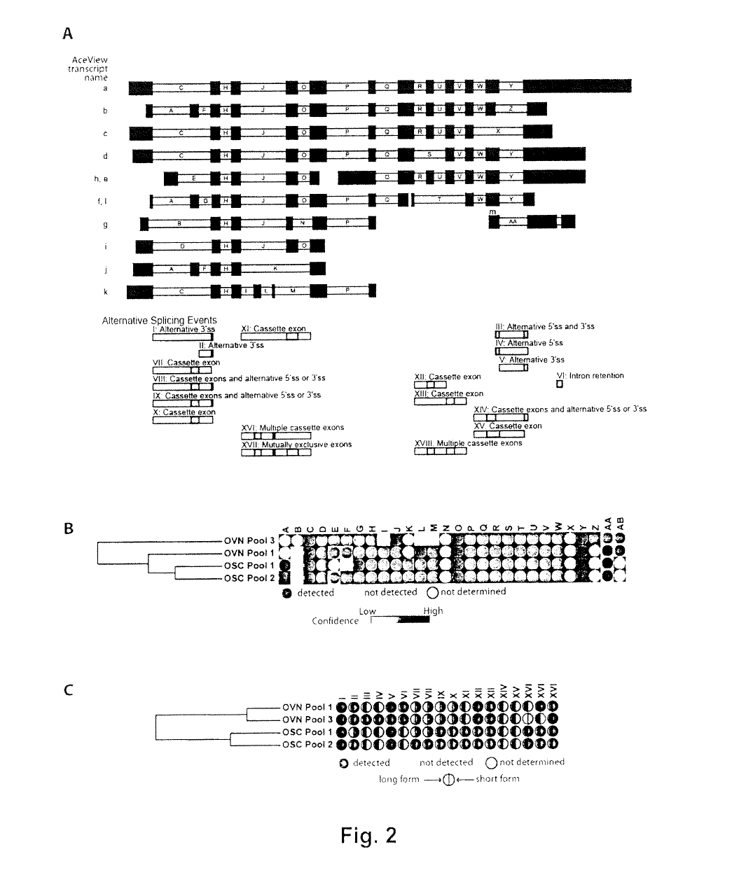

In addition to the tissue specific representation shown in FIG. 1E, two additional representations of the annotation of splicing events have been developed within the LISA. As shown for the ovarian cancer associated gene SHMT1 (FIG. 2A), each intron is uniquely labelled, while transcript names are retained from AceView. By comparing different AceView transcripts, the LISA generates all potential alternative splicing events and each event is assigned a unique number. After the RT-PCR analysis of the gene as illustrated in FIGS. 1A to 1D, the expression of exon-exon junctions are displayed as “detected”, “not detected” or “not determined” with a confidence level ranging from low to high (FIG. 2B). In the case of SHMT1, 2 predicted exon junctions out of 28 were “not detected” in pools of RNA obtained from normal and ovarian serous tissues of 16 individuals with a very high degree of confidence (all primer sets in agreement). On the other hand, 3 exon-exon junctions were found to be tissue type-dependent with medium confidence rating (FIG. 2B, columns A, K and AB). Three exon-exon junctions were not determined in one RNA source (OVN Pool 3 in I, L and M) and one junction was not detected with high level confidence in any RNA source tested (FIG. 2B, column X). To monitor the relative expression of each splicing isoform, a schematic representation of each predicted splicing isoform is also generated by LISA. In this display (FIG. 2C), the long form generated by each alternative splicing event of SHMT1 is shown in green and the short form in red. Four splicing events were RNA source-specific and one short and one long form were not detected in one RNA source. Sequencing of selected source-specific amplicons identified by LISA confirmed the detection accuracy. This demonstrates the capacity of LISA to produce exon-exon or splice events specific annotation and that SHMT1 is spliced in a tissue-specific manner.

### Example 4

### Comparative Profiling of Splicing Events in Normal and Serous Ovarian Tumour Tissues

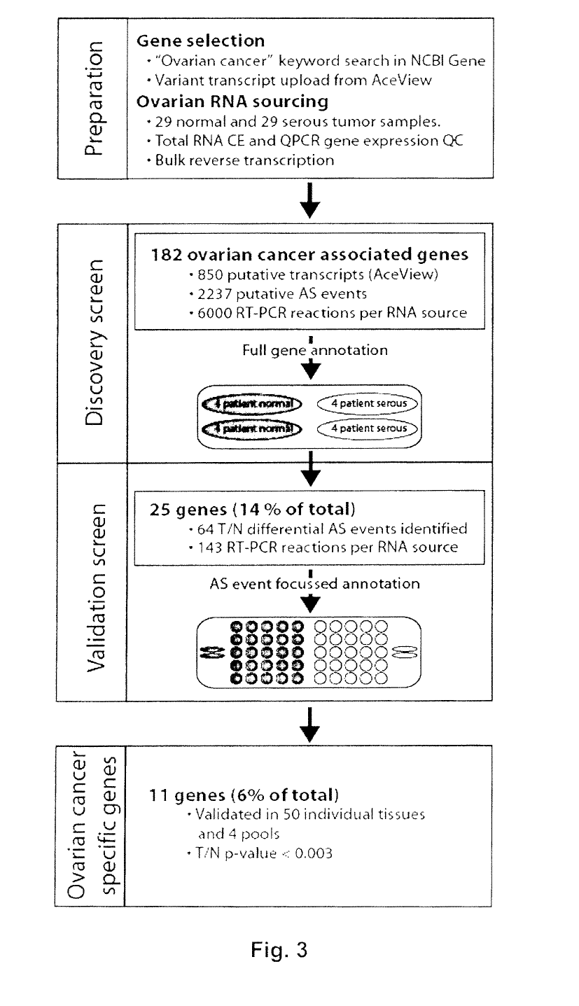

To identify cancer associated splicing events, a LISA based screening pipeline was constructed for genes associated with ovarian cancer (FIG. 3). Candidate ovarian cancer-associated genes were identified by a keyword search in public databases, yielding a list of 600 genes. All genes were entered into LISA for the identification of alternative splicing events and experimental design. A total of 4709 alternative splicing events were identified and 19 800 PCR reactions were designed. The screen was divided in two stages: the first termed the discovery screen and the second, the validation screen.

The discovery screen was carried out using two pools of RNA extracted from high-grade (grades 2 and 3; grades are standard clinical classification of tumor that take into account the size and invasive status of the tumor) serous epithelial ovarian cancer specimens and two pools of RNA extracted from unmatched normal ovaries (same age group and no prior chemotherapy). Each pool contained an equivalent mix of four independent tissues. Normal ovarian tissues were selected from women undergoing oophorectomy for reasons other than ovarian cancer, and the normality of the ovaries was confirmed by standard pathology tissue examination. Ovaries with benign tumours or cysts were excluded. Most of the donors were postmenopausal women of the age group when most serous tumours develop. Screening of the two cancers and two normal pools identified 104 cancer associated splicing events in 98 different genes. Alternative splicing events identified in the discovery screen were re-examined on individual RNA samples extracted from 25 serous epithelial ovarian cancers (grades 2 and 3) and 21 normal ovary samples. Overlapping PCR reactions using RNA from each tissue were carried out as in the discovery screen confirming the association of 48 alternative splicing events in 45 genes with ovarian cancer tissues. The other 56 events identified in the discovery phase were found to be associated only with a subclass of cancer tissues and thus may represent either differences between individuals or cancer subtypes. Table 5 gives the gene name and the percent of cancer tissue samples lying outside the range of the normal samples (defined as the percent of identification in Table 5). For example, 50% means half of the cancer tissue samples did not overlap with any of the normal tissue samples. The second column gives the p-values that characterize statistical separation between the cancer and normal populations.

TABLE 5
Gene name and the percent of cancer tissue samples lying outside the
range of the normal samples (percent of identification).

| Gene | Percent of Identification | MannWhitney_pvalue |
|---|---|---|
| BCMP11 | 60 | 5.09E−007 |
| C11orf17 | 75 | 1.55E−007 |
| CHEK2 | 0 | 3.93E−004 |
| DNMT3B | 90 | 3.25E−009 |
| BMP4 | 30 | 4.14E−005 |
| GNB3 | 55 | 2.19E−005 |
| GATA3 | 0 | 5.51E−005 |
| KITLG | 80 | 1.58E−005 |
| PAXIP1 | 85 | 2.84E−011 |
| PLD1 | 70 | 6.06E−008 |
| STIM1 | 100 | 5.51E−009 |
| NRG1 | 94.12 | 1.50E−006 |
| RAD52 | 40 | 7.72E−003 |
| SYNE2 | 95 | 1.23E−008 |
| TOPBP1 | 55 | 3.34E−008 |
| UTRN | 80 | 3.16E−007 |
| SLIT2 | 50 | 3.74E−005 |
| FN1EDB | 0 | 2.74E−006 |
| FN1EDA | 40 | 2.80E−004 |
| FN1IICSUP | 75 | 3.30E−007 |
| FN1IICSDOWN | 85 | 2.34E−008 |
| APC | 90 | 7.47E−008 |
| APP | 95 | 1.71E−009 |
| AXIN1 | 70 | 3.38E−006 |
| BTC | 40 | 4.96E−003 |
| CCNE1 | 40 | 5.25E−005 |
| FANCA | 35 | 1.11E−003 |
| FANCL | 10 | 3.98E−009 |
| FGFR1 | 40 | 1.62E−002 |
| FGFR2 | 40 | 1.05E−003 |
| FGFR4 | 0 | 1.05E−005 |
| IGSF4 | 75 | 2.24E−008 |
| LGALS9 | 30 | 5.20E−006 |
| MCL1 | 40 | 5.19E−006 |
| NUP98 | 45 | 1.60E−006 |
| POLI | 0 | 2.94E−004 |
| PTPN13 | 60 | 2.29E−007 |
| SYK | 65 | 7.63E−006 |
| TSSC4 | 45 | 3.96E−005 |
| TUBA1 | 0 | 1.72E−004 |
| C11ORF4 | 75 | 1.05E−007 |
| POLM | 30 | 2.60E−007 |
| PSAP | 75 | 2.08E−008 |
| HMGA1 | 0 | 1.80E−004 |
| PTK2 | 95 | 1.80E−008 |
| AFF3 | 55 | 2.11E−007 |
| SHMT1 | 20 | 4.50E−004 |
| HSC20 | 85 | 2.11E−007 |

This indicates that of the 600 genes associated with ovarian cancer, breast cancer and/or DNA damage/repair tested, 45 (7.5%) harboured at least one ovarian cancer-specific splicing event.

Following the discovery screen, candidate reactions covering AS events were selected for the validation screen using the Ψ values for the 4 pools. Reactions showing a difference of at least 10 percentage points between the mean Ψs for normal and tumour pools and a maximum standard deviation of the Ψs for each tissue type not exceeding 26% were selected. Following the validation screen, Ψ values were used in a t-test for significant differences between normal and tumour tissue samples. Reaction sets using Bonferroni corrected p-values<0.0002 were selected (see Table 5).

Graphical displays were generated with Perl-based analysis modules. The modules analyze the transcript map and capillary electrophoresis data obtained for each experiment data, and apply RNA source based unsupervised clustering of the results prior to generating the displays.

The entire discovery screen annotation dataset was queried to identify unassigned amplicons which were present in more than one pool sample, and which satisfy one of the following conditions: i) amplicon detected in normal pools only, ii) amplicon detected in tumour pools only, iii) amplicon detected in normal and tumour pools, but at least double the concentration one pool type relative to the other. Candidate amplicons identified by this in silico database query were purified by agarose gel electrophoresis and sequenced.

### Example 5

### Alternative Splicing of Cassette Exons is Enriched in Serous Ovarian Cancer

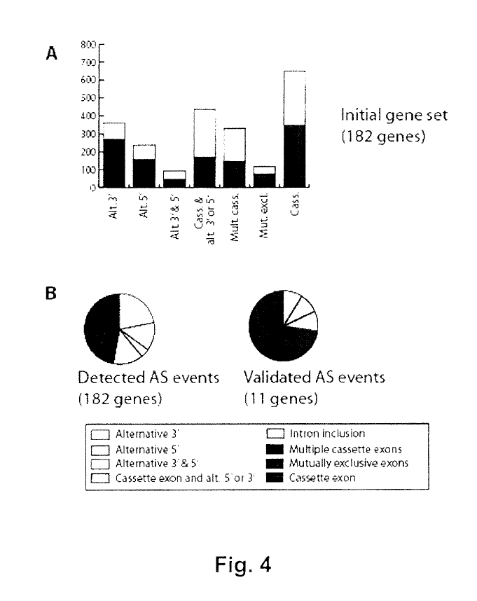

LISA-based analysis of alternative splicing automatically detects all types of alternative splicing with the exception of alternative intron inclusion, which requires special attention (FIGS. 4A and 4B). PCR amplicons generated from intron inclusion events are difficult to distinguish from those generated from genomic DNA contamination. Therefore, amplicons representing putative intron inclusion events were removed from the analysis pipeline for separate characterization. All other types of splicing events were divided into 7 groups based on the categories indicated at the bottom of FIG. 4B. Cassette exons were the most frequent alternative splicing event in the 182 gene subset considered, followed by alternative 3′, and then alternative 5′ splice events. The least frequent event was mutually exclusive alternative exons. Overall, about half of all EST-predicted alternative splicing events were validated. The general distribution of detected alternative events was slightly different than that of the predicted set. The main difference was an overrepresentation of alternative 3′ and alternative 5′ splice sites in the validated set (compare relative contribution of detected (black) and not detected (grey) events in FIG. 4A). The difference is likely due to the fact that the EST prediction is based on a large number of tissues while the data presented here are obtained only from ovarian tissues. Inspection of all validated cancer associated alternative events reveals the presence of one intron inclusion event and a large enrichment in alternative cassette exons (FIG. 4B). Indeed, 80% of the alternative splicing events associated with ovarian tumour involved alternative cassette exons. No alternative 3′ splice site events or mutually exclusive exons were part of the ovarian cancer signature. These data demonstrate that specific alterations in splicing control are occurring in serous ovarian cancers.

### Example 6

### Identification of a Novel Ovarian Cancer-Specific Alternative Splicing Event

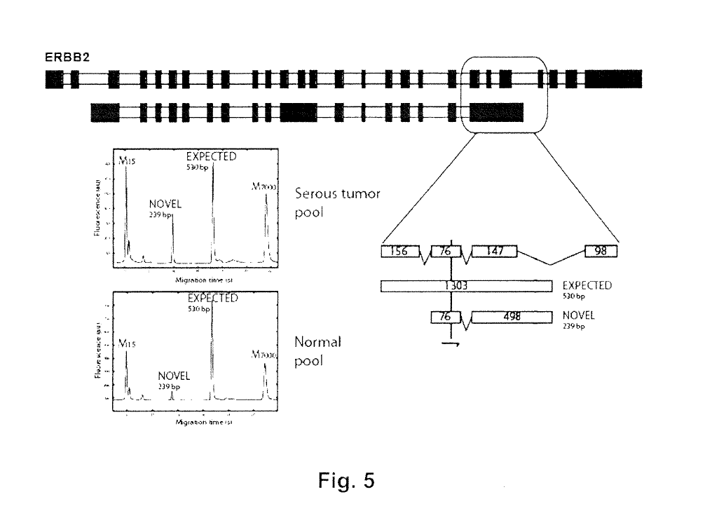

When unpredicted amplification products were obtained, they were automatically classified as “unassigned” and stored for subsequent analysis. Sequencing of eight such products appearing in RNA samples from at least two different ovarian tissues revealed that only one represented a truly novel splicing event. Others were derived from the amplification of unrelated sequence or the amplification of unspliced DNA fragments. As shown in FIG. 5, the novel cancer-specific splice junction was found in ERBB2, wherein the sequence consist of:

(SEQ ID NO: 3061)
ggttcaccca ccagagtgat ntgtggagtt atggtgtgac tgtgtgggag ctgatgactt
ttggggccaa accttacgat gggatcccag cccgggagat ccctgacctg ctggaaaagg
gggagnnnnt gccccagccc cccatatgca ccattgatgt ctacatgatc atggtcaaat
gtgcgtggct gagctgtgct ggctgcctgg aggagggtgg gaggtcct.

A new splice site was found in the middle of a previously unspliced exon leading to the generation of two alternative splicing isoforms.

### Example 7

### Use of Alternative Splicing to Diagnose Ovarian Cancer

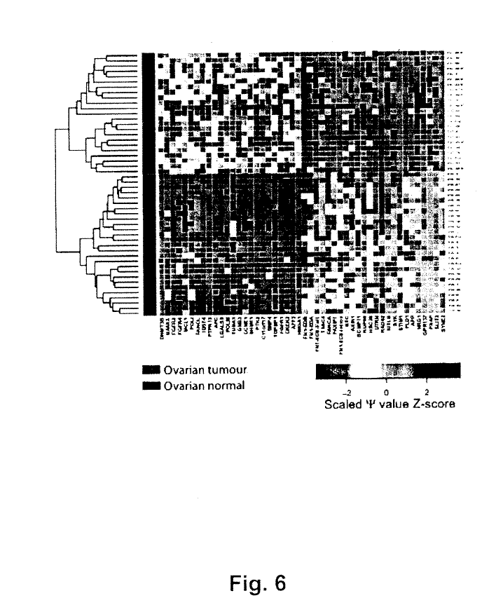

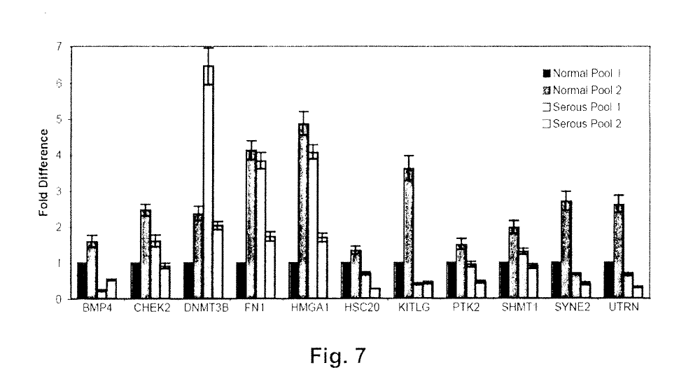

To evaluate the potential of the alternative splicing events identified by LISA as diagnostic markers for ovarian cancer, their individual and collective capacity to accurately differentiate between normal and cancer tissues was evaluated. The variance in the splicing pattern of each of the identified 48 ovarian cancer associated splicing events (Table 1) was calculated as percent splicing index (PSI, ψ) and was used to classify the 46 individual and 4 pools of normal and tumour tissues based on splicing similarity (FIG. 6). Strikingly, all tumour tissues clustered together. Visual examination of the splicing patterns present at opposing ends of the tissue cluster revealed that tissues could be classified as accurately by splicing events producing either the short or the long isoform (FIGS. 1 & 6). For example, in the case of DNMT3B, the short form is predominant in normal tissues while the long form (potential gain of a protein domain) is more abundant in cancer tissues. In contrast, SYNE2 displays a gain in protein sequence specifically in the normal tissue. The expression levels of the 45 genes listed in Table 1 were determined by quantitative PCR. As shown in FIG. 7, expression levels varied up to 5-fold between the 2 normal pools, and up to 3-fold between the 2 tumour pools. The overall expression level was consistently higher in both normal pools than in the cancer pool for 5 genes and was similar for 4 genes, while lower for 1 gene. The maximum expression level difference between normal and tumour was observed for KITLG, which showed a 9-fold higher expression in normal pool 1 compared to tumour pool 1. A correlation between shifts in alternative splicing and expression levels was not detected, and thus, for this data, expression and cancer specific-alternative splicing are distinctly regulated, as suggested previously by comparing tissue-specific expression and splicing profiles (Pan et al., 2004, Mol Cell, 16: 929-941). Consequently, the alternative splicing patterns is an efficient classifier that distinguishes serous epithelial ovarian cancer from normal ovarian tissues.

### Example 8

### Identification of Alternative Splicing Event Specific to Breast Cancer

Similarly to the methodology that is described hereinabove, the LISA based screening was used to identify a signature of diagnostic markers for breast cancer. The approach used differed in that only putative alternative splicing events, as opposed to all exon-exon junctions, were targeted by PCR primer pairs. This reduced the average number of PCR reactions per gene from 54 used for the ovarian tissue screen to 5 for the breast tissue screen of 600 genes.

TABLE 6
Properties of breast cancer-specific alternative splicing (AS) variants.

| | GENE | STRAND | ASE size, type (+ long in |
|---|---|---|---|
| GENE NAME | SYMBOL | DIRECTION | cancer, − short in cancer) |
| ADAM metallopeptidase domain 15 | ADAM15 | 1 | +75 nt exon, +72 nt exon |
| (metargidin) | | | |
| breast carcinoma amplified sequence 1 | BCAS1 | −1 | +66 nt exon |
| G protein-coupled receptor 137 | C11ORF4 | 1 | −150 nt exon |
| chemokine (C-C motif) ligand 4 | CCL4 | 1 | +113 nt exon |
| catenin (cadherin-associated protein), | CTNNA1 | 1 | −71 nt exon |
| alpha 1, 102 kDa | | | |
| discoidin domain receptor family, | DDR1 | 1 | +111 nt exon |
| member 1 | | | |
| DBF4 homolog B (S. cerevisiae). | DRF1 | 1 | +65 nt alt 3′ |
| desmocollin 3 | DSC3 | −1 | −43 nt exon |
| epithelial cell transforming sequence 2 | ECT2 | 1 | −93 nt exon |
| oncogene | | | |
| endothelial cell growth factor 1 (platelet- | ECGF1 | −1 | +274 nt intron |
| derived) | | | |
| coagulation factor III (thromboplastin, | F3 | −1 | +160 nt exon |
| tissue factor) | | | |
| fibronectin 1 | FN1 | −1 | +267 nt exon |
| cancer susceptibility candidate 4 | H63 | 1 | −168 nt exon |
| high mobility group AT-hook 1 | HMGA1 | 1 | +51 nt exon |
| hyaluronan-mediated motility receptor | HMMR | 1 | −48 nt exon |
| (RHAMM) | | | |
| insulin receptor | INSR | −1 | −36 nt exon |
| ligase III, DNA, ATP-dependent | LIG3 | 1 | −230 nt alt 3′ |
| ligase IV, DNA, ATP-dependent. | LIG4 | −1 | +73 nt exon |
| encoding Notch homolog 3 (Drosophila) | NOTCH3 | −1 | −156 nt exon |
| proprotein convertase subtilisin/kexin | PACE4 | −1 | +144 nt exon |
| type 6 | | | |
| polymerase (DNA directed), beta | POLB | 1 | −58 nt exon |
| protein tyrosine phosphatase, receptor | PTPRB | −1 | +264 nt exon |
| type, B | | | |
| CAP-GLY domain containing linker | RSN | −1 | +228 nt alt 3′, +117 nt exon |
| protein 1 | | | |
| CAP-GLY domain containing linker | RSN | −1 | −33 nt exon |
| protein 1 | | | |
| runt-related transcription factor 2 | RUNX2 | 1 | −66 nt exon |
| SHC (Src homology 2 domain containing) | SHC1 | −1 | +54 nt exon |
| transforming protein 1 | | | |
| tousled-like kinase 1 | TLK1 | −1 | −69 nt exon |
| CD40 molecule, TNF receptor | TNFRSF5 | 1 | −96 nt alt 3′ |
| superfamily member 5 | | | |

Consequently, the LISA methodology was efficient to identify specific signatures for two unrelated types of cancer (ovarian cancer and breast cancer).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for diagnosis or prognosis of a cancer in a subject by detecting a signature of splicing events comprising the steps of:
a) obtaining a nucleic acid sample from said subject, and
b) determining whether the nucleic acid sample from step a) contains a signature specific to cancer.

2. The method of claim 1, wherein said cancer is selected from the group consisting of breast, glioma, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma, testicular cancer, oral cancer, pharyngeal cancer, pediatric neoplasms, leukemia, neuroblastoma, retinoblastoma, glioma, rhabdomyoblastoma and sarcoma.

3. The method of claim 1, wherein said signature comprises at least 1 splicing variant.

4. The method of any one of claim 1, further comprising an initial step of designing at least two independent PCR primer pairs for each predicted exon-exon junction from a transcript map of a gene affected in cancer.

5. The method of claim 4, further comprising a step of PCR amplifying the nucleic acid sample with the PCR primer pairs to obtain amplicons.

6. The method of claim 4, further comprising the step of measuring the size and sequence of said amplicons.

7. The method of any one of claim 1, wherein said splicing variants occur in genes selected from the group consisting of AFF3, AGR3, APP, AXIN1, BMP4, BTC, C11orf17, CADM1, CCNE1, CHEK2, DNMT3B, FANCA, FANCL, FGFR1, FGFR2, FGFR4, FN1-EDA, FIN-EDB, FIN-IIICS, GATA3, GNB3, GPR137, HMGA1, HSC, KITLG, LGALS9, MCL1, NRG1, NUP98, PAXIP1, PLD1, POLI, POLM, PSAP, PTK2, PTPN13, RAD52, SHMT1, SLIT2, SRP19, STIM1, SYK, SYNE2 TOPBP1, TSSC4, TUBA4A UTRN, ADAM15, BCAS1, C11ORF4, CCL4, CTNNA1, DDR1, DRF1, DSC3, ECGF1, ECT2, FN1, F3, H63, HMGA1, HMMR, INSR, LIG3, LIG4, NOTCH3, PACE4, POLB, PTPRB, RSN, RUNX2, SHC1, TLK1 and TNFRSF5.

8. (canceled)

9. The method of any one of claim 3, wherein said splicing events are selected from the group consisting of an alternative 3′ splicing, an alternative 5′ splicing, an alternative 3′ and 5′ splicing, a cassette exon and alternative 5′ or 3′ splicing, a multiple cassette exons splicing, a mutually exclusive exons splicing, a cassette exon splicing, and alternative cassette exons splicing.

10. (canceled)

11. The method of claim 3, wherein said at least one splicing variant is SEQ ID NO:3061.

12. A method for identifying a signature specific of a cancer, said signature consisting of at least one specific splicing event or a specific combination of splicing events, said method comprising the steps of:
a) designing at least two independent PCR primer pairs for each predicted exon-exon junction from a transcript map of a gene affected in cancer;
b) reverse transcribing a template from RNA from a sample of cancer tissue and a sample from normal tissue;
c) amplifying amplicons of said gene by PCR with the PCR primer pairs using the template reverse transcribed from the cancer tissue and the normal tissue;
d) determining the size and sequence of said amplicons;
e) performing a comparative analysis of amplicons obtained from the template reverse transcribed from the cancer tissue and the normal tissue; and
f) identifying the presence of at least one alternative splicing event in the gene;
wherein the presence of said at least one alternative splicing event corresponds to the signature of the cancer.

13.-15. (canceled)

16. The method of any one of claim 12, wherein said PCR primer pairs are designed to amplify amplicons ranging from 100 to 700 base pairs.

17. (canceled)

18. The method of any one of claim 12, wherein said splicing event is selected from the group consisting of an alternative 3′ splicing, an alternative 5′ splicing, an alternative 3′ and 5′ splicing, a cassette exon and alternative 5′ or 3′ splicing, a multiple cassette exons splicing, a mutually exclusive exons splicing and a cassette exon splicing.

19. The method of claim 18, further comprising a step g) of selecting amplicons with a difference of at least 10% of points between a mean Ψs for normal and cancer tissue and with a maximum standard deviation of the Ψs for each tissue type of at most 26%.

20. The method of any one of claim 12, wherein said gene is selected from the group consisting of AFF3, AGR3, APP, AXIN1, BMP4, BTC, C11orf17, CADM1, CCNE1, CHEK2, DNMT3B, FANCA, FANCL, FGFR1, FGFR2, FGFR4, FN1-EDA, FIN-EDB, FIN-IIICS, GATA3, GNB3, GPR137, HMGA1, HSC, KITLG, LGALS9, MCL1, NRG1, NUP98, PAXIP1, PLD1, POLI, POLM, PSAP, PTK2, PTPN13, RAD52, SHMT1, SLIT2, SRP19, STIM1, SYK, SYNE2 TOPBP1, TSSC4, TUBA4A, UTRN, ADAM15, BCAS1, C11ORF4, CCL4, CTNNA1, DDR1, DRF1, DSC3, ECGF1, ECT2, FN1, F3, H63, HMGA1, HMMR, INSR, LIG3, LIG4, NOTCH3, PACE4, POLB, PTPRB, RSN, RUNX2, SHC1, TLK1 and TNFRSF5.

21. (canceled)

22. The method of any one of claim 12, wherein said splicing event involves alternative cassette exons.

23. A diagnostic kit for detecting a signature of a cancer in a patient comprising:
a) PCR primer pairs for predicted exon-exon junctions of at least one splicing variant; and
b) a set of instructions for using said primers to generate and detect a signature specific of ovarian cancer, said signature consisting of the at least one splicing variant or a specific combination of splicing variants.

24.-25. (canceled)

26. The kit of any of claim 23, wherein said at least one splicing variant occurs in a gene selected from the group consisting of AFF3, AGR3, APP, AXIN1, BMP4, BTC, C11orf17, CADM1, CCNE1, CHEK2, DNMT3B, FANCA, FANCL, FGFR1, FGFR2, FGFR4, FN1-EDA, FIN-EDB, FIN-IIICS, GATA3, GNB3, GPR137, HMGA1, HSC, KITLG, LGALS9, MCL1, NRG1, NUP98, PAXIP1, PLD1, POLI, POLM, PSAP, PTK2, PTPN13, RAD52, SHMT1, SLIT2, SRP19, STIM1, SYK, SYNE2 TOPBP1, TSSC4, TUBA4A, UTRN, ADAM15, BCAS1, C11ORF4, CCL4, CTNNA1, DDR1, DRF1, DSC3, ECGF1, ECT2, FN1, F3, H63, HMGA1, HMMR, INSR, LIG3, LIG4, NOTCH3, PACE4, POLB, PTPRB, RSN, RUNX2, SHC1, TLK1 and TNFRSF5.

27. (canceled)

28. A method for profiling cancer in a subject by detecting a signature of splicing events comprising the steps of:
a) obtaining a nucleic acid sample from said subject, and
b) determining whether the nucleic acid sample from step a) contains a signature specific to a cancer.

29. (canceled)

30. The method of claim 28, wherein said signature comprises at least 1 splicing variant.

31. The method of any one of claim 28, further comprising an initial step of designing at least two independent PCR primer pairs for each predicted exon-exon junction from a transcript map of a gene affected in ovarian cancer.

32. The method of any one of claim 28, further comprising a step of PCR amplifying the nucleic acid sample with the PCR primer pairs to obtain amplicons.

33. The method of any one of claim 28, further comprising the step of measuring the size and sequence of said amplicons.

34. The method of any one of claim 28, wherein said splicing variants occurs in genes selected from the group consisting of AFF3, AGR3, APP, AXIN1, BMP4, BTC, C11orf17, CADM1, CCNE1, CHEK2, DNMT3B, FANCA, FANCL, FGFR1, FGFR2, FGFR4, FN1-EDA, FIN-EDB, FIN-IIICS, GATA3, GNB3, GPR137, HMGA1, HSC, KITLG, LGALS9, MCL1, NRG1, NUP98, PAXIP1, PLD1, POLI, POLM, PSAP, PTK2, PTPN13, RAD52, SHMT1, SLIT2, SRP19, STIM1, SYK, SYNE2 TOPBP1, TSSC4, TUBA4A, UTRN, ADAM15, BCAS1, C11ORF4, CCL4, CTNNA1, DDR1, DRF1, DSC3, ECGF1, ECT2, FN1, F3, H63, HMGA1, HMMR, INSR, LIG3, LIG4, NOTCH3, PACE4, POLB, PTPRB, RSN, RUNX2, SHC1, TLK1 and TNFRSF5.

35. (canceled)

36. The method of any one of claim 28, wherein said splicing events are selected from the group consisting of an alternative 3′ splicing, an alternative 5′ splicing, an alternative 3′ and 5′ splicing, a cassette exon and alternative 5′ or 3′ splicing, a multiple cassette exons splicing, a mutually exclusive exons splicing and a cassette exon splicing.

37. (canceled)

38. The method of claim 30, wherein said at least one splicing variant is SEQ ID NO:3061.
